(19) United States
(12) Patent Application Publication
(10) Pub. No.: US 2022/0033525 A1
(43) Pub. Date: Feb. 3, 2022

(54) GENERATION OF ANTIBODY-DERIVED POLYPEPTIDES BY POLYPEPTIDE CHAIN EXCHANGE

(71) Applicant: Hoffmann-La Roche Inc., Little Falls, NJ (US)

(72) Inventors: Ulrich Brinkmann, Weilheim (DE); Can Martin Buldun, Penzberg (DE); Stefan Dengle, Munich (DE); Steffen Dickopf, Penzberg (DE); Guy Georges, Habach (DE); Eike Hoffmann, Herrsching am Ammersee (DE); Sabine Imhof-Jung, Planegg (DE); David Zilian, Grunwald (DE)

(73) Assignee: Hoffmann-La Roche Inc., Little Falls, NJ (US)

(21) Appl. No.: 17/506,993

(22) Filed: Oct. 21, 2021

Related U.S. Application Data
(63) Continuation of application No. PCT/EP2020/061412, filed on Apr. 24, 2020.

(30) Foreign Application Priority Data
Apr. 25, 2019 (EP) 19171068.0

(51) Int. Cl. C07K 16/44; C07K 16/28; G01N 33/68

(52) U.S. Cl. CPC C07K 16/44; C07K 16/2896; C07K 2317/31; G01N 33/6854; C07K 2317/526; C07K 16/2809

(57) ABSTRACT

The present invention relates to a set of heterodimeric polypeptides and its uses, e.g. for generating multispecific antigen binders by polypeptide chain exchange.

### CROSS REFERENCE TO RELATED APPLICATIONS

This application is a continuation of International Application No. PCT/EP2020/061412, filed Apr. 24, 2020, claiming priority to European Application No. 19171068.0 filed Apr. 25, 2010, which are incorporated herein by reference in their entirety.

### SEQUENCE LISTING

This application contains a Sequence Listing which has been submitted electronically in ASCII format and is hereby incorporated by reference in its entirety. Said ASCII copy, created on Oct. 4, 2021, is named Sequence_Listing.txt and is 89,449 bytes in size.

### FIELD OF THE INVENTION

The present invention relates to a set of heterodimeric polypeptides and its uses, e.g. for generating multispecific antigen binders by polypeptide chain exchange.

### BACKGROUND OF THE INVENTION

Provision of the optimal combination of antigen binders in a multispecific antibody from a variety of possible antigen binders is a challenge when each resulting multispecific antibody needs to be individually provided by recombinant methods. Alternative methods are desired for screening of a plurality multispecific antibodies.

Labrijn, A. F., et al., disclosed efficient generation of stable bispecific IgG1 by controlled Fab-arm exchange (Proc. Natl. Acad. Sci. USA 110 (2013) 5145-5150). In brief, two monospecific precursor molecules of IgG-like domain arrangement with point mutations in the CH3 domains are contacted to undergo a polypeptide chain exchange to form a bispecific product molecule, which is also of IgG-like domain arrangement. Consequently, precursor molecules and product molecule are of the same size, making isolation of product molecules from unreacted precursor molecules and analysis the product molecule challenging.

Non-published prior art PCT/EP2018/078675 and PCT/EP2018/079523 disclose methods for generating multispecific antigen binders from two different precursor molecules by polypeptide chain exchange. Both precursor molecules are heterodimeric polypeptides of asymmetric domain arrangement. Both precursor molecules comprise CH3 domains modified according to the “knob-into-holes” technology (WO 96/027011, Ridgway, J. B., et al., Protein Eng. 9 (1996) 617-621; and Merchant, A. M., et al., Nat. Biotechnol. 16 (1998) 677-681) and comprising further destabilizing mutations that are arranged in an asymmetric pattern. In each one of the precursor molecules, only one of the CH3 domains comprises such destabilizing mutation. Upon polypeptide chain exchange, two product molecules are formed, wherein each one of the product molecules comprises a polypeptide from each one of the precursor molecules. Precursor molecule and product molecules are of a different domain arrangement. PCT/EP2018/078675 and PCT/EP2018/079523 disclose amino acid positions in the CH3/CH3 interface of the precursor molecules to be substituted.

Yet, there is still a need for further methods for generating multispecific antigen binders by polypeptide chain exchange.

### SUMMARY OF THE INVENTION

The present invention relates to a set of heterodimeric precursor polypeptides comprising:
a first heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation,
wherein the first heterodimeric precursor polypeptide comprises a first antigen binding moiety, wherein at least a part of the first antigen binding moiety is arranged on one of the two polypeptide chains comprising the CH3 domain, and
a second heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation,
wherein the second heterodimeric precursor polypeptide comprises a second antigen binding moiety, wherein at least a part of the second antigen binding moiety is arranged on one of the two polypeptide chains comprising the CH3 domain;

wherein
A) either i) within the first heterodimeric precursor polypeptide the polypeptide chain comprising the CH3 domain comprising the knob mutation comprises at least a part of the first antigen binding moiety and within the second heterodimeric precursor polypeptide the polypeptide chain comprising the CH3 domain with the hole mutation comprises at least a part of the second antigen binding moiety, or ii) within the first heterodimeric precursor polypeptide the polypeptide chain comprising the CH3 domain comprising the hole mutation comprises at least a part of the first antigen binding moiety and within the second heterodimeric precursor polypeptide the polypeptide chain comprising the CH3 domain with the knob mutation comprises at least a part of the second antigen binding moiety; and wherein
B) either
i) the CH3 domain of the first heterodimeric precursor polypeptide comprising the knob mutation and the CH3 domain of the second heterodimeric precursor polypeptide comprising the hole mutation, or
ii) the CH3 domain of the first heterodimeric precursor polypeptide comprising the hole mutation and the CH3 domain of the second heterodimeric precursor polypeptide comprising the knob mutation comprise the following amino acid substitutions, wherein the numbering is according to the Kabat numbering system:
the CH3 domain with the hole mutation comprises at least one amino acid substitution selected from the group of:
   replacement of S354 with a hydrophobic amino acid;
   replacement of D356 with a positively charged amino acid;
   replacement of E357 with a positively charged amino acid or with a hydrophobic amino acid;
   replacement of D356 with a positively charged amino acid, and replacement of E357 with a positively charged amino acid or with a hydrophobic amino acid;
   replacement of S364 with a hydrophobic amino acid;
   replacement of A368 with a hydrophobic amino acid;
   replacement of E392 with a negatively charged amino acid;
   replacement of T394 with a hydrophobic amino acid;
   replacement of D399 with a hydrophobic amino acid and replacement of S400 with a positively charged amino acid;
   replacement of D399 with a hydrophobic amino acid and replacement of F405 with a positively charged amino acid;
   replacement of V407 with a hydrophobic amino acid; and
   replacement of K409 with a negatively charged amino acid; and
   replacement of K439 with a negatively charged amino acid;
the CH3 domain with the knob mutation comprises at least one amino acid substitution selected from the group of.
   replacement of Q347 with a positively charged amino acid, and replacement of K360 with a negatively charged amino acid;
   replacement of Y349 with a negatively charged amino acid;
   replacement of L351 with a hydrophobic amino acid, and replacement of E357 with a hydrophobic amino acid;
   replacement of S364 with a hydrophobic amino acid;
   replacement of W366 with a hydrophobic amino acid, and replacement of K409 with a negatively charged amino acid;
   replacement of L368 with a hydrophobic amino acid;
   replacement of K370 with a negatively charged amino acid;
   replacement of K370 with a negatively charged amino acid, and replacement of K439 with a negatively charged amino acid;
   replacement of K392 with a negatively charged amino acid;
   replacement of T394 with a hydrophobic amino acid;
   replacement of V397 with a hydrophobic amino acid;
   replacement of D399 with a positively charged amino acid, and replacement of K409 with a negatively charged amino acid;
   replacement of S400 with a positively charged amino acid;
   F405W;
   Y407W; and
   replacement of K439 with a negatively charged amino acid.

One embodiment of the invention relates to the set of heterodimeric polypeptides of the invention, wherein either i) the CH3 domain comprising the knob mutation of the first heterodimeric precursor polypeptide comprises a cysteine mutation and the CH3 domain comprising the hole mutation of the second heterodimeric precursor polypeptide comprises a cysteine mutation, or ii) the CH3 domain comprising the hole mutation of the first heterodimeric precursor polypeptide comprises a cysteine mutation and the CH3 domain comprising the knob mutation of the second heterodimeric precursor polypeptide comprises a cysteine mutation.

One embodiment of the invention relates to the set of heterodimeric polypeptides of the invention, wherein the first antigen binding moiety and/or the second antigen binding moiety is an antibody fragment.

Another aspect of the invention is a method for generating a heterodimeric polypeptide comprising the steps of.
a) contacting a first heterodimeric precursor polypeptide and a second heterodimeric precursor polypeptide according to the invention to form a third heterodimeric polypeptide comprising at least one polypeptide from the first heterodimeric precursor polypeptide and at least one polypeptide from the second heterodimeric polypeptide, and
b) recovering the third heterodimeric polypeptide.

One embodiment of the invention relates to the method of generating a heterodimeric polypeptide of the invention, wherein the second heterodimeric precursor polypeptide comprises an antigen binding moiety specifically binding to a second antigen, and wherein the third heterodimeric polypeptide comprises the antigen binding moiety specifically binding to the first antigen and the antigen binding moiety specifically binding to the second antigen

Another aspect of the invention is heterodimeric polypeptide obtained by a method according to the invention.

Another aspect of the invention is a method for identifying a multispecific heterodimeric polypeptide comprising the steps of
a) generating a plurality of multispecific heterodimeric polypeptides by subjecting each combination of
   a first heterodimeric precursor polypeptide from a plurality of first heterodimeric precursor polypeptides comprising an antigen binding moiety specifically binding to a first antigen, and
   a second heterodimeric precursor polypeptide from a plurality of second heterodimeric precursor polypeptides comprising an antigen binding moiety specifically binding to a second antigen,
   to the method of generating a heterodimeric polypeptide of the invention; and
b) individually detecting a desired characteristic of each multispecific heterodimeric polypeptide from the plurality of multispecific heterodimeric polypeptide generated in step a), and
c) selecting a multispecific heterodimeric polypeptide.

Another aspect of the invention is the set of heterodimeric precursor polypeptides according to the invention for use as a medicament.

With the invention disclosed herein precursor polypeptides are provided that are capable of undergoing a polypeptide chain exchange in order to form product polypeptides. Thereby, multispecific antigen binding polypeptides may be generated. The generation of multispecific antigen binding polypeptides may occur due to combination of two precursor polypeptides comprising antigen binding moieties specifically binding to different antigens, or by activating an antigen binding site due to the polypeptide chain exchange resulting in association of antibody variable domains specifically binding to an antigen, or by both of aforementioned.

The invention provides methods for generating product polypeptides of different size than the precursor polypeptides, thus allowing easy separation of the desired product polypeptide from the unreacted precursor polypeptides. Methods and sets of polypeptides according to the invention are suitable for providing product polypeptides, e.g. bispecific or multispecific antibodies, by means of an in vitro polypeptide chain exchange and by avoiding recombinant expression of the product polypeptide. By this, methods and sets of polypeptides of the invention are suitable for screening of product polypeptides formed from a plurality of precursor polypeptides, e.g. allowing identification of advantageous combinations of antigen binding moieties. Methods and sets of polypeptides of the invention may be advantageously used for providing antigen binding polypeptides for therapeutic or diagnostic use.

Therapeutic application of the sets of precursor polypeptides of the invention allows generation of the desired product polypeptide at the target site, thus reducing off-target effects of the product polypeptide.

### DESCRIPTION OF THE FIGURES

FIG. 1: Exemplary structures of heterodimeric precursor polypeptides and corresponding product polypeptides formed upon polypeptide chain exchange. A first heterodimeric precursor polypeptide comprises three polypeptide chains: 1. A first heavy chain polypeptide comprising from N- to C-terminal direction antibody domains VH, CH1, hinge, CH2 and CH3. The CH3 domain comprises a knob mutation and a cysteine mutation, and does not comprise a destabilizing mutation. 2. A second heavy chain polypeptide comprising from N- to C-terminal direction antibody domains hinge, CH2 and CH3. A tagging moiety is fused to the C-terminus of the CH3 domain. The CH3 domain comprises a hole mutation and a destabilizing mutation. 3. A light chain polypeptide comprising from N- to C-terminal direction antibody domains VL and CL. The VH domain and the VL domain form an antigen binding site specifically binding to a first antigen. The heavy chain polypeptides of the first heterodimeric precursor polypeptide are associated with each other via their CH3 domains. The hinge region comprises interchain disulfide bonds. A second heterodimeric precursor polypeptide comprises three polypeptide chains: 1. A first heavy chain polypeptide comprising from N- to C-terminal direction antibody domains VH, CH1, hinge, CH2 and CH3. The CH3 domain comprises a hole mutation and a cysteine mutation, and does not comprise a destabilizing mutation. 2. A second heavy chain polypeptide comprising from N- to C-terminal direction antibody domains hinge, CH2 and CH3. A tagging moiety is fused to the C-terminus of the CH3 domain. The CH3 domain comprises a knob mutation and a destabilizing mutation. 3. A light chain polypeptide comprising from N- to C-terminal direction antibody domains VL and CL. The VH domain and the VL domain form an antigen binding site specifically binding to a second antigen. The heavy chain polypeptides of the second heterodimeric precursor polypeptide are associated with each other via their CH3 domains. The hinge region comprises interchain disulfide bonds. In presence of a reducing agent the interchain disulfide bonds in the hinge region are reduced, thereby destabilizing the heterodimers formed by the first and second heavy chain polypeptides and supporting a polypeptide chain exchange. As a result, heterodimeric product polypeptides are formed. A first product polypeptide comprises the two antigen binding sites, i.e. the antigen binding site from the first heterodimeric precursor polypeptide and the antigen binding site from the second heterodimeric precursor polypeptide. The first product polypeptide comprises the first heavy chain polypeptides from the first and second heterodimeric precursor polypeptides, which are associated via their CH3 domains. Both heavy chain polypeptides comprised in the first product polypeptide comprise CH3 domains that do not comprise destabilizing mutations. Both CH3 domains comprise cysteine mutations, which interact and support the formation of the heterodimeric product polypeptide. The second product polypeptide, which in this example does not comprise an antigen binding site, comprises the second heavy chain polypeptide from the first heterodimeric precursor polypeptide and the second heavy chain polypeptide from the second heterodimeric precursor polypeptide. Both heavy chain polypeptides are associated via their CH3 domains. Both CH3 domains comprise destabilizing mutations, which interact and support the formation of the heterodimeric product polypeptide. This product polypeptide comprises tagging moieties that allow purification via tag-specific chromatography.

FIG. 2: Exemplary structures of heterodimeric precursor polypeptides and corresponding product polypeptides formed upon polypeptide chain exchange, wherein polypeptide chain exchange results in the activation of an antigen binding site. A first heterodimeric precursor polypeptide comprises three polypeptide chains: 1. A first heavy chain polypeptide comprising from N- to C-terminal direction antibody domains VH, CH1, peptide connector, a VH domain derived from a first antibody and CH3. The CH3 domain comprises a knob mutation and does not comprise a destabilizing mutation. 2. A second heavy chain polypeptide comprising from N- to C-terminal direction the following antibody domains: VL domain derived from a second antibody and CH3. The CH3 domain comprises a hole mutation and a destabilizing mutation. 3. A light chain polypeptide comprising from N- to C-terminal direction antibody domains VL and CL. The N-terminal VH domain from the first heavy chain polypeptide and the VL domain from the light chain polypeptide form an antigen binding site specifically binding to a target antigen. The heavy chain polypeptides of the first heterodimeric precursor polypeptide are associated with each other via their CH3 domains. No interchain disulfide bond is formed between the first and second heavy chain polypeptide. A pair of a VH domain and a VL domain is formed between the VH domain derived from the first antibody and the VL domain from the second heavy chain polypeptide. Both variable domains are associated with each other, but do not form an antigen binding site specifically binding to an antigen. A second heterodimeric precursor polypeptide comprises three polypeptide chains: 1. A first heavy chain polypeptide comprising from N- to C-terminal direction antibody domains VH, CH1, peptide connector, a VL domain derived from the first antibody and CH3. The CH3 domain comprises a hole mutation and does not comprise a destabilizing mutation. 2. A second heavy chain polypeptide comprising from N- to C-terminal direction the following antibody domains: a VH domain derived from a third antibody and CH3. The CH3 domain comprises a knob mutation and a destabilizing mutation. 3. A light chain polypeptide comprising from N- to C-terminal direction antibody domains VL and CL. The N-terminal VH domain of the first heavy chain polypeptide and the VL domain from the light chin polypeptide form an antigen binding site specifically binding to a target antigen. The heavy chain polypeptides of the second heterodimeric precursor polypeptide are associated with each other via their CH3 domains. No interchain disulfide bond is formed between the first and second heavy chain polypeptide. A pair of a VH domain and a VL domain is formed between the VL domain derived from the first antibody and the VH domain from the second heavy chain polypeptide. Both variable domains are associated with each other, but do not form an antigen binding site specifically binding to an antigen. Upon polypeptide chain exchange, heterodimeric product polypeptides are formed. A first product polypeptide comprises the two antigen binding sites from the precursor polypeptides, i.e. the antigen binding site from the first heterodimeric precursor polypeptide and the antigen binding site from the second heterodimeric precursor polypeptide. The first product polypeptide comprises the first heavy chain polypeptides from the first and second heterodimeric precursor polypeptides, which are associated via their CH3 domains. Both heavy chain polypeptides comprised in the first product polypeptide comprise CH3 domains that do not comprise destabilizing mutations. By association of the first heavy chain polypeptides from the first and second heterodimeric precursor polypeptides a pair of a VH domain derived from a first antibody and a VL domain derived from a first antibody are formed, which form an antigen binding site specifically binding to a first antigen. This antigen binding site does not exist in any precursor polypeptide and is only formed (activated) upon polypeptide chain exchange. The second product polypeptide comprises the second heavy chain polypeptide from the first heterodimeric precursor polypeptide and the second heavy chain polypeptide from the second heterodimeric precursor polypeptide. Both heavy chain polypeptides are associated via their CH3 domains. Both CH3 domains comprise destabilizing mutations, which interact and support the formation of the heterodimeric product polypeptide. By association of the second heavy chain polypeptides from the first and second heterodimeric precursor polypeptides a new pair of a VH domain and a VL domain is formed. Both variable domains are associated in the second product polypeptide.

FIG. 3: Exemplary structures of heterodimeric precursor polypeptides and corresponding product polypeptides formed upon polypeptide chain exchange, wherein polypeptide chain exchange results in the activation of an antigen binding site. A first heterodimeric precursor polypeptide comprises three polypeptide chains: 1. A first heavy chain polypeptide comprising from N- to C-terminal direction antibody domains VH, CH1, peptide connector, a VH domain derived from a first antibody, CH2 and CH3. The CH3 domain comprises a knob mutation and does not comprise a destabilizing mutation. 2. A second heavy chain polypeptide comprising from N- to C-terminal direction the following antibody domains: a VL domain derived from a second antibody, CH2 and CH3. The CH3 domain comprises a hole mutation and a destabilizing mutation. 3. A light chain polypeptide comprising from N- to C-terminal direction antibody domains VL and CL. The N-terminal VH domain from the first heavy chain polypeptide and the VL domain from the light chain polypeptide form an antigen binding site specifically binding to a target antigen. The heavy chain polypeptides of the first heterodimeric precursor polypeptide are associated with each other via their CH3 domains. No interchain disulfide bond is formed between the first and second heavy chain polypeptide. A pair of a VH domain and a VL domain is formed between the VH domain derived from the first antibody and the VL domain from the second heavy chain polypeptide. Both variable domains are associated with each other, but do not form an antigen binding site specifically binding to an antigen. A second heterodimeric precursor polypeptide comprises three polypeptide chains: 1. A first heavy chain polypeptide comprising from N- to C-terminal direction antibody domains VH, CH1, peptide connector, a VL domain derived from the first antibody, CH2 and CH3. The CH3 domain comprises a hole mutation and does not comprise a destabilizing mutation. 2. A second heavy chain polypeptide comprising from N- to C-terminal direction the following antibody domains: a VH domain derived from a third antibody, CH2 and CH3. The CH3 domain comprises a knob mutation and a destabilizing mutation. 3. A light chain polypeptide comprising from N- to C-terminal direction antibody domains VL and CL. The N-terminal VH domain of the first heavy chain polypeptide and the VL domain from the light chin polypeptide form an antigen binding site specifically binding to a target antigen. The heavy chain polypeptides of the second heterodimeric precursor polypeptide are associated with each other via their CH3 domains. No interchain disulfide bond is formed between the first and second heavy chain polypeptide. A pair of a VH domain and a VL domain is formed between the VL domain derived from the first antibody and the VH domain from the second heavy chain polypeptide. Both variable domains are associated with each other, but do not form an antigen binding site specifically binding to an antigen. Upon polypeptide chain exchange, heterodimeric product polypeptides are formed. A first product polypeptide comprises the two antigen binding sites from the precursor polypeptides, i.e. the antigen binding site from the first heterodimeric precursor polypeptide and the antigen binding site from the second heterodimeric precursor polypeptide. The first product polypeptide comprises the first heavy chain polypeptides from the first and second heterodimeric precursor polypeptides, which are associated via their CH3 domains. Both heavy chain polypeptides comprised in the first product polypeptide comprise CH3 domains that do not comprise destabilizing mutations. By association of the first heavy chain polypeptides from the first and second heterodimeric precursor polypeptides a pair of a VH domain derived from a first antibody and a VL domain derived from a first antibody are formed, which form an antigen binding site specifically binding to a first antigen. This antigen binding site does not exist in any precursor polypeptide and is only formed (activated) upon polypeptide chain exchange. The second product polypeptide comprises the second heavy chain polypeptide from the first heterodimeric precursor polypeptide and the second heavy chain polypeptide from the second heterodimeric precursor polypeptide. Both heavy chain polypeptides are associated via their CH3 domains. Both CH3 domains comprise destabilizing mutations, which interact and support the formation of the heterodimeric product polypeptide. By association of the second heavy chain polypeptides from the first and second heterodimeric precursor polypeptides a new pair of a VH domain and a VL domain is formed. Both variable domains are associated in the second product polypeptide.

FIG. 4: Exemplary domain arrangements of first heterodimeric precursor polypeptides. Depiction of knobs-into-holes mutations, destabilizing mutations and cysteine mutations is the same as in FIGS. 1 to 3. Precursor polypeptides may comprise one or more antigen binding sites, which may be arranged C-terminally or N-terminally. While the images indicate precursor polypeptides comprising Fab fragments, it is understood that the precursor polypeptides may comprise other appropriate antigen binding moieties. Depiction of hinge region disulfide bonds is exemplary, depending on the desired application, hinge region disulfide bonds may or may not be present. Depiction of cysteine mutations is exemplary, but not mandatory in precursor polypeptides of the invention. A) Precursor polypeptide comprising an N-terminal Fab fragment and a CH2 domain. B) Precursor polypeptide comprising a C-terminal and an N-terminal Fab fragment and a CH2 domain. C) Precursor polypeptide comprising a C-terminal Fab fragment and a CH2 domain. D) Precursor polypeptide with activatable binding site comprising a N-terminal Fab fragment and a VH domain. E) Precursor polypeptide with activatable binding site comprising a C-terminal and an N-terminal Fab fragment and a VH domain. F) Precursor polypeptide with activatable binding site comprising a C-terminal Fab fragment and a VH domain. G) Precursor polypeptide with activatable binding site comprising a CH2 domain comprising an N-terminal Fab fragment and a VH domain. H) Precursor polypeptide with activatable binding site comprising a CH2 domain comprising an N-terminal and a C-terminal Fab fragment and a VH domain. I) Precursor polypeptide with activatable binding site comprising a CH2 domain comprising a C-terminal Fab fragment and a VH domain. J) Precursor polypeptide with C-terminal activatable binding site comprising a CH2 domain comprising an N-terminal Fab fragment and a VH domain. K) Precursor polypeptide with activatable binding site comprising a N-terminal Fab fragment and a VL domain.

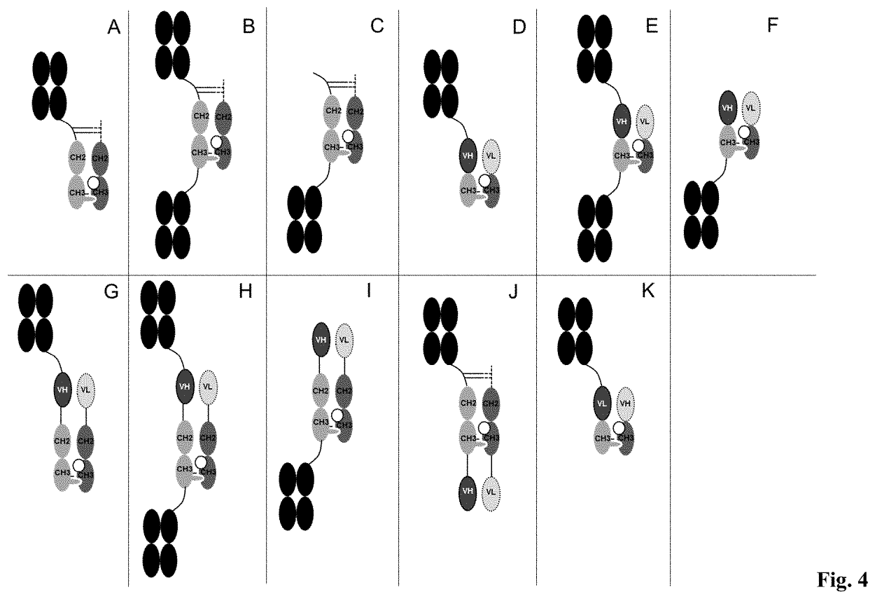

FIG. 5: Exemplary domain arrangements of second heterodimeric precursor polypeptides. Depiction of knobs-into-holes mutations, destabilizing mutations and cysteine mutations is the same as in FIGS. 1 to 3. Precursor polypeptides may comprise one or more antigen binding sites, which may be arranged C-terminally or N-terminally. While the images indicate precursor polypeptides comprising Fab fragments, it is understood that the precursor polypeptides may comprise other appropriate antigen binding moieties. Depiction of hinge region disulfide bonds is exemplary, depending on the desired application, hinge region disulfide bonds may or may not be present. Depiction of cysteine mutations is exemplary, but not mandatory in precursor polypeptides of the invention. A) Precursor polypeptide comprising an N-terminal Fab fragment and a CH2 domain. B) Precursor polypeptide comprising a C-terminal and an N-terminal Fab fragment and a CH2 domain. C) Precursor polypeptide comprising a C-terminal Fab fragment and a CH2 domain. D) Precursor polypeptide with activatable binding site comprising a N-terminal Fab fragment and a VL domain. E) Precursor polypeptide with activatable binding site comprising a C-terminal and an N-terminal Fab fragment and a VL domain. F) Precursor polypeptide with activatable binding site comprising a C-terminal Fab fragment and a VL domain. G) Precursor polypeptide with activatable binding site comprising a CH2 domain comprising an N-terminal Fab fragment and a VL domain. H) Precursor polypeptide with activatable binding site comprising a CH2 domain comprising an N-terminal and a C-terminal Fab fragment and a VL domain. I) Precursor polypeptide with activatable binding site comprising a CH2 domain comprising a C-terminal Fab fragment and a VL domain. J) Precursor polypeptide with C-terminal activatable binding site comprising a CH2 domain comprising an N-terminal Fab fragment and a VL domain. K) Precursor polypeptide with activatable binding site comprising a N-terminal Fab fragment and a VH domain.

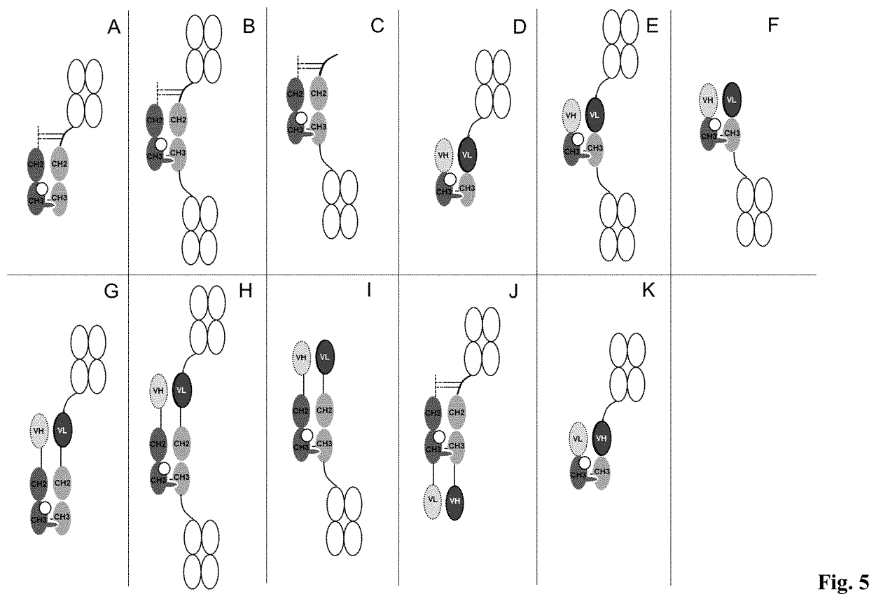

### DETAILED DESCRIPTION OF THE INVENTION

### 1. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

The terms “a”, “an” and “the” generally include plural referents, unless the context clearly indicates otherwise.

Unless otherwise defined herein the term “comprising of” shall include the term “consisting of”.

The provision of two alternatives using the terms “either . . . or” designates mutually exclusive alternatives, unless the context clearly indicates otherwise.

The term “antigen binding moiety” as used herein refers to a moiety that specifically binds to a target antigen. The term includes antibodies as well as other natural (e.g. receptors, ligands) or synthetic (e.g. DARPins) molecules capable of specifically binding to a target antigen.

The term “antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “binding site” or “antigen-binding site” as used herein denotes the region or regions of an antigen binding moiety to which the antigen actually binds. In case the antigen binding moiety is an antibody, the antigen-binding site includes antibody heavy chain variable domains (VH) and/or antibody light chain variable domains (VL), or pairs of VH/VL. Antigen-binding sites derived from antibodies that specifically bind to a target antigen can be derived a) from known antibodies specifically binding to the antigen or b) from new antibodies or antibody fragments obtained by de novo immunization methods using inter alia either the antigen protein or nucleic acid or fragments thereof or by phage display methods.

When being derived from an antibody, an antigen-binding site of an antibody according to the invention can contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences. Also included within the scope of the invention are functional antigen binding sites comprised of fewer CDRs (i.e., where binding specificity is determined by three, four or five CDRs). For example, less than a complete set of 6 CDRs may be sufficient for binding.

The term “valent” as used herein denotes the presence of a specified number of binding sites in an antibody molecule. A natural antibody for example has two binding sites and is bivalent. As such, the term “trivalent” denotes the presence of three binding sites in an antibody molecule.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv, scFab); and multispecific antibodies formed from antibody fragments.

“Specificity” refers to selective recognition of a particular epitope of an antigen by the antigen binding moiety, e.g. an antibody. Natural antibodies, for example, are monospecific. The term “monospecific antibody” as used herein denotes an antibody that has one or more binding sites each of which bind to the same epitope of the same antigen. “Multispecific antibodies” bind two or more different epitopes (for example, two, three, four, or more different epitopes). The epitopes may be on the same or different antigens. An example of a multispecific antibody is a “bispecific antibody” which binds two different epitopes. When an antibody possesses more than one specificity, the recognized epitopes may be associated with a single antigen or with more than one antigen.

An epitope is a region of an antigen that is bound by an antigen binding moiety, e.g. an antibody. The term “epitope” includes any polypeptide determinant capable of specific binding to an antibody or antigen binding moiety. In certain embodiments, epitope determinants include chemically active surface groupings of molecules such as amino acids, glycan side chains, phosphoryl, or sulfonyl, and, in certain embodiments, may have specific three dimensional structural characteristics, and/or specific charge characteristics.

As used herein, the terms “binding” and “specific binding” refer to the binding of the antibody or antigen binding moiety to an epitope of the antigen in an in vitro assay, preferably in a plasmon resonance assay (BIAcore®, GE-Healthcare Uppsala, Sweden) with purified wild-type antigen. In certain embodiments, an antibody or antigen binding moiety is said to specifically bind an antigen when it preferentially recognizes its target antigen in a complex mixture of proteins and/or macromolecules.

The affinity of the binding of an antibody to an antigen is defined by the terms kₐ (rate constant for the association of the antibody from the antibody/antigen complex), k_{D} (dissociation constant), and K_{D} (k_{D}/ka). In one embodiment binding or that/which specifically binds to means a binding affinity (K_{D}) of 10⁸ mol/l or less, in one embodiment 10⁻⁸ M to 10⁻¹³ mol/l. Thus, an antigen binding moiety, particularly an antibody binding site, specifically binds to each antigen for which it is specific with a binding affinity (K_{D}) of 10⁻⁸ mol/l or less, e.g. with a binding affinity (K_{D}) of 10⁻⁸ to 10⁻¹³ mol/l. in one embodiment with a binding affinity (K_{D}) of 10⁻⁹ to 10⁻¹³ mol/l.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “constant domains” or “constant region” as used within the current application denotes the sum of the domains of an antibody other than the variable region. The constant region is not directly involved in binding of an antigen, but exhibits various effector functions.

Depending on the amino acid sequence of the constant region of their heavy chains, antibodies are divided in the “classes”: IgA, IgD, IgE, IgG and IgM, and several of these may are further divided into subclasses, such as IgG1, IgG2, IgG3, and IgG4, IgA1 and IgA2. The heavy chain constant regions that correspond to the different classes of antibodies are called α, δ, ε, γ and μ, respectively. The light chain constant regions (CL) which can be found in all five antibody classes are called κ (kappa) and λ (lambda).

The “constant domains” as used herein are, preferably, from human origin, which is from a constant heavy chain region of a human antibody of the subclass IgG1, IgG2, IgG3, or IgG4 and/or a constant light chain kappa or lambda region. Such constant domains and regions are well known in the state of the art and e.g. described by Kabat, et al., Sequences of Proteins of Immunological Interest, 5th ed., Public Health Service, National Institutes of Health, Bethesda, Md. (1991).

In wild type antibodies, the “hinge region” is a flexible amino acid stretch in the central part of the heavy chains of the IgG and IgA immunoglobulin classes, which links the two heavy chains by disulfide bonds, i.e. “interchain disulfide bonds” as they are formed between the two heavy chains. The hinge region of human IgG1 is generally defined as stretching from about Glu216, or about Cys226, to about Pro230 of human IgG1 (Burton, Molec. Immunol. 22:161-206 (1985)). By deleting cysteine residues in the hinge region or by substituting cysteine residues in the hinge region by other amino acids, such as serine, disulfide bond formation in the hinge region is avoided.

The “light chains” of antibodies from any vertebrate species can be assigned to one of two distinct types, called kappa (x) and lambda (k), based on the amino acid sequences of their constant domains. A wild type light chain typically contains two immunoglobulin domains, usually one variable domain (VL) that is important for binding to an antigen and a constant domain (CL).

Several different types of “heavy chains” exist that define the class or isotype of an antibody. A wild type heavy chain contains a series of immunoglobulin domains, usually with one variable domain (VH) that is important for binding antigen and several constant domains (CH1, CH2, CH3, etc.).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “CH2 domain” of a human IgG Fc region usually extends from an amino acid residue at about position 231 to an amino acid residue at about position 340. The multispecific antibody is devoid of a CH2 domain. By “devoid of a CH2 domain” is meant that the antibodies according to the invention do not comprise a CH2 domain.

The “CH3 domain” comprises the stretch of residues C-terminal to a CH2 domain in an Fc region (i.e. from an amino acid residue at about position 341 to an amino acid residue at about position 447 of an IgG). The “CH3 domains” herein are variant CH3 domains, wherein the amino acid sequence of the natural CH3 domain was subjected to at least one distinct amino acid substitution (i.e. modification of the amino acid sequence of the CH3 domain) in order to promote heterodimerization of the two CH3 domains facing each other within the multispecific antibody.

Typically, in the heterodimerization approaches known in the art, the CH3 domain of one heavy chain and the CH3 domain of the other heavy chain are both engineered in a complementary manner so that the heavy chain comprising one engineered CH3 domain can no longer homodimerize with another heavy chain of the same structure. Thereby the heavy chain comprising one engineered CH3 domain is forced to heterodimerize with the other heavy chain comprising the CH3 domain, which is engineered in a complementary manner.

One heterodimerization approach known in the art is the so-called “knobs-into-holes” technology, which is described in detail providing several examples in e.g. WO 96/027011, Ridgway, J. B., et al., Protein Eng. 9 (1996) 617-621; Merchant, A. M., et al., Nat. Biotechnol. 16 (1998) 677-681; and WO 98/050431, which are herein included by reference. In the “knobs-into-holes” technology, within the interface formed between two CH3 domains in the tertiary structure of the antibody, particular amino acids on each CH3 domain are engineered to produce a protuberance (“knob”) in one of the CH3 domains and a cavity (“hole”) in the other one of the CH3 domains, respectively. In the tertiary structure of the multispecific antibody the introduced protuberance in the one CH3 domain is positionable in the introduced cavity in the other CH3 domain.

In combination with the substitutions according to the knobs-into-holes technology, additional interchain disulfide bonds may be introduced into the CH3 domains to further stabilize the heterodimerized polypeptides (Merchant, A. M., et al., Nature Biotech. 16 (1998) 677-681). Such interchain disulfide bonds are formed, e.g. by introducing the following amino acid substitutions into the CH3 domains: D399C in one CH3 domain and K392C in the other CH3 domain; Y349C in one CH3 domain and S354C in the other CH3 domain; Y349C in one CH3 domain and E356C in the other CH3 domain; Y349C in one CH3 domain and E357C in the other CH3 domain; L351C in one CH3 domain and S354C in the other CH3 domain; T394C in one CH3 domain and V397C in the other CH3 domain. A “cysteine mutation” as used herein refers to one amino acid substitution of an amino acid in a CH3 domain by cysteine that is capable of forming an interchain disulfide bond with another, matching, amino acid substitution of an amino acid in a second CH3 domain by cysteine.

Further techniques, apart from the “knobs-into-holes” technology as mentioned before, for modifying the CH3 domains in order to enforce heterodimerization are known in the art. These technologies, especially the ones described in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291 are contemplated herein as alternatives to the “knobs-into-holes technology” for the polypeptides provided by the invention. All those technologies involve engineering of CH3 domains in a complementary manner, by introduction of amino acids of opposite charge or different side chain volume, thereby supporting heterodimerization.

Precursor polypeptides of the invention comprise in only one of their CH3 domains an amino acid substitution “destabilizing the CH3/CH3 interface”, also referred to herein as “destabilizing mutations”. With these termini, amino acid substitutions are meant that are arranged in only one of the CH3 domains that are associated in the heterodimeric precursor polypeptide. In said CH3 domain, one or more amino acid positions known to interact within the CH3/CH3 interface, e.g. as disclosed in the prior art related to CH3-heterodimerization strategies indicated above, is replaced by an amino acid with another site-chain property. In contrast to heterodimerization strategies, wherein typically a pair of interacting amino acids in the associated CH3 domains is substituted (i.e. one or more amino acid residues in one CH3 domain involved in the heterodimer; and one or more amino acid residues in the other CH3 domain involved in the heterodimer), the destabilizing mutation is arranged in only one of the CH3 domains involved in the heterodimeric precursor polypeptides according to the invention. Exemplary amino acid substitutions destabilizing the CH3/CH3 interface are listed below in the section “Destabilizing mutations”. All exemplary amino acid substitutions specifically disclosed herein are arranged such that the substituted amino acids interact in the CH3/CH3 interface within a pair of said CH3 domains.

The term “polypeptide chain” as used herein refers to a linear organic polymer comprising a large number of amino acids linked together via peptide bonds. One or more polypeptide chains form a “polypeptide” or “protein”, wherein both terms are used interchangeably herein. Heterodimeric precursor polypeptides as provided in a set according to the invention comprise at least two polypeptide chains comprising a CH3 domain. Thus, a first polypeptide chain comprising a first CH3 domain is “associated” with a second polypeptide chain comprising a second CH3 domain to form a dimeric polypeptide. As the first CH3 domain and the second CH3 domain comprise amino acid substitutions according to the knobs-into-holes technology, the two polypeptide chains form a “heterodimer”, i.e. a dimer formed by two non-identical polypeptides.

The polypeptide chains comprised in the heterodimeric polypeptides, i.e. the heterodimeric precursor polypeptides and the heterodimeric product polypeptides, comprise one or two polypeptide domains. When the order of the polypeptide domains is indicated herein, it is indicated in N- to C-terminal direction.

Each heterodimeric precursor polypeptide comprises at least two polypeptide chains comprising a CH3 domain.

In case the antigen binding moiety present in the two heterodimeric precursor polypeptides are antibody-derived antigen binding sites, e.g. antibody fragments, the polypeptide chain comprising the CH3 domain is herein also referred to as “heavy chain polypeptide”. In this case, the heterodimeric precursor polypeptide may also comprise a “light chain polypeptide”, typically comprising an antibody variable domain and an antibody constant domain, e.g. VL and CL.

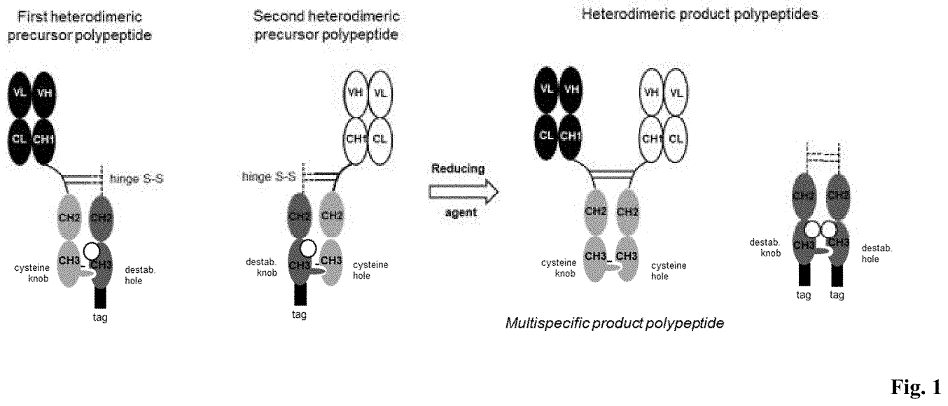

The invention provides a set comprising at least two polypeptides. The set comprises at least two heterodimeric “precursor” polypeptides. When reacting the precursor polypeptides to undergo a polypeptide chain exchange with each other, “product” polypeptides are formed. The invention also provides a method for generating a heterodimeric polypeptide, i.e. a heterodimeric product polypeptide, by contacting at least two heterodimeric precursor polypeptides. The step of contacting may be carried out in any appropriate allowing the polypeptide chain exchange, preferably in an appropriate buffer solution. By “polypeptide chain exchange” when referred to herein in connection with the invention is meant the exchange of a polypeptide chain comprising a CH3 domain between two heterodimeric (precursor) polypeptides. A polypeptide chain exchange occurs, when the two, initially associated, polypeptide chains comprising a CH3 domain from a precursor polypeptide dissociate and at least one of the dissociated polypeptide chains forms a new heterodimer by association with an, equally dissociated, polypeptide chain comprising a CH3 domain derived from another precursor polypeptide. The mechanism of polypeptide chain exchange is also indicated in FIGS. 1, 2 and 3.

An “isolated” heterodimeric polypeptide, e.g. an antibody, is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991). In particular, for variable domains and for the light chain constant domain CL of kappa and lambda isotype, the Kabat numbering system (see pages 647-660) of Kabat, et al., Sequences of Proteins of Immunological Interest, 5th ed., Public Health Service, National Institutes of Health, Bethesda, Md. (1991) is used and for the constant heavy chain domains (CH1, Hinge, CH2 and CH3) the Kabat EU index numbering system (see pages 661-723) is used. Amino acid positions provided herein are usually indicated by

Amino acid “substitutions” or “replacements” or “mutations” (all terms are herein used interchangeably) within the polypeptide chains are prepared by introducing appropriate nucleotide changes into the antibody DNA, or by nucleotide synthesis. Such modifications can be performed, however, only in a very limited range. For example, the modifications do not alter the above mentioned antibody characteristics such as the IgG isotype and antigen binding, but may further improve the yield of the recombinant production, protein stability or facilitate the purification. In certain embodiments, antibody variants having one or more conservative amino acid substitutions are provided. A “double mutation” as referred herein means that both of the indicated amino acid substitutions are present in the respective polypeptide chain.

The term “amino acid” as used herein denotes an organic molecule possessing an amino moiety located at α-position to a carboxylic group. Examples of amino acids include: arginine, glycine, omithine, lysine, histidine, glutamic acid, asparagic acid, isoleucine, leucine, alanine, phenylalanine, tyrosine, tryptophane, methionine, serine, proline. The amino acid employed is optionally in each case the L-form. The term “positively charged” or “negatively charged” amino acid refers to the amino acid side-chain charge at pH 7.4. Amino acids may be grouped according to common side-chain properties:
(1) hydrophobic: Norleucine, Met, Ala, Val, Leu, Ile, Trp, Tyr, Phe;
(2) neutral hydrophilic: Cys, Ser, Thr, Asn, Gln;
(3) acidic or negatively charged: Asp, Glu;
(4) basic or positively charged: His, Lys, Arg;
(5) residues that influence chain orientation: Gly, Pro;

TABLE
Amino acids with specific properties

| | | 1- | Side-chain | Side-chain charge |
|---|---|---|---|---|
| Amino Acid | 3-Letter | Letter | polarity | (pH 7.4) |
| Alanine | Ala | A | nonpolar | neutral |
| Arginine | Arg | R | basic polar | positive |
| Asparagine | Asn | N | polar | neutral |
| Aspartic acid | Asp | D | acidic polar | negative |
| Cysteine | Cys | C | nonpolar | neutral |
| Glutamic acid | Glu | E | acidic polar | negative |
| Glutamine | Gln | Q | polar | neutral |
| Glycine | Gly | G | nonpolar | neutral |
| Histidine | His | H | basic polar | positive (10%) |
| | | | | neutral (90%) |
| Isoleucine | Ile | I | nonpolar | neutral |
| Leucine | Leu | L | nonpolar | neutral |
| Lysine | Lys | K | basic polar | positive |
| Methionine | Met | M | nonpolar | neutral |
| Phenylalanine | Phe | F | nonpolar | neutral |
| Proline | Pro | P | nonpolar | neutral |
| Serine | Ser | S | polar | neutral |
| Threonine | Thr | T | polar | neutral |
| Tryptophan | Trp | W | nonpolar | neutral |
| Tyrosine | Tyr | Y | polar | neutral |
| Valine | Val | V | nonpolar | neutral |

As used herein a “tagging moiety” is a peptide sequence genetically grafted onto the polypeptide chain for various purposes, e.g. to support purification. In one embodiment the tagging moiety is an affinity tag. Thus a polypeptide comprising said affinity tag may be purified via an appropriate affinity technique, e.g. by affinity chromatography. Typically, the tagging moiety is fused to the C-terminus of the CH3 domains via a peptide connector. Typically, the peptide connectors are composed of flexible amino acid residues like glycine and serine. Thus, typical peptide connectors used for fusing tagging moieties to polypeptides are glycine-serine linkers, i.e. peptide connectors consisting of a pattern of glycine and serine residues.

The term “purified” as used herein refers to polypeptides, that are removed from their natural environment or from a source of recombinant production, or otherwise isolated or separated, and are at least 60%, e.g., at least 80%, free from other components, e.g. membranes and microsomes, with which they are naturally associated. Purification of antibodies (recovering the antibodies from the host cell culture) is performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987). Different methods are well established and widespread used for protein purification, such as affinity chromatography with microbial proteins (e.g. with affinity media for the purification of kappa or lambda-isotype constant light chain domains, e.g. KappaSelect or LambdaSelect), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretic methods (such as gel electrophoresis, capillary electrophoresis) (Vijayalakshmi, M. A., Appl. Biochem. Biotech. 75 (1998) 93-102).

Polypeptides comprising tagging moieties may be purified via “tag-specific affinity chromatography”. Appropriate purification methods for tags are known in the art. Thus, polypeptides comprising a poly(his) tag may be purified, e.g. via metal chelate affinity chromatography, particularly nickel chelate affinity chromatography.

The term “peptide connector” as used herein denotes a peptide with amino acid sequences, which is preferably of synthetic origin. Within heterodimeric polypeptides as used for the invention, peptide connectors may be used for fusing additional polypeptide domains, like antibody fragments, to the C- or N-terminus of an individual polypeptide chain. In one embodiment said peptide connectors are peptides with an amino acid sequence with a length of at least 5 amino acids, in another embodiment with a length of 5 to 100 amino acids, in yet another embodiment of 10 to 50 amino acids. In one embodiment the peptide connector is a glycine-serine linker. In one embodiment the peptide connector is a peptide consisting of glycine and serine amino acid residues. In one embodiment said peptide connector is

(GₓS)ₙ or(GₓS)ₙGₘ

with G=glycine, S=serine, and

x=3, n=3, 4, 5 or 6, m=0, 1, 2 or 3; or

x=4, n=2, 3, 4 or 5, m=0, 1, 2 or 3.

In one embodiment x=4 and n=2 or 3, in another embodiment x=4, n=2.

In one embodiment said peptide connector is (G₄S)₂.

The term “valent” as used herein denotes the presence of a specified number of binding sites in an antigen binding molecule. A natural antibody for example has two binding sites and is bivalent. As such, the term “trivalent” denotes the presence of three binding sites in an antigen binding molecule.

Polypeptides according to the invention are produced by recombinant means. Methods for recombinant production of polypeptides, e.g. antibodies, are widely known in the state of the art and comprise protein expression in prokaryotic and eukaryotic host cells with subsequent isolation of the polypeptide and usually purification to a pharmaceutically acceptable purity. For the expression of the polypeptides as aforementioned in a host cell, nucleic acids encoding the respective polypeptide chains are inserted into expression vectors by standard methods. Expression is performed in appropriate prokaryotic or eukaryotic host cells, like CHO cells, NS0 cells, SP2/0 cells, HEK293 cells, COS cells, PER.C6 cells, yeast, or E. coli cells, and the polypeptide is recovered from the cells (supernatant or cells after lysis). General methods for recombinant production of polypeptides, e.g. antibodies, are well-known in the state of the art and described, for example, in the review articles of Makrides, S. C., Protein Expr. Purif. 17 (1999) 183-202; Geisse, S., et al., Protein Expr. Purif 8 (1996) 271-282; Kaufman, R. J., Mol. Biotechnol. 16 (2000) 151-161; Werner, R. G., Drug Res. 48 (1998) 870-880.

Polypeptides produced by host cells may undergo post-translational cleavage of one or more, particularly one or two, amino acids from the C-terminus of the polypeptide chain comprising a CH3 domain at the C-terminal end. Therefore, a polypeptide produced by a host cell by expression of a specific nucleic acid molecule encoding such polypeptide chain may include the full-length polypeptide chain including the full length CH3 domain, or it may include a cleaved variant of the full-length polypeptide chain (also referred to herein as a cleaved variant polypeptide chain). This may be the case where the final two C-terminal amino acids of the heavy chain are glycine (G446) and lysine (K447).

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a heterodimeric polypeptide” refers to one or more nucleic acid molecules encoding one or more polypeptide chains (or fragments thereof) of said heterodimeric polypeptide, including such nucleic acid molecule(s) in a single vector or separate vectors, and such nucleic acid molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. The term includes vectors that function primarily for insertion of DNA or RNA into a cell (e.g., chromosomal integration), replication of vectors that function primarily for the replication of DNA or RNA, and expression vectors that function for transcription and/or translation of the DNA or RNA. Also included are vectors that provide more than one of the functions as described.

An “expression vector” is a vector are capable of directing the expression of nucleic acids to which they are operatively linked. When the expression vector is introduced into an appropriate host cell, it can be transcribed and translated into a polypeptide. When transforming host cells in methods according to the invention, “expression vectors” are used; thereby the term “vector” in connection with transformation of host cells as described herein means “expression vector”. An “expression system” usually refers to a suitable host cell comprised of an expression vector that can function to yield a desired expression product.

As used herein, “expression” refers to the process by which a nucleic acid is transcribed into mRNA and/or to the process by which the transcribed mRNA (also referred to as a transcript) is subsequently translated into a peptide or polypeptide. The transcripts and the encoded polypeptides are individually or collectively referred to as gene products. If a nucleic acid is derived from genomic DNA, expression in a eukaryotic cell may include splicing of the corresponding mRNA.

The term “transformation” as used herein refers to process of transfer of a vector or a nucleic acid into a host cell. If cells without formidable cell wall barriers are used as host cells, transfection is carried out e.g. by the calcium phosphate precipitation method as described by Graham and Van der Eh, Virology 52 (1978) 546ff. However, other methods for introducing DNA into cells such as by nuclear injection or by protoplast fusion may also be used. If prokaryotic cells or cells which contain substantial cell wall constructions are used, e.g. one method of transfection is calcium treatment using calcium chloride as described by Cohen, F. N, et al., PNAS 69 (1972) 7110 et seq.

The term “host cell” as used in the current application denotes any kind of cellular system which can be engineered to generate the polypeptides provided with the invention.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; and Norderhaug, L., et al., J. Immunol. Methods 204 (1997) 77-87. A preferred transient expression system (HEK 293) is described by Schlaeger, E.-J., and Christensen, K., in Cytotechnology 30 (1999) 71-83 and by Schlaeger, E.-J., J. Immunol. Methods 194 (1996) 191-199.

The term “pharmaceutical composition” refers to a preparation which is in such form as to permit the biological activity of an active ingredient contained therein to be effective, and which contains no additional components which are unacceptably toxic to a subject to which the composition would be administered. A pharmaceutical composition of the present invention can be administered by a variety of methods known in the art. As will be appreciated by the skilled artisan, the route and/or mode of administration will vary depending upon the desired results. To administer an antibody according to the invention by certain routes of administration, it may be necessary to coat the antibody with, or co-administer the antibody with, a material to prevent its inactivation. For example, the heterodimeric polypeptide may be administered to a subject in an appropriate carrier, for example, liposomes, or a diluent. Pharmaceutically acceptable diluents include saline and aqueous buffer solutions.

A pharmaceutical composition comprises an effective amount of the heterodimeric polypeptides provided with the invention. An “effective amount” of an agent, e.g., a heterodimeric polypeptide, refers to an amount effective, at dosages and for periods of time necessary, to achieve the desired therapeutic or prophylactic result. In particular, the “effective amount” denotes an amount of a heterodimeric polypeptide of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the heterodimeric polypeptide molecules used, disease state being treated, the severity or the disease treated, the age and relative health of the subject, the route and form of administration, the judgment of the attending medical or veterinary practitioner, and other factors.

A “pharmaceutically acceptable carrier” refers to an ingredient in a pharmaceutical formulation, other than an active ingredient, which is nontoxic to a subject. Pharmaceutically acceptable carriers include any and all solvents, dispersion media, coatings, antibacterial and antifungal agents, isotonic and absorption delaying agents, and the like that are physiologically compatible. In one preferred embodiment, the carrier is suitable for intravenous, intramuscular, subcutaneous, parenteral, spinal or epidermal administration (e.g. by injection or infusion).

The pharmaceutical compositions according to the invention may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The composition must be sterile and fluid to the extent that the composition is deliverable by syringe. In addition to water, in one embodiment the carrier is an isotonic buffered saline solution.

Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

### 2. Detailed Description of the Embodiments of the Invention

The invention provides precursor polypeptides applicable, e.g. for in vitro generation of product polypeptides by polypeptide chain exchange. Each precursor polypeptide comprises a pair of CH3 domains arranged on two individual polypeptide chains that are associated with each other via said CH3 domains. Said CH3 domains comprise several amino acid substitutions. Due to this, the two polypeptide chains comprising the CH3 domains in the precursor polypeptide form a heterodimer. The CH3 domains of the precursor polypeptides provided by the invention comprise at least two patterns of mutations, with different functionalities. The first pattern of mutations are mutations supporting the heterodimerization of said two polypeptide chains comprising the CH3 domains, i.e. knobs-into-holes mutations. Thus, one CH3 domain of a precursor polypeptide comprises a knob mutation and the other CH3 domain of the precursor polypeptide comprises a hole mutation. The second pattern of mutations are one or more mutations provided in only one of the CH3 domains involved in the heterodimer of a precursor polypeptide, wherein said mutation destabilizes the interaction of the two polypeptides comprising the CH3 domains. Thus, each precursor polypeptide comprises one CH3 domain with a destabilizing mutations, which is selected and arranged such that they support correct assembly of the product polypeptide upon polypeptide chain exchange between the precursor polypeptides.

### Precursor Polypeptides

In one aspect the invention provides a set of heterodimeric precursor polypeptides comprising:
a) a first heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation,
   wherein the first heterodimeric precursor polypeptide comprises a first antigen binding moiety, wherein at least a part of the first antigen binding moiety is arranged on one of the two polypeptide chains comprising the CH3 domain, and
b) a second heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation,
   wherein the second heterodimeric precursor polypeptide comprises a second antigen binding moiety, wherein at least a part of the second antigen binding moiety is arranged on one of the two polypeptide chains comprising the CH3 domain,
wherein
A) either i) within the first heterodimeric precursor polypeptide the polypeptide chain comprising the CH3 domain comprising the knob mutation comprises at least a part of the first antigen binding moiety and within the second heterodimeric precursor polypeptide the polypeptide chain comprising the CH3 domain with the hole mutation comprises at least a part of the second antigen binding moiety, or ii) within the first heterodimeric precursor polypeptide the polypeptide chain comprising the CH3 domain comprising the hole mutation comprises at least a part of the first antigen binding moiety and within the second heterodimeric precursor polypeptide the polypeptide chain comprising the CH3 domain with the knob mutation comprises at least a part of the second antigen binding moiety; and wherein
B) either
   i) the CH3 domain of the first heterodimeric precursor polypeptide comprising the knob mutation and the CH3 domain of the second heterodimeric precursor polypeptide comprising the hole mutation, or
   ii) the CH3 domain of the first heterodimeric precursor polypeptide comprising the hole mutation and the CH3 domain of the second heterodimeric precursor polypeptide comprising the knob mutation
   comprises an amino acid substitution destabilizing the CH3/CH3 interface, wherein the amino acid substitutions are arranged such that the substituted amino acids interact in the CH3/CH3 interface within a pair of said CH3 domains.

In one aspect the invention provides a set of heterodimeric precursor polypeptides comprising:
a) a first heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation,
   wherein the first heterodimeric precursor polypeptide comprises a first antigen binding moiety, wherein at least a part of the first antigen binding moiety is arranged on one of the two polypeptide chains comprising the CH3 domain, and
b) a second heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation,
   wherein the second heterodimeric precursor polypeptide comprises a second antigen binding moiety, wherein at least a part of the second antigen binding moiety is arranged on one of the two polypeptide chains comprising the CH3 domain,
wherein
A) either i) within the first heterodimeric precursor polypeptide the polypeptide chain comprising the CH3 domain comprising the knob mutation comprises at least a part of the first antigen binding moiety and within the second heterodimeric precursor polypeptide the polypeptide chain comprising the CH3 domain with the hole mutation comprises at least a part of the second antigen binding moiety, or ii) within the first heterodimeric precursor polypeptide the polypeptide chain comprising the CH3 domain comprising the hole mutation comprises at least a part of the first antigen binding moiety and within the second heterodimeric precursor polypeptide the polypeptide chain comprising the CH3 domain with the knob mutation comprises at least a part of the second antigen binding moiety; and wherein
B) either
   i) the CH3 domain of the first heterodimeric precursor polypeptide comprising the knob mutation and the CH3 domain of the second heterodimeric precursor polypeptide comprising the hole mutation, or
   ii) the CH3 domain of the first heterodimeric precursor polypeptide comprising the hole mutation and the CH3 domain of the second heterodimeric precursor polypeptide comprising the knob mutation comprise the following amino acid substitutions (which are also referred to herein as “destabilizing mutations”), wherein the numbering is according to the Kabat numbering system:
      the CH3 domain with the hole mutation comprises at least one amino acid substitution selected from the group of:
         replacement of S354 with a hydrophobic amino acid;
         replacement of D356 with a positively charged amino acid;
         replacement of E357 with a positively charged amino acid or with a hydrophobic amino acid;
         replacement of D356 with a positively charged amino acid, and replacement of E357 with a positively charged amino acid or with a hydrophobic amino acid;
         replacement of S364 with a hydrophobic amino acid;
         replacement of A368 with a hydrophobic amino acid;
         replacement of E392 with a negatively charged amino acid;
         replacement of T394 with a hydrophobic amino acid;
         replacement of D399 with a hydrophobic amino acid and replacement of S400 with a positively charged amino acid;
         replacement of D399 with a hydrophobic amino acid and replacement of F405 with a positively charged amino acid;
         replacement of V407 with a hydrophobic amino acid; and
         replacement of K409 with a negatively charged amino acid; and
         replacement of K439 with a negatively charged amino acid;
      the CH3 domain with the knob mutation comprises at least one amino acid substitution selected from the group of:
         replacement of Q347 with a positively charged amino acid, and replacement of K360 with a negatively charged amino acid;
         replacement of Y349 with a negatively charged amino acid;
         replacement of L351 with a hydrophobic amino acid, and replacement of E357 with a hydrophobic amino acid;
         replacement of S364 with a hydrophobic amino acid;
         replacement of W366 with a hydrophobic amino acid, and replacement of K409 with a negatively charged amino acid;
         replacement of L368 with a hydrophobic amino acid;
         replacement of K370 with a negatively charged amino acid;
         replacement of K370 with a negatively charged amino acid, and replacement of K439 with a negatively charged amino acid;
         replacement of K392 with a negatively charged amino acid;
         replacement of T394 with a hydrophobic amino acid;
         replacement of V397 with a hydrophobic amino acid;
         replacement of D399 with a positively charged amino acid, and replacement of K409 with a negatively charged amino acid;
         replacement of S400 with a positively charged amino acid;
         F405W;
         Y407W; and
         replacement of K439 with a negatively charged amino acid.

In another aspect the invention provides a first heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation, wherein the first heterodimeric precursor polypeptide comprises a first antigen binding moiety, wherein at least a part of the first antigen binding moiety is arranged on one of the two polypeptide chains comprising the CH3 domain; and wherein one of the CH3 domains (but not the other CH3 domain) comprises the following amino acid substitutions (i.e. destabilizing mutations), wherein the numbering is according to the Kabat numbering system:
either the CH3 domain with the hole mutation comprises at least one amino acid substitution, i.e. destabilizing mutation, selected from the group of replacement of S354 with a hydrophobic amino acid; replacement of D356 with a positively charged amino acid; replacement of E357 with a positively charged amino acid or with a hydrophobic amino acid; replacement of D356 with a positively charged amino acid, and replacement of E357 with a positively charged amino acid or with a hydrophobic amino acid; replacement of S364 with a hydrophobic amino acid; replacement of A368 with a hydrophobic amino acid; replacement of E392 with a negatively charged amino acid; replacement of T394 with a hydrophobic amino acid; replacement of D399 with a hydrophobic amino acid and replacement of S400 with a positively charged amino acid; replacement of D399 with a hydrophobic amino acid and replacement of F405 with a positively charged amino acid; replacement of V407 with a hydrophobic amino acid; and replacement of K409 with a negatively charged amino acid; and replacement of K439 with a negatively charged amino acid; or
the CH3 domain with the knob mutation comprises at least one amino acid substitution, i.e. destabilizing mutation, selected from the group of replacement of Q347 with a positively charged amino acid, and replacement of K360 with a negatively charged amino acid; replacement of Y349 with a negatively charged amino acid; replacement of L351 with a hydrophobic amino acid, and replacement of E357 with a hydrophobic amino acid; replacement of S364 with a hydrophobic amino acid; replacement of W366 with a hydrophobic amino acid, and replacement of K409 with a negatively charged amino acid; replacement of L368 with a hydrophobic amino acid; replacement of K370 with a negatively charged amino acid; replacement of K370 with a negatively charged amino acid, and replacement of K439 with a negatively charged amino acid; replacement of K392 with a negatively charged amino acid; replacement of T394 with a hydrophobic amino acid; replacement of V397 with a hydrophobic amino acid; replacement of D399 with a positively charged amino acid, and replacement of K409 with a negatively charged amino acid; replacement of S400 with a positively charged amino acid; F405W; Y407W; and replacement of K439 with a negatively charged amino acid.

In another aspect the invention provides a second heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation, wherein the second heterodimeric precursor polypeptide comprises a second antigen binding moiety, wherein at least a part of the second antigen binding moiety is arranged on one of the two polypeptide chains comprising the CH3 domain; and wherein one of the CH3 domains (but not the other CH3 domain) comprises the following amino acid substitutions (i.e. destabilizing mutations), wherein the numbering is according to the Kabat numbering system:
either the CH3 domain with the hole mutation comprises at least one amino acid substitution, i.e. destabilizing mutation, selected from the group of replacement of S354 with a hydrophobic amino acid; replacement of D356 with a positively charged amino acid; replacement of E357 with a positively charged amino acid or with a hydrophobic amino acid; replacement of D356 with a positively charged amino acid, and replacement of E357 with a positively charged amino acid or with a hydrophobic amino acid; replacement of S364 with a hydrophobic amino acid; replacement of A368 with a hydrophobic amino acid; replacement of E392 with a negatively charged amino acid; replacement of T394 with a hydrophobic amino acid; replacement of D399 with a hydrophobic amino acid and replacement of S400 with a positively charged amino acid; replacement of D399 with a hydrophobic amino acid and replacement of F405 with a positively charged amino acid; replacement of V407 with a hydrophobic amino acid; and replacement of K409 with a negatively charged amino acid; and replacement of K439 with a negatively charged amino acid; or
the CH3 domain with the knob mutation comprises at least one amino acid substitution, i.e. destabilizing mutation, selected from the group of replacement of Q347 with a positively charged amino acid, and replacement of K360 with a negatively charged amino acid; replacement of Y349 with a negatively charged amino acid; replacement of L351 with a hydrophobic amino acid, and replacement of E357 with a hydrophobic amino acid; replacement of S364 with a hydrophobic amino acid; replacement of W366 with a hydrophobic amino acid, and replacement of K409 with a negatively charged amino acid; replacement of L368 with a hydrophobic amino acid; replacement of K370 with a negatively charged amino acid; replacement of K370 with a negatively charged amino acid, and replacement of K439 with a negatively charged amino acid; replacement of K392 with a negatively charged amino acid; replacement of T394 with a hydrophobic amino acid; replacement of V397 with a hydrophobic amino acid; replacement of D399 with a positively charged amino acid, and replacement of K409 with a negatively charged amino acid; replacement of S400 with a positively charged amino acid; F405W; Y407W; and replacement of K439 with a negatively charged amino acid.

In yet another aspect the invention provides the use of a first heterodimeric precursor polypeptide according to the invention in combination with a second heterodimeric polypeptide according to the invention for the formation of a heterodimeric product polypeptide. In one embodiment the first heterodimeric precursor polypeptide according to the invention is used in combination with a second heterodimeric polypeptide according to the invention for the formation of a heterodimeric product polypeptide by polypeptide chain exchange.

In yet another aspect the invention provides the use of a second heterodimeric precursor polypeptide according to the invention in combination with a first heterodimeric polypeptide according to the invention for the formation of a heterodimeric product polypeptide. In one embodiment the second heterodimeric precursor polypeptide according to the invention is used in combination with a first heterodimeric polypeptide according to the invention for the formation of a heterodimeric product polypeptide by polypeptide chain exchange.

In another aspect of the invention the use of a first heterodimeric precursor polypeptide according to the invention in a set of heterodimeric precursor polypeptides according to the invention is provided. In another aspect of the invention the use of a second heterodimeric precursor polypeptide according to the invention in a set of heterodimeric precursor polypeptides according to the invention is provided.

Another aspect of the invention is the use of a first heterodimeric precursor polypeptide according to the invention in a method for generating a heterodimeric polypeptide according to the invention. Another aspect of the invention is the use of a second heterodimeric precursor polypeptide according to the invention in a method for generating a heterodimeric polypeptide according to the invention.

Another aspect of the invention is the use of a first heterodimeric precursor polypeptide according to the invention in a method for identifying a multispecific heterodimeric polypeptide according to the invention. Another aspect of the invention is the use of a second heterodimeric precursor polypeptide according to the invention in a method for identifying a multispecific heterodimeric polypeptide according to the invention.

Another aspect of the invention is the use of the set of heterodimeric precursor polypeptides according to the invention in a method for generating a heterodimeric polypeptide according to the invention. Another aspect of the invention is the use of set of heterodimeric precursor polypeptides according to the invention in a method for identifying a multispecific heterodimeric polypeptide according to the invention.

In one embodiment, the following applied to the first and the second heterodimeric precursor polypeptide:
in case the CH3 domain with the knob mutation comprises a mutation E357K, the CH3 domain with the hole mutation does not comprise a mutation K370E; and
in case the CH3 domain with the knob mutation comprises a mutation D356K, the CH3 domain with the hole mutation does not comprise a mutation K439E.

In other words, the following applies according to one embodiment of the invention for precursor polypeptides as provided with the invention:
in case the CH3 domain with the knob mutation comprises an amino acid substitution E357K, the CH3 domain with the hole mutation comprises K at position 370; and
in case the CH3 domain with the knob mutation comprises an amino acid substitution D356K, the CH3 domain with the hole mutation comprises K at position 439.

In one embodiment the first heterodimeric precursor polypeptide comprises at least two (in one embodiment exactly two) polypeptide chains comprising a CH3 domain, wherein one of the two polypeptide chains comprising the CH3 domain comprises at least a part of a (first) antigen binding moiety specifically binding to an antigen; and wherein the other one of the two polypeptide chains comprising the CH3 domain does not comprise an antigen binding moiety specifically binding to an antigen. In one embodiment the second heterodimeric precursor polypeptide comprises at least two (in one embodiment exactly two) polypeptide chains comprising a CH3 domain, wherein one of the two polypeptide chains comprising the CH3 domain comprises at least a part of a (first) antigen binding moiety specifically binding to an antigen; and wherein the other one of the two polypeptide chains comprising the CH3 domain does not comprise an antigen binding moiety specifically binding to an antigen. In one embodiment the first heterodimeric precursor polypeptide comprises at least two (in one embodiment exactly two) polypeptide chains comprising a CH3 domain, wherein one of the two polypeptide chains comprising the CH3 domain comprises at least a part of a (first) antigen binding moiety specifically binding to an antigen; and wherein the other one of the two polypeptide chains comprising the CH3 domain does not comprise an antigen binding moiety specifically binding to an antigen; and the second heterodimeric precursor polypeptide comprises at least two (in one embodiment exactly two) polypeptide chains comprising a CH3 domain, wherein one of the two polypeptide chains comprising the CH3 domain comprises at least a part of a (first) antigen binding moiety specifically binding to an antigen; and wherein the other one of the two polypeptide chains comprising the CH3 domain does not comprise an antigen binding moiety specifically binding to an antigen. In other words, according to this embodiment of the invention one or more functional antigen binding moieties are arranged on only one of the two polypeptide chains comprising the CH3 domain, while on the other polypeptide chain comprising the CH3 domain no functional antigen binding moiety is arranged. This polypeptide chain is herein also referred to as “dummy polypeptide”. In one embodiment the dummy polypeptide is only associated with the other polypeptide chain comprising the CH3 domain, i.e. in the heterodimer, but is not associated with another (e.g. a third) polypeptide chain. The dummy polypeptide may comprise parts of antigen binding moieties, e.g. an antibody variable domain, which are not involved in a functional antigen binding site within the heterodimeric precursor polypeptide. One advantage of such arrangement, e.g. combination of a dummy polypeptide comprising a CH3 domain with a polypeptide chain comprising a CH3 domain that is involved in formation of one or more functional antigen binding sites, the product polypeptide formed upon polypeptide chain exchange is of different size than the heterodimeric precursor molecules, which allows for improved of product polypeptide(s) from unreacted precursor polypeptides.

As indicated, in each one of the heterodimeric precursor polypeptide one of the polypeptide chains comprising the CH3 domain comprises a CH3 domain with a knob mutation and the other polypeptide chain comprising the CH3 domain comprises a CH3 domain with a hole mutation. Upon polypeptide chain exchange the polypeptide chain comprising the CH3 domain with the knob mutation from the first precursor polypeptide forms a heterodimer (i.e. a first heterodimeric product polypeptide) with the polypeptide chain comprising the CH3 domain with the hole from the second precursor polypeptide, and the polypeptide chain comprising the CH3 domain with the hole mutation from the first precursor polypeptide forms a heterodimer (i.e. a second heterodimeric product polypeptide) with the polypeptide chain comprising the CH3 domain with the knob from the second precursor polypeptide.

As indicated, one CH3 domain comprised in the first heterodimeric precursor polypeptide comprises one or more destabilizing mutations, as indicated above, while the other CH3 domain comprised in said first heterodimeric precursor polypeptide does not comprise a destabilizing mutation; and one CH3 domain comprised in the second heterodimeric polypeptide comprises one or more destabilizing mutations, as indicated above, while the other CH3 domain comprised in said second heterodimeric precursor polypeptide does not comprise a destabilizing mutation. The destabilizing mutations present in the precursor polypeptides are arranged such that they are present in the same product polypeptide after polypeptide chain exchange. Hence, in one of the precursor polypeptide the one or more destabilizing mutations are arranged in the CH3 domain comprising the knob mutation and in the other precursor polypeptide the one or more destabilizing mutations are arranged in the CH3 domain comprising the hole mutation.

In one embodiment within the first heterodimeric precursor polypeptide the polypeptide chain comprising the CH3 domain comprising the knob mutation comprises at least a part of the first antigen binding moiety and within the second heterodimeric precursor polypeptide the polypeptide chain comprising the CH3 domain with the hole mutation comprises at least a part of the second antigen binding moiety.

In one embodiment within the first heterodimeric precursor polypeptide the polypeptide chain comprising the CH3 domain comprising the hole mutation comprises at least a part of the first antigen binding moiety and within the second heterodimeric precursor polypeptide the polypeptide chain comprising the CH3 domain with the knob mutation comprises at least a part of the second antigen binding moiety.

In one embodiment a heterodimeric precursor polypeptide comprises exactly two polypeptide chains comprising a CH3 domain.

In one embodiment the CH3 domain comprising a destabilizing mutation comprises one, two or three destabilizing mutations. In one embodiment the CH3 domain comprising a destabilizing mutation comprises one or two destabilizing mutations.

The invention includes heterodimeric precursor polypeptides, wherein in the CH3 domain having a knob mutation, the knob mutation is replaced by a destabilizing mutation. For example, a destabilizing mutation may be arranged at position 366 in the CH3 domain with the knob mutation, which is e.g. T366W. Thus, this heterodimeric precursor polypeptide comprises the destabilizing mutation, i.e. a hydrophobic amino acid, at position 366 in the CH3 domain but not tryptophan (W). Yet, a heterodimeric precursor polypeptide having such substitution is considered to be encompassed by the invention.

Also, the invention includes heterodimeric precursor polypeptides, wherein in the CH3 domain having a hole mutation, one or more of the mutation(s) is replaced by a destabilizing mutation. For example, a destabilizing mutation may be arranged at position 368 in the CH3 domain with the hole mutation, which is e.g. T366S L368A Y407V. Thus, this heterodimeric precursor polypeptide comprises the destabilizing mutation, i.e. another hydrophobic amino acid, at position 368 in the CH3 domain but not alanine (A). In another example, a destabilizing mutation may be arranged at position 407 in the CH3 domain with the hole mutation, which is e.g. T366S L368A Y407V. Thus, this heterodimeric precursor polypeptide comprises the destabilizing mutation, i.e. another hydrophobic amino acid, at position 407 in the CH3 domain but not valine (V). Yet, such heterodimeric precursor polypeptides having such substitution or substitutions are considered to be encompassed by the invention.

In one embodiment of the invention no interchain disulfide bond is formed between the two polypeptide chains comprising the CH3 domains of the first heterodimeric polypeptide. In one embodiment of the invention no interchain disulfide bond is formed between the two polypeptide chains comprising the CH3 domains of the second heterodimeric polypeptide. In one embodiment of the invention no interchain disulfide bond is formed between the two polypeptide chains comprising the CH3 domains of the first heterodimeric polypeptide and the second heterodimeric polypeptide. Heterodimeric precursor polypeptides devoid of interchain disulfide bonds between the two polypeptide chains comprising the CH3 domains are capable of undergoing a polypeptide chain exchange in absence of a reducing agent. Hence, heterodimeric precursor polypeptides, wherein between the polypeptide chains comprising the CH3 domains no interchain disulfide bonds are present, are particularly suitable for applications in which the presence of reducing agents is not possible or not desired; e.g. for application in therapy.

### A) Amino Acid Substitutions in CH3 Domains

Precursor polypeptides, as provided by the invention, comprise amino acid substitutions in their CH3 domains.

### Knobs-into-Holes Mutations

In one embodiment the knob mutation comprised in the first heterodimeric precursor polypeptide is identical to the knob mutation comprised in the second heterodimeric precursor polypeptide.

In one embodiment the knob mutation is T366W. In one embodiment the hole mutation is T366S L368A Y407V.

### Destabilizing Mutations

As indicated above, only one CH3 domain of each precursor polypeptide comprises one or more destabilizing mutations.

According to the invention, either i) the CH3 domain of the first heterodimeric precursor polypeptide comprising the knob mutation and the CH3 domain of the second heterodimeric precursor polypeptide comprising the hole mutation, or ii) the CH3 domain of the first heterodimeric precursor polypeptide comprising the hole mutation and the CH3 domain of the second heterodimeric precursor polypeptide comprising the knob mutation comprise one or more destabilizing mutations. The one or more destabilizing mutations within the first and second heterodimeric precursor polypeptide are selected such that they interact in the CH3/CH3 interface of the product polypeptide formed by polypeptide chain exchange between the precursor polypeptides.

In case the CH3 domain comprising a knob mutation of a heterodimeric precursor polypeptide comprises a destabilizing mutation, the CH3 domain comprising the hole mutation of said heterodimeric precursor polypeptide does not comprise a destabilizing mutation. When a CH3 domain “does not comprise a destabilizing mutation” it comprises the wild type amino acid residue at the position interacting in a wild type immunoglobulin CH3 domain of the same class with the amino acid residue at the position of the destabilizing mutation comprised in the corresponding CH3 domain.

In one embodiment of the invention, the CH3 domain with the hole mutation comprises at least one amino acid substitution, i.e. destabilizing mutation, selected from the group of replacement of S354 with a hydrophobic amino acid; replacement of D356 with a positively charged amino acid; replacement of E357 with a positively charged amino acid or with a hydrophobic amino acid; replacement of D356 with a positively charged amino acid, and replacement of E357 with a positively charged amino acid or with a hydrophobic amino acid; replacement of S364 with a hydrophobic amino acid; replacement of A368 with a hydrophobic amino acid; replacement of E392 with a negatively charged amino acid; replacement of T394 with a hydrophobic amino acid; replacement of D399 with a hydrophobic amino acid and replacement of S400 with a positively charged amino acid; replacement of D399 with a hydrophobic amino acid and replacement of F405 with a positively charged amino acid; replacement of V407 with a hydrophobic amino acid; and replacement of K409 with a negatively charged amino acid; and replacement of K439 with a negatively charged amino acid; and the CH3 domain with the knob mutation comprises at least one amino acid substitution, i.e. destabilizing mutation, selected from the group of replacement of Q347 with a positively charged amino acid, and replacement of K360 with a negatively charged amino acid; replacement of Y349 with a negatively charged amino acid; replacement of L351 with a hydrophobic amino acid, and replacement of E357 with a hydrophobic amino acid; replacement of S364 with a hydrophobic amino acid; replacement of W366 with a hydrophobic amino acid, and replacement of K409 with a negatively charged amino acid; replacement of L368 with a hydrophobic amino acid; replacement of K370 with a negatively charged amino acid; replacement of K370 with a negatively charged amino acid, and replacement of K439 with a negatively charged amino acid; replacement of K392 with a negatively charged amino acid; replacement of T394 with a hydrophobic amino acid; replacement of V397 with a hydrophobic amino acid; replacement of D399 with a positively charged amino acid, and replacement of K409 with a negatively charged amino acid; replacement of S400 with a positively charged amino acid; F405W; Y407W; and replacement of K439 with a negatively charged amino acid.

In one embodiment the hydrophobic amino acid is selected from Norleucine, Met, Ala, Val, Leu, Ile, Trp, Tyr, and Phe. In one embodiment the hydrophobic amino acid is selected from Ala, Val, Leu, Ile and Tyr. In one embodiment the hydrophobic amino acid is Val, Leu, or Ile. In one embodiment the hydrophobic amino acid is Leu or Ile. In one embodiment the hydrophobic amino acid is Leu. In one embodiment the hydrophobic amino acid is Tyr. In one embodiment the hydrophobic amino acid is Phe.

In one embodiment the positively charged amino acid is His, Lys, or Arg. In one embodiment the positively charged amino acid is Lys, or Arg. In one embodiment the positively charged amino acid is Lys.

In one embodiment the negatively charged amino acid is Asp or Glu. In one embodiment the negatively charged amino acid is Asp. In one embodiment the negatively charged amino acid is Glu.

Amino acid substitutions with amino acids having the respective side-chain properties at the indicated amino acid positions in the CH3 domain were found to support polypeptide chain exchange and product polypeptide formation from two precursor polypeptides.

In one embodiment of the invention, the CH3 domain with the hole mutation comprises at least one amino acid substitution selected from the group of S354V, S354I, S354L, D356K, D356R, E357K, E357R, E357F, S364L, S364I, A368F, K392D, K392E, T394L, T394I, V407Y, K409E, K409D, K439D, K439E and a double mutation D399A S400K, D399A S400R, D399A F405W; and the CH3 domain with the knob mutation comprises at least one amino acid substitution selected from the group of Y349E, Y349D, S364V, S364I, S364L, L368F, K370E, K370D, K392E, K392D, T394L, T394I, V397Y, S400K, S400R, F405W, Y407W, K349E, K439D and double mutations Q347K K360E, Q347R K360E, Q347K K360D, Q347R K360D, L351F E357F, W366I K409E, W366L K409E, W366K K409D, W366L K409D, D399K K409E, D399R K409E, D399K K409D, and D399K K409E.

In one embodiment of the invention, the CH3 domain with the hole mutation comprises at least one amino acid substitution selected from the group of S354V, D356K, E357K, E357F, S364L, A368F, K392E, T394I, V407Y, K409E, K439E and a double mutation D399A S400K; and the CH3 domain with the knob mutation comprises at least one amino acid substitution selected from the group of Y349E, S364V, L368F, K370E, K392D, T394I, V397Y, S400K, F405W, Y407W, K349E, and double mutations Q347K K360E, L351F E357F, W366I K409E, and D399K K409E.

In one embodiment of the invention, the CH3 domain with the hole mutation comprises at least one amino acid substitution selected from the group of D356K, D356R, E357K, E357R, E357F, S364L, S364I, V407Y, K409E, K409D and a double mutation D399A S400K, D399A S400R; and the CH3 domain with the knob mutation comprises at least one amino acid substitution selected from the group of Y349E, Y349D, K370E, K370D, K392E, K392D, T394L, T394I, V397Y, F405W, Y407W, K349E, K439D and double mutations Q347K K360E, Q347R K360E, Q347K K360D, Q347R K360D, W366I K409E, W366L K409E, W366K K409D, W366L K409D, D399K K409E, D399R K409E, D399K K409D, and D399K K409E.

In one embodiment of the invention, the CH3 domain with the hole mutation comprises at least one amino acid substitution selected from the group of D356K, E357K, E357F, S364L, V407Y, K409E, and a double mutation D399A S400K; and the CH3 domain with the knob mutation comprises at least one amino acid substitution selected from the group of Y349E, K370E, K392, T394I, V397Y, F405W, Y407W, K349E, and double mutations Q347K K360E, W366I K409E, and D399K K409E.

In one embodiment of the invention, the CH3 domain with the hole mutation and the CH3 domain with the knob mutation that comprise the destabilizing mutations comprise one of the amino acid substitutions selected from the group indicated in the following table:

| CH3 domain comprising | CH3 domain comprising |
|---|---|
| hole mutation | knob mutation |
| D356K | K370E K439E |
| D356K E357K | Y349E |
| D356K E357K | W366I K409E |
| D356K E357K | K370E K439E |
| D356K E357K | K392D |
| D356K E357K | T394I |
| D356K E357K | D399K K409E |
| D356K E357K | F405W |
| D356K E357K | Y407W |
| E357K | Y349E |
| E357K | W366I K409E |
| E357K | K370E K439E |
| E357K | T394I |
| E357K | D399K K409E |
| E357K | F405W |
| E357K | Y407W |
| E357F | Q347K K360E |
| E357F | Y349E |
| E357F | W366I K409E |
| E357F | K370E K439E |
| E357F | T394I |
| E357F | D399K K409E |
| E357F | Y407W |
| S364L | Y349E |
| S364L | K392D |
| S364L | F405W |
| S364L | W366I K409E |
| S364L | K370E K439E |
| S364L | T394I |
| S364L | D399K K409E |
| S364L | Y407W |
| A368F | T394I |
| D399A S400K | W366I K409E |
| D399A S400K | T394I |
| V407Y | Q347K K360E |
| V407Y | Y349E |
| V407Y | W366I K409E |
| V407Y | T394I |
| V407Y | D399K K409E |
| V407Y | F405W |
| V407Y | Y407W |
| V407Y | K392D |
| K409E | Y349E |
| K409E | W366I K409E |
| K409E | K392D |
| K409E | T394I |
| K409E | D399K K409E |
| K409E | F405W |
| K409E | Y407W |

For clarity, this table is to be understood in that the CH3 domain comprising the hole mutation comprises a destabilizing mutation as indicated in the first column of above table, the CH3 domain comprising the knob mutation comprises the destabilizing mutation listed in the right column of above table, indicated in the same line.

In one embodiment of the invention, the CH3 domain with the hole mutation and the CH3 domain with the knob mutation that comprise the destabilizing mutations comprise one of the amino acid substitutions selected from the group indicated in the following table:

| CH3 domain comprising | CH3 domain comprising |
|---|---|
| hole mutation | knob mutation |
| V407Y | K370E |
| V407Y | K370E K439E |
| V407Y | V397Y |
| S364L | K439E |
| S364L | W366I K409E |
| S364L | K370E K439E |
| S364L | D399K K409E |
| S364L | T394I |
| S364L | W366I K409E |
| S364L | K370E K439E |
| S364L | D399K K409E |
| S364L | T394I |

In one embodiment of the invention, the CH3 domain with the hole mutation and the CH3 domain with the knob mutation that comprise the destabilizing mutations comprise one of the amino acid substitutions selected from the group indicated in the following table:

| CH3 domain comprising | CH3 domain comprising |
|---|---|
| hole mutation | knob mutation |
| T394I E357F | Y349E Y407W |
| S364L E357F | Y349E Y407W |
| E357K T394I | Y349E K439E |
| E357K T394I | Y349E W366I K409E |
| D356K V407Y | W366I K409E F405W |
| S364L E357F | W366I K409E K439E |
| T394I E357F | W366I K409E K439E |
| S364L D356K | W366I K409E K439E |
| D356K V407Y | T394I F405W |
| S364L D356K | T394I F405W |

In one embodiment of the invention, the CH3 domain with the hole mutation and the CH3 domain with the knob mutation that comprise the destabilizing mutations comprise one of the amino acid substitutions selected from the group indicated in the following table:

| CH3 domain comprising | CH3 domain comprising |
|---|---|
| hole mutation | knob mutation |
| E357F S364L | K370E K439E |
| E357F S364L | Y349E W366I K409D |
| E357F S364L | Y349E K439E |
| D356K V407Y | K370E K439E |
| E357F S364L | Y407W |
| D356K V407Y | Y349E W366I K409D |
| E357F S364L | Y349E Y407W |
| D356K V407Y | Y349E K439E |
| E357F S364L | W366I F405W K409D |
| E357F S364L | D399K K409E |
| D356K V407Y | W366I K409D K439E |
| S364I | Y349E K439E |
| D356K V407Y | D399K K409E |
| D356K V407Y | W366I K409D |
| D356K V407Y | T394I F405W |
| V407Y | D399K K409E |
| E357K T394I | D399K K409E |
| E357K T394I | W366I K409D |

For clarity, this table is to be understood in that the CH3 domain comprising the hole mutation comprises a destabilizing mutation as indicated in the first column of above table, the CH3 domain comprising the knob mutation comprises the destabilizing mutation listed in the right column of above table, indicated in the same line. Precursor molecules with this combination of destabilizing mutations exhibit particular beneficial polypeptide chain exchange.

In one embodiment of the invention, the CH3 domain with the hole mutation and the CH3 domain with the knob mutation that comprise the destabilizing mutations comprise one of the amino acid substitutions selected from the group indicated in the following table:

| CH3 domain comprising | CH3 domain comprising |
|---|---|
| hole mutation | knob mutation |
| E357F S364L | Y349D |
| S364A | Y349D |
| E357F S364L | Y407W |
| E357F S364L | Y349E |
| S364A | Y349E |
| E357F S364L | W336I F405W K409D |
| S364A | W336I F405W K409D |
| E357F S364L | D399K K409E |
| S364A | D399K K409E |
| S364I | Y349D |
| S364I | Y407W |
| S364I | Y349E |
| S364I | W336I F405W K409D |
| S364L | Y349D |
| S364I | D399K K409E |
| S364L | Y407W |
| K409E | Y349D |
| S364L | Y349E |
| S364L | W336I F405W K409D |
| K409E | Y407W |
| K409E | Y349E |
| S364L | D399K K409E |
| K409E | W336I F405W K409D |
| K409E | D399K K409E |

For clarity, this table is to be understood in that the CH3 domain comprising the hole mutation comprises a destabilizing mutation as indicated in the first column of above table, the CH3 domain comprising the knob mutation comprises the destabilizing mutation listed in the right column of above table, indicated in the same line. Precursor molecules with this combination of destabilizing mutations exhibit particular beneficial polypeptide chain exchange while being producible in high yields.

### Cysteine Mutations

In one embodiment of the invention, the CH3 domains of the heterodimeric precursor polypeptides comprise a third pattern of mutations, i.e. substitutions of distinct amino acids in the CH3/CH3 interface by cysteine in order to allow formation of interchain disulfide bonds between two CH3 domains having cysteine substitutions at interacting positions.

Thus, in one embodiment of the invention either i) the CH3 domain comprising the knob mutation of the first heterodimeric precursor polypeptide comprises a cysteine mutation and the CH3 domain comprising the hole mutation of the second heterodimeric precursor polypeptide comprises a cysteine mutation, or ii) the CH3 domain comprising the hole mutation of the first heterodimeric precursor polypeptide comprises a cysteine mutation and the CH3 domain comprising the knob mutation of the second heterodimeric precursor polypeptide comprises a cysteine mutation. In other words in one embodiment, either i) within the first heterodimeric polypeptide the CH3 domain comprising the knob mutation comprises a cysteine mutation and the CH3 domain comprising the hole mutation does not comprise a cysteine mutation and within the second heterodimeric polypeptide the CH3 domain comprising the knob mutation does not comprise a cysteine mutation and the CH3 domain comprising the hole mutation comprises a cysteine mutation, or ii) within the first heterodimeric polypeptide the CH3 domain comprising the knob mutation does not comprise a cysteine mutation and the CH3 domain comprising the hole mutation comprises a cysteine mutation and within the second heterodimeric polypeptide the CH3 domain comprising the knob mutation comprises a cysteine mutation and the CH3 domain comprising the hole mutation does not comprise a cysteine mutation.

In one embodiment, either i) the CH3 domain comprising the knob mutation of the first heterodimeric precursor polypeptide comprises a first cysteine mutation and the CH3 domain comprising the hole mutation of the second heterodimeric precursor polypeptide comprises a second cysteine mutation, or ii) the CH3 domain comprising the hole mutation of the first heterodimeric precursor polypeptide comprises a first cysteine mutation and the CH3 domain comprising the knob mutation of the second heterodimeric precursor polypeptide comprises a second cysteine mutation, wherein the first and second cysteine mutations are selected from the following pairs:

| First cysteine mutation | Second cysteine mutation |
|---|---|
| D399C | K392C |
| Y349C | S354C |
| Y349C | E356C |
| Y349C | E357C |
| L351C | S354C |
| T394C | V397C |

In one embodiment the first cysteine mutation is Y349C and the second cysteine mutation is S354C.

In one embodiment of the invention i) the CH3 domain comprising the knob mutation of the first heterodimeric precursor polypeptide comprises a substitution S354C and the CH3 domain comprising the hole mutation of the second heterodimeric precursor polypeptide comprises a substitution Y349C, or ii) the CH3 domain comprising the hole mutation of the first heterodimeric precursor polypeptide comprises a substitution Y349C and the CH3 domain comprising the knob mutation of the second heterodimeric precursor polypeptide comprises a substitution S354C.

In one embodiment of the invention, within the first heterodimeric precursor polypeptide the CH3 domain comprising the knob mutation comprises a substitution S354C and the CH3 domain comprising the hole mutation comprises Y at position 349; and wherein within the second heterodimeric precursor polypeptide the CH3 domain comprising the hole mutation comprises a substitution Y349C and the CH3 domain comprising the knob mutation comprises S at position 354.

In one embodiment of the invention i) the CH3 domain comprising the knob mutation of the first heterodimeric precursor polypeptide comprises substitutions T366W S354C and the CH3 domain comprising the hole mutation of the second heterodimeric precursor polypeptide comprises substitutions T366S L368A Y407V Y349C, or ii) the CH3 domain comprising the hole mutation of the first heterodimeric precursor polypeptide comprises substitutions T366S L368A Y407V Y349C and the CH3 domain comprising the knob mutation of the second heterodimeric precursor polypeptide comprises substitutions T366W S354C.

In one embodiment of the invention, within the first heterodimeric precursor polypeptide the CH3 domain comprising the knob mutation comprises a substitution T366W S354C and the CH3 domain comprising the hole mutation comprises Y at position 349 and substitutions T366S L368A Y407V; and wherein within the second heterodimeric precursor polypeptide the CH3 domain comprising the hole mutation comprises substitutions T366S L368A Y407V Y349C and the CH3 domain comprising the knob mutation comprises S at position 354 and a substitution T366W.

In one embodiment of the invention, the CH3 domains of the heterodimeric precursor polypeptides do not comprise an interchain disulfide bond.

### B) Antigen Binding Moiety

In one embodiment of the invention, the antigen binding moiety is a polypeptide specifically binding to an antigen. In one embodiment the antigen binding moiety is selected from the group of antibodies, receptors, ligands, and DARPins capable of specifically binding to an antigen.

In one embodiment of the invention the antigen binding moiety comprised in a (precursor) polypeptide according to the invention is an antibody fragment.

In one embodiment of the invention the antigen binding moiety comprises a pair of a VH domain and a VL domain, which form an antigen binding site specifically binding to a target antigen.

In one embodiment of the invention the antibody fragment comprised in a (precursor) polypeptide according to the invention is an antibody fragment selected from the group of Fv, Fab, Fab′, Fab′-SH, F(ab′)₂, diabodies, scFv, and scFab. In one embodiment the antibody fragment comprised in a (precursor) polypeptide according to the invention is a Fv or a Fab.

In one embodiment of the invention, the antigen binding moiety is a Fab fragment.

In one embodiment of the invention, the first antigen binding moiety is a first Fab fragment and the second antigen binding moiety is a second Fab fragment. In one embodiment of the invention, the first Fab fragment, the second Fab fragment or both, the first and the second Fab fragment are altered by a domain crossover, such that either:
a) only the CH1 and CL domains are replaced by each other;
b) only the VH and VL domains are replaced by each other; or
c) the CH1 and CL domains are replaced by each other and the VH and VL domains are replaced by each other.

In one embodiment of the invention, the antigen binding moiety is a Fv fragment. In one embodiment of the invention, the first antigen binding moiety is a first Fv fragment and the second antigen binding moiety is a second Fv fragment.

In one embodiment of the invention, the antigen binding moiety of the first heterodimeric precursor polypeptide and the antigen binding moiety of the second heterodimeric precursor polypeptide bind to the same antigen. In one embodiment of the invention, the antigen binding moiety of the first heterodimeric precursor polypeptide and the antigen binding moiety of the second heterodimeric precursor polypeptide are identical antigen binding moieties.

In one embodiment of the invention, the antigen binding moiety of the first heterodimeric precursor polypeptide and the antigen binding moiety of the second heterodimeric precursor polypeptide bind to different antigens. In this case, upon polypeptide chain exchange between two heterodimeric precursor polypeptides, a multispecific product polypeptide is formed, which comprises the antigen binding moiety originating from the first heterodimeric precursor polypeptide and the antigen binding moiety originating from the second heterodimeric precursor polypeptide.

Further antigen binding moieties may be present in the heterodimeric precursor polypeptide, which may be fused to the N-terminus or the C-terminus of a polypeptide chain comprised in the heterodimeric precursor polypeptide in order to provide product polypeptide of higher valence.

Such further antigen binding moieties are fused to the polypeptide chain via an appropriate peptide connector. In one embodiment the peptide connector is a glycine serine linker.

In one embodiment of the invention in a heterodimeric precursor polypeptide only one of the polypeptide chains comprising a CH3 domain of comprises at least a part of an antigen binding moiety. In one embodiment of the invention in a heterodimeric precursor polypeptide one of the polypeptide chains comprising a CH3 domain of an antigen binding site specifically binding to a target antigen. In one embodiment of the invention in a heterodimeric precursor polypeptide one of the polypeptide chains comprising the CH3 domain comprises from N- to C-terminal direction a hinge region, an antibody variable domain and a CH3 domain, and the polypeptide chain is not part of an antigen binding site specifically binding to a target antigen. In one embodiment of the invention in a heterodimeric precursor polypeptide one of the polypeptide chains comprising the CH3 domain comprises from N- to C-terminal direction a hinge region, an antibody variable domain, a CH2 domain and a CH3 domain, and the polypeptide chain is not part of an antigen binding site specifically binding to a target antigen.

### C) Domain Arrangement of Precursor Polypeptides

Precursor polypeptides according to the invention are suitable for the generation of product polypeptides of various formats and with various domain arrangements. Depending on the selection of domains and the number of antigen binding moieties provided in the heterodimeric precursor molecules, product polypeptides with different antigen binding characteristics (e.g. specificity, valency) and different effector functions may be generated.

In one embodiment the first heterodimeric precursor polypeptide and the second heterodimeric precursor polypeptide comprise exactly two polypeptide chains comprising a CH3 domain. Thus, further polypeptide chains devoid of CH3 domains may be comprised in the first and second heterodimeric precursor polypeptide.

### Precursor Polypeptides Comprising Antibody Fragment

In one embodiment of the invention the antigen binding moiety comprises a pair of a VH domain and a VL domain, which form an antigen binding site specifically binding to a target antigen; and
a) the first heterodimeric precursor polypeptide comprises:
   a first heavy chain polypeptide comprising a CH3 domain and a first antibody variable domain,
   a second heavy chain polypeptide comprising a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
   a light chain polypeptide comprising a second antibody variable domain, wherein the first and second antibody variable domain together form a first antigen binding site specifically binding to a target antigen; and wherein
b) the second heterodimeric precursor polypeptide comprises:
   a third heavy chain polypeptide comprising a CH3 domain and a third antibody variable domain,
   a fourth heavy chain polypeptide comprising a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
   a light chain polypeptide comprising a fourth antibody variable domain, wherein the third and fourth antibody variable domain together form a second antigen binding site specifically binding to a target antigen; and wherein
c) either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation.

In one embodiment of the invention the antigen binding moiety comprises a pair of a VH domain and a VL domain, which form an antigen binding site specifically binding to a target antigen; and
a) the first heterodimeric precursor polypeptide comprises:
   a first heavy chain polypeptide comprising from N- to C-terminal direction a first VH domain, and constant antibody domains CH1, CH2 and CH3,
   a second heavy chain polypeptide comprising from N- to C-terminal direction constant antibody domains CH2 and CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
   a light chain polypeptide comprising from N- to C-terminal direction a first VL domain and a CL domain, wherein the first VH domain and the first VL domain are associated with each other and form an antigen binding site specifically binding to a target antigen; and wherein
b) the second heterodimeric precursor polypeptide comprises:
   a third heavy chain polypeptide comprising from N- to C-terminal direction a second VH domain, and constant antibody domains CH1, CH2 and CH3,
   a fourth heavy chain polypeptide comprising from N- to C-terminal direction constant antibody domains CH2 and CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
   a light chain polypeptide comprising from N- to C-terminal direction a second VL domain and a CL domain, wherein the second VH domain and the second VL domain are associated with each other and form an antigen binding site specifically binding to a target antigen; and wherein
      either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation.

In one embodiment of the invention the antigen binding moiety comprises a pair of a VH domain and a VL domain, which form an antigen binding site specifically binding to a target antigen; and
a) the first heterodimeric precursor polypeptide comprises:
   a first heavy chain polypeptide comprising from N- to C-terminal direction a CH2 domain, a CH3 domain, a peptide connector, a first VH domain and a CH1 domain,
   a second heavy chain polypeptide comprising from N- to C-terminal direction constant antibody domains CH2 and CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
   a light chain polypeptide comprising from N- to C-terminal direction a first VL domain and a CL domain, wherein the first VH domain and the first VL domain are associated with each other and form an antigen binding site specifically binding to a target antigen; and wherein
b) the second heterodimeric precursor polypeptide comprises:
   a third heavy chain polypeptide comprising from N- to C-terminal direction a CH2 domain, a CH3 domain, a peptide connector, a first VH domain and a CH1 domain,
   a fourth heavy chain polypeptide comprising from N- to C-terminal direction constant antibody domains CH2 and CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
   a light chain polypeptide comprising from N- to C-terminal direction a second VL domain and a CL domain, wherein the second VH domain and the second VL domain are associated with each other and form an antigen binding site specifically binding to a target antigen; and wherein
      either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation.

In one embodiment of the invention the antigen binding moiety comprises a pair of a VH domain and a VL domain, which form an antigen binding site specifically binding to a target antigen; and
a) the first heterodimeric precursor polypeptide comprises:
   a first heavy chain polypeptide comprising from N- to C-terminal direction a first VH domain, and constant antibody domains CH1, CH2, CH3, a peptide connector, a first VH domain, and a CH1 domain,
   a second heavy chain polypeptide comprising from N- to C-terminal direction constant antibody domains CH2 and CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
   a light chain polypeptide comprising from N- to C-terminal direction a first VL domain and a CL domain, wherein the first VH domain and the first VL domain are associated with each other and form an antigen binding site specifically binding to a target antigen; and wherein
b) the second heterodimeric precursor polypeptide comprises:
   a third heavy chain polypeptide comprising from N- to C-terminal direction a second VH domain, and constant antibody domains CH1, CH2 and CH3, a peptide connector, a first VH domain, and a CH1 domain,
   a fourth heavy chain polypeptide comprising from N- to C-terminal direction constant antibody domains CH2 and CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
   a light chain polypeptide comprising from N- to C-terminal direction a second VL domain and a CL domain, wherein the second VH domain and the second VL domain are associated with each other and form an antigen binding site specifically binding to a target antigen; and wherein
      either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation.

Precursor Polypeptides Comprising CH2 Domain

In one embodiment of the invention, the first heterodimeric precursor polypeptide and the second heterodimeric precursor polypeptide comprise at least two polypeptide chains comprising a CH2 domain and the CH3 domain. Heterodimeric precursor polypeptides comprising CH2 domains and CH3 domains exhibit advantageous properties, such as long half-life in the circulation and mediation of Fc mediated effector function.

In one embodiment of the invention, the first heterodimeric precursor polypeptide and the second heterodimeric precursor polypeptide comprise at least two polypeptide chains comprising from N- to C-terminal direction a CH2 domain and the CH3 domain.

In one embodiment of the invention, either i) the first heterodimeric precursor polypeptide comprises one polypeptide chain comprising a VL domain, a CH2 domain and the CH3 domain, and wherein the second heterodimeric precursor polypeptide comprises one polypeptide chain comprising a VH domain, a CH2 domain and the CH3 domain, wherein said VL domain and said VH domain specifically bind to an antigen when associated to a pair of a VH domain and a VL domain; or ii) the first heterodimeric precursor polypeptide comprises one polypeptide chain comprising a VH domain, a CH2 domain and the CH3 domain, and wherein the second heterodimeric precursor polypeptide comprises one polypeptide chain comprising a VL domain, a CH2 domain and the CH3 domain, wherein said VL domain and said VH domain specifically bind to an antigen when associated to a pair of a VH domain and a VL domain.

In one embodiment of the invention, the first heterodimeric precursor polypeptide and the second heterodimeric precursor polypeptide are devoid of a CH2 domain. Heterodimeric precursor polypeptides devoid of CH2 domains may exhibit advantageous properties, such as fast clearance from the circulation.

Precursor Polypeptides Comprising Activatable Antigen Binding Site

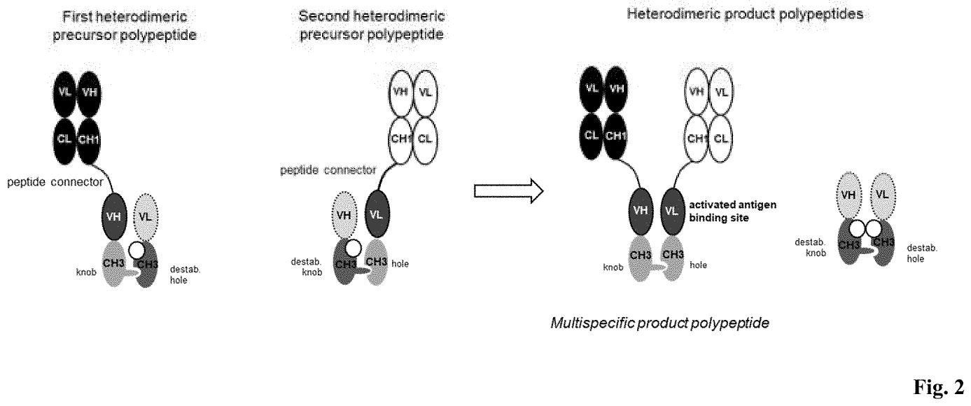

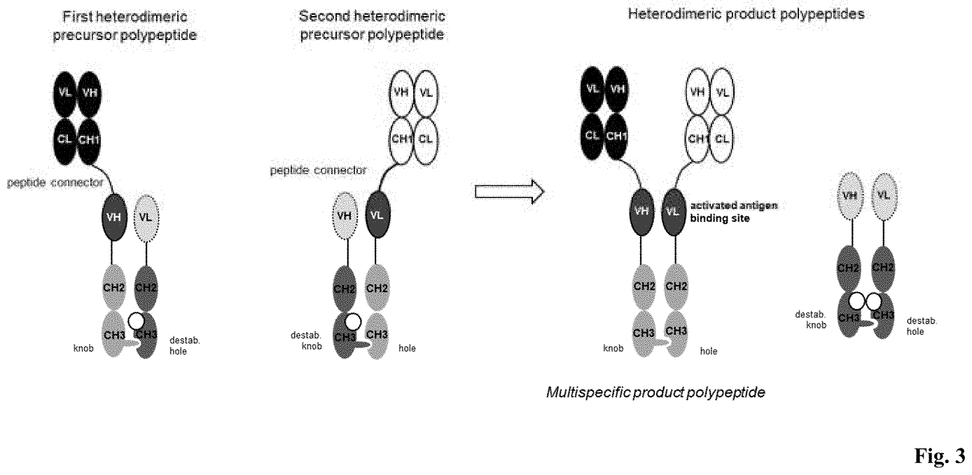

In one embodiment each precursor polypeptides comprises a part of an antigen binding moiety, wherein said antigen binding moiety is non-functional in the precursor polypeptide, and wherein in the product polypeptide formed by polypeptide chain exchange between the precursor polypeptides the antigen binding moiety is functional and specifically binds to a target antigen. An exemplary structure of such precursor polypeptides is indicated in FIG. 2 and FIG. 3.

In one embodiment of the invention said antigen binding moiety is an antigen binding site comprising a pair of antibody variable domains.

In one embodiment of the invention the first heterodimeric precursor polypeptide comprises one polypeptide chain comprising a VL domain and the CH3 domain, and wherein the second heterodimeric precursor polypeptide comprises one polypeptide chain comprising a VH domain and the CH3 domain, wherein said VL domain and said VH domain specifically bind to an antigen when associated to a pair of a VH domain and a VL domain.

In one embodiment the activatable antigen binding site, e.g. a pair of antibody variable domains, is stabilized by an interchain disulfide bond. In one embodiment the first heterodimeric precursor polypeptide and the second heterodimeric precursor polypeptide comprise a hinge region, wherein the hinge region does not comprise an interchain disulfide bond and wherein the activatable antigen binding site is stabilized by an interchain disulfide bond.

In one embodiment the first heterodimeric precursor polypeptide comprises one polypeptide chain comprising a VL domain and a CH3 domain and one polypeptide chain comprising a VH domain and a CH3 domain, wherein the CH3 domains are associated with each other, and wherein the VH domain and the VL domain are associated with each other, and wherein a disulfide bond is formed between the VH domain and the VL domain by cysteine residues introduced at amino acid positions selected from the following pairs: VH position 44 and VL position 100, VH position 105 and VL position 43, and VH position 101 and VL position 100; in one embodiment VH position 44 and VL position 100.

In one embodiment the second heterodimeric precursor polypeptide comprises one polypeptide chain comprising a VL domain and a CH3 domain and one polypeptide chain comprising a VH domain and a CH3 domain, wherein the CH3 domains are associated with each other, and wherein the VH domain and the VL domain are associated with each other, and wherein a disulfide bond is formed between the VH domain and the VL domain by cysteine residues introduced at amino acid positions selected from the following pairs: VH position 44 and VL position 100, VH position 105 and VL position 43, and VH position 101 and VL position 100; in one embodiment VH position 44 and VL position 100.

In one embodiment the first heterodimeric precursor polypeptide comprises one polypeptide chain comprising a VL domain and a CH3 domain and one polypeptide chain comprising a VH domain and a CH3 domain, wherein the CH3 domains are associated with each other, and wherein the VH domain and the VL domain are associated with each other, and wherein a disulfide bond is formed between the VH domain and the VL domain by cysteine residues introduced at amino acid positions selected from the following pairs: VH position 44 and VL position 100, VH position 105 and VL position 43, and VH position 101 and VL position 100; and the second heterodimeric precursor polypeptide comprises one polypeptide chain comprising a VL domain and a CH3 domain and one polypeptide chain comprising a VH domain and a CH3 domain, wherein the CH3 domains are associated with each other, and wherein the VH domain and the VL domain are associated with each other, and wherein a disulfide bond is formed between the VH domain and the VL domain by cysteine residues introduced at amino acid positions selected from the following pairs: VH position 44 and VL position 100, VH position 105 and VL position 43, and VH position 101 and VL position 100; wherein the amino acid substitutions by cysteine in the VH domain of the first heterodimeric precursor polypeptide is at the same position as in the VH domain of the second heterodimeric precursor polypeptide, and wherein the amino acid substitutions by cysteine in the VL domain of the first heterodimeric precursor polypeptide is at the same position as in the VL domain of the second heterodimeric precursor polypeptide.

In one embodiment the first heterodimeric precursor polypeptide comprises one polypeptide chain comprising a VL domain and a CH3 domain and one polypeptide chain comprising a VH domain and a CH3 domain, wherein the CH3 domains are associated with each other, and wherein the VH domain and the VL domain are associated with each other, and wherein a disulfide bond is formed between the VH domain and the VL domain by cysteine residues introduced at amino acid positions selected from the following pairs: VH position 44 and VL position 100, VH position 105 and VL position 43, and VH position 101 and VL position 100; and the second heterodimeric precursor polypeptide comprises one polypeptide chain comprising a VL domain and a CH3 domain and one polypeptide chain comprising a VH domain and a CH3 domain, wherein the CH3 domains are associated with each other, and wherein the VH domain and the VL domain are associated with each other, and wherein a disulfide bond is formed between the VH domain and the VL domain by cysteine residues introduced at amino acid positions selected from the following pairs: VH position 44 and VL position 100, VH position 105 and VL position 43, and VH position 101 and VL position 100; wherein the amino acid substitutions by cysteine in the VH domain of the first heterodimeric precursor polypeptide is at the same position as in the VH domain of the second heterodimeric precursor polypeptide, and wherein the amino acid substitutions by cysteine in the VL domain of the first heterodimeric precursor polypeptide is at the same position as in the VL domain of the second heterodimeric precursor polypeptide.

In one embodiment of the invention the first heterodimeric precursor polypeptide comprises one polypeptide chain comprising from N- to C-terminal direction a VL domain and the CH3 domain, and wherein the second heterodimeric precursor polypeptide comprises one polypeptide chain comprising from N- to C-terminal direction a VH domain and the CH3 domain, wherein said VL domain and said VH domain specifically bind to an antigen when associated to a pair of a VH domain and a VL domain.

In one embodiment of the invention
a) the first heterodimeric precursor polypeptide comprises:
   a first heavy chain polypeptide comprising from N- to C-terminal direction a first VH domain, a CH1 domain, a second antibody variable domain selected from a VH domain and a VL domain, and a CH3 domain,
   a second heavy chain polypeptide comprising from N- to C-terminal direction an antibody variable domain capable of associating with the second antibody variable domain of the first heavy chain polypeptide, and a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
   a light chain polypeptide comprising from N- to C-terminal direction a first VL domain and a CL domain, wherein the first VH domain and the first VL domain are associated with each other and form an antigen binding site specifically binding to a target antigen; and wherein
b) the second heterodimeric precursor polypeptide comprises:
   a third heavy chain polypeptide comprising from N- to C-terminal direction a second VH domain, a CH1 domain, a third antibody variable domain selected from a VH domain and a VL domain, and a CH3 domain,
   a fourth heavy chain polypeptide comprising from N- to C-terminal direction an antibody variable domain capable of associating with the third antibody variable domain of the third heavy chain polypeptide, and a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
   a light chain polypeptide comprising from N- to C-terminal direction a second VL domain and a CL domain, wherein the second VH domain and the second VL domain are associated with each other and form an antigen binding site specifically binding to a target antigen; and wherein
c) either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation; and wherein
d) the variable domains of the first heavy chain polypeptide and the third heavy chain polypeptide are capable of forming an antigen binding site specifically binding to a target antigen.

In one embodiment the first heavy chain polypeptide comprises from N- to C-terminal direction a first VH domain, a CH1 domain, a second antibody variable domain selected from a VH domain and a VL domain, a peptide connector and a CH3 domain, and the second heavy chain polypeptide comprising from N- to C-terminal direction an antibody variable domain capable of associating with the second antibody variable domain of the first heavy chain polypeptide, a peptide connector and a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and the third heavy chain polypeptide comprises from N- to C-terminal direction a second VH domain, a CH1 domain, a third antibody variable domain selected from a VH domain and a VL domain, a peptide connector and a CH3 domain, and the fourth heavy chain polypeptide comprises from N- to C-terminal direction an antibody variable domain capable of associating with the third antibody variable domain of the third heavy chain polypeptide, a peptide connector and a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation. In one embodiment the peptide connectors comprised in the first, second, third and fourth heavy chain polypeptides are identical.

In one embodiment, within the first heterodimeric precursor polypeptide the second antibody variable domain comprised the first heavy chain polypeptide is derived from an antibody specifically binding to a first target antigen, and the antibody variable domain comprised in the second heavy chain polypeptide specifically binds to a second target antigen. Both variable domains are capable of associating with each other. Thus, one of the heavy chain polypeptides comprises a VH domain while the other heavy chain polypeptides comprises a VL domain. The VH domain and the VL domain are capable of associating with each other. However, a non-functional antigen binding site is formed. Thus the term “variable domains capable of associating with each other” within the context of the invention means that a pair of a VH and a VL domain is provided. In this embodiment, within the second heterodimeric precursor polypeptide the third antibody variable domain comprised the third heavy chain polypeptide is derived from an antibody specifically binding to a first target antigen (i.e. is capable of forming a functional VH/VL pair with the second variable domain comprised in the first heavy chain polypeptide of the first heterodimeric precursor polypeptide), and the antibody variable domain comprised in the fourth heavy chain polypeptide specifically binds to another, e.g. second, target antigen. The variable domains comprised in the first heavy chain polypeptide and the third heavy chain polypeptide are capable of associating with each other, i.e. one of the variable domains is a VH domain and the other one of the variable domains is a VL domain; and the variable domains comprised in the first heavy chain polypeptide and the third heavy chain polypeptide are capable of forming an antigen binding site specifically binding to a target antigen, i.e. both variable domains are derived from the same antibody specifically binding to the target antigen.

In one embodiment of the invention the first heterodimeric precursor polypeptide and the second heterodimeric precursor polypeptide comprise at least two polypeptide chains comprising from N- to C-terminal direction a CH2 domain and the CH3 domain, wherein the first heterodimeric precursor polypeptide comprises one polypeptide chain comprising from N- to C-terminal direction a VL domain, a CH2 domain and the CH3 domain, and wherein the second heterodimeric precursor polypeptide comprises one polypeptide chain comprising from N- to C-terminal direction a VH domain, a CH2 domain and the CH3 domain, wherein the VL domain and the VH domain are capable of forming an antigen binding site specifically binding to a target antigen.

In one embodiment of the invention
a) the first heterodimeric precursor polypeptide comprises:
   a first heavy chain polypeptide comprising from N- to C-terminal direction a first VH domain, a CH1 domain, a second antibody variable domain selected from a VH domain and a VL domain, a CH2 domain and a CH3 domain,
   a second heavy chain polypeptide comprising from N- to C-terminal direction an antibody variable domain capable of associating with the second antibody variable domain of the first heavy chain polypeptide, a CH2 domain and a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
   a light chain polypeptide comprising from N- to C-terminal direction a first VL domain and a CL domain, wherein the first VH domain and the first VL domain are associated with each other and form an antigen binding site specifically binding to a target antigen; and wherein
b) the second heterodimeric precursor polypeptide comprises:
   a third heavy chain polypeptide comprising from N- to C-terminal direction a second VH domain, a CH1 domain, a third antibody variable domain selected from a VH domain and a VL domain, a CH2 domain and a CH3 domain,
   a fourth heavy chain polypeptide comprising from N- to C-terminal direction an antibody variable domain capable of associating with the third antibody variable domain of the third heavy chain polypeptide, a CH2 domain and a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
   a light chain polypeptide comprising from N- to C-terminal direction a second VL domain and a CL domain, wherein the second VH domain and the second VL domain are associated with each other and form an antigen binding site specifically binding to a target antigen; and wherein
c) either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation; and wherein
d) the variable domains of the first heavy chain polypeptide and the third heavy chain polypeptide are capable of forming an antigen binding site specifically binding to a target antigen.

In one embodiment the first heavy chain polypeptide comprises from N- to C-terminal direction a first VH domain, a CH1 domain, a second antibody variable domain selected from a VH domain and a VL domain, a peptide connector, a CH2 domain and a CH3 domain, and the second heavy chain polypeptide comprising from N- to C-terminal direction an antibody variable domain capable of associating with the second antibody variable domain of the first heavy chain polypeptide, a peptide connector, a CH2 domain and a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and the third heavy chain polypeptide comprises from N- to C-terminal direction a second VH domain, a CH1 domain, a third antibody variable domain selected from a VH domain and a VL domain, a peptide connector, a CH2 domain and a CH3 domain, and the fourth heavy chain polypeptide comprises from N- to C-terminal direction an antibody variable domain capable of associating with the third antibody variable domain of the third heavy chain polypeptide, a peptide connector, a CH2 domain and a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation. In one embodiment the peptide connectors comprised in the first, second, third and fourth heavy chain polypeptides are identical.

Precursor Polypeptides Comprising a Hinge Region

In one embodiment of the invention, the first heterodimeric precursor polypeptide and the second heterodimeric precursor polypeptide comprise at least two polypeptide chains comprising from N- to C-terminal direction a hinge region and the CH3 domain.

In one embodiment of the invention, the first heterodimeric precursor polypeptide and the second heterodimeric precursor polypeptide comprise at least two polypeptide chains comprising from N- to C-terminal direction a hinge region, a CH2 domain and the CH3 domain.

In one embodiment of the invention, the first heterodimeric precursor polypeptide and the second heterodimeric precursor polypeptide comprise an interchain disulfide bond in the hinge region. Heterodimeric precursor polypeptides having a hinge region comprising at least one interchain disulfide bond undergo a polypeptide chain exchange in presence of a reducing agent. Thus, those heterodimeric precursor polypeptides are suitable for applications in which the presence of reducing agents is possible, e.g. for in vitro generation of product polypeptides or for screening purposes.

In one embodiment of the invention, the first heterodimeric precursor polypeptide and the second heterodimeric precursor polypeptide does not comprise an interchain disulfide bond in the hinge region. Heterodimeric precursor polypeptides having a hinge region without interchain disulfide bonds are capable of undergoing a polypeptide chain exchange in absence of a reducing agent. Hence, heterodimeric precursor polypeptides having a hinge region without interchain disulfide bonds are particularly suitable for applications in which the presence of reducing agents is not possible or not desired. Thus, those heterodimeric precursor polypeptides may be advantageously used in therapy.

In one embodiment of the invention, the first heterodimeric precursor polypeptide and the second heterodimeric precursor polypeptide comprise a natural hinge region, which does not form interchain disulfides. One example is the hinge region peptide derived from an antibody of IgG4 isotype.

Instead of a hinge region without interchain disulfide bonds the heterodimeric precursor polypeptides may comprise a peptide connector, connecting the (part of the) antigen binding moiety with the constant antibody domain (i. e. CH2 or CH3).

In one embodiment of the invention, no interchain disulfide bond is formed between the peptide connector comprised in the two polypeptide chains comprising the CH3 domain of the precursor polypeptide. In one embodiment of the invention, the peptide connectors comprised in the two polypeptide chains comprising the CH3 domain are identical to each other.

In one embodiment of the invention, the first heterodimeric precursor polypeptide and the second heterodimeric precursor polypeptide comprise at least two polypeptide chains comprising from N- to C-terminal direction a peptide connector and the CH3 domain.

In one embodiment of the invention, the first heterodimeric precursor polypeptide and the second heterodimeric precursor polypeptide comprise at least two polypeptide chains comprising from N- to C-terminal direction a peptide connector, a CH2 domain and the CH3 domain.

In one embodiment of the invention, the first heterodimeric precursor polypeptide comprises a first polypeptide chain comprising a first peptide connector, an antibody variable domain, optionally a CH2 domain, and the CH3 domain, and a second polypeptide chain comprising a first peptide connector, an antibody variable domain capable of associating with the antibody variable domain from the first polypeptide chain, optionally a CH2 domain, and the CH3 domain; and the second heterodimeric precursor polypeptide comprises a first polypeptide chain comprising a first peptide connector, an antibody variable domain, optionally a CH2 domain, and the CH3 domain, and a second polypeptide chain comprising a first peptide connector, an antibody variable domain capable of associating with the antibody variable domain from the first polypeptide chain, optionally a CH2 domain, and the CH3 domain.

In one embodiment of the invention, the peptide connector is a peptide of at least 15 amino acids. In another embodiment of the invention, the peptide connector is a peptide of 15-70 amino acids. In another embodiment of the invention, the peptide connector is a peptide of 20-50 amino acids. In another embodiment of the invention, the peptide connector is a peptide of 10-50 amino acids. Depending e.g. on the type of antigen to be bound by the activatable binding site, shorter (or even longer) peptide connectors may also be applicable in heterodimeric precursor polypeptides according to the invention.

In yet another embodiment of the invention, the first and second peptide connector are approximately of the length of the natural hinge region (which is for natural antibody molecules of IgG1 isotype about 15 amino acids, and for IgG3 isotype about 62 amino acids). Therefore, in one embodiment, wherein the first heterodimeric precursor polypeptide and the second heterodimeric precursor polypeptide are of IgG1 isotype, the peptide connectors are peptides of 10-20 amino acids, in one preferred embodiment of 12-17 amino acids. In another one embodiment, wherein the first heterodimeric precursor polypeptide and the second heterodimeric precursor polypeptide are of IgG3 isotype, the peptide connectors are peptides of 55-70 amino acids, in one preferred embodiment of 60-65 amino acids.

In one embodiment of the invention, the peptide connector is a glycine-serine linker. In one embodiment of the invention, the peptide connector is a peptide consisting of glycine and serine residues. In one embodiment of the invention, the glycine-serine linkers are of the structure

(GxS)n or (GxS)nGm
with G=glycine, S=serine, x=3 or 4, n=2, 3, 4, 5 or 6, and m=0, 1, 2 or 3.

In one embodiment, of above defined glycine-serine linkers, x=3, n=3, 4, 5 or 6, and m=0, 1, 2 or 3; or x=4, n=2, 3, 4 or 5 and m=0, 1, 2 or 3. In one preferred embodiment, x=4 and n=2 or 3, and m=0. In yet another preferred embodiment, x=4 and n=2. In one embodiment said peptide connector is (G₄S)₄ or (G₄S)₆.

In one embodiment of the invention, either i) the first heterodimeric precursor polypeptide comprises one polypeptide chain comprising a VL domain, a peptide connector and the CH3 domain, and wherein the second heterodimeric precursor polypeptide comprises one polypeptide chain comprising a VH domain, a peptide connector and the CH3 domain, wherein said VL domain and said VH domain specifically bind to an antigen when associated to a pair of a VH domain and a VL domain; or ii) the first heterodimeric precursor polypeptide comprises one polypeptide chain comprising a VH domain, a peptide connector and the CH3 domain, and wherein the second heterodimeric precursor polypeptide comprises one polypeptide chain comprising a VL domain, a peptide connector and the CH3 domain, wherein said VL domain and said VH domain specifically bind to an antigen when associated to a pair of a VH domain and a VL domain.

In one embodiment of the invention, either i) the first heterodimeric precursor polypeptide comprises one polypeptide chain comprising a VL domain, a peptide connector, a CH2 domain and the CH3 domain, and wherein the second heterodimeric precursor polypeptide comprises one polypeptide chain comprising a VH domain, a peptide connector, a CH2 domain and the CH3 domain, wherein said VL domain and said VH domain specifically bind to an antigen when associated to a pair of a VH domain and a VL domain; or ii) the first heterodimeric precursor polypeptide comprises one polypeptide chain comprising a VH domain, a peptide connector, a CH2 domain and the CH3 domain, and wherein the second heterodimeric precursor polypeptide comprises one polypeptide chain comprising a VL domain, a peptide connector, a CH2 domain and the CH3 domain, wherein said VL domain and said VH domain specifically bind to an antigen when associated to a pair of a VH domain and a VL domain.

D) Antibody Isotypes and Valency

In one embodiment of the invention, the precursor polypeptide comprises immunoglobulin constant regions of one or more immunoglobulin classes. Immunoglobulin classes include IgG, IgM, IgA, IgD, and IgE isotypes and, in the case of IgG and IgA, their subtypes. In one embodiment of the invention, the precursor polypeptide has a constant domain structure of an IgG type antibody.

In one embodiment of the invention the CH3 domains comprised in a precursor polypeptide are of mammalian IgG class. In one embodiment of the invention the CH3 domains comprised in a precursor polypeptide are of mammalian IgG1 subclass. In one embodiment of the invention the CH3 domains comprised in a precursor polypeptide are of mammalian IgG4 subclass.

In one embodiment of the invention the CH3 domains comprised in a precursor polypeptide are of human IgG class. In one embodiment of the invention the CH3 domains comprised in a precursor polypeptide are of human IgG1 subclass. In one embodiment of the invention the CH3 domains comprised in a precursor polypeptide are of human IgG4 subclass.

In one embodiment the constant domains of a precursor polypeptide according to the invention are of human IgG class. In one embodiment the constant domains of a precursor polypeptide according to the invention are of human IgG1 subclass. In one embodiment the constant domains of a precursor polypeptide according to the invention are of human IgG4 subclass.

In one embodiment, the precursor polypeptide is devoid of a CH4 domain.

In one embodiment of the invention the constant domains of a precursor polypeptide according to the invention are of the same immunoglobulin subclass. In one embodiment of the invention the variable domains and constant domains of a precursor polypeptide according to the invention are of the same immunoglobulin subclass.

In one embodiment of the invention the precursor polypeptide is an isolated precursor polypeptide. In one embodiment of the invention the product polypeptide is an isolated product polypeptide.

In one embodiment, a heterodimeric precursor polypeptide or a heterodimeric product polypeptide comprising a polypeptide chain including a CH3 domain includes a full length CH3 domain or a CH3 domain, wherein one or two C-terminal amino acid residues, i. e. G446 and/or K447 are not present.

In one embodiment the first heterodimeric precursor polypeptide is monospecific. In one embodiment the second heterodimeric precursor polypeptide is monospecific. In one embodiment the heterodimeric product polypeptide is bispecific.

In one embodiment the first heterodimeric precursor polypeptide is monospecific and comprises a part of a second antigen binding site; the second heterodimeric precursor polypeptide is monospecific and comprises the other part of the second antigen binding site. In said embodiment the heterodimeric product polypeptide is trispecific.

In one embodiment the first heterodimeric precursor polypeptide is bispecific. In one embodiment the second heterodimeric precursor polypeptide is monospecific. In one embodiment the heterodimeric product polypeptide is trispecific.

In one embodiment the first heterodimeric precursor polypeptide is bispecific. In one embodiment the second heterodimeric precursor polypeptide is bispecific. In one embodiment the heterodimeric product polypeptide is tetraspecific.

In one embodiment the first heterodimeric precursor polypeptide is monovalent. In one embodiment the second heterodimeric precursor polypeptide is monovalent.

In one embodiment the first heterodimeric precursor polypeptide is bivalent. In one embodiment the second heterodimeric precursor polypeptide is bivalent.

In one embodiment the first heterodimeric precursor polypeptide is trivalent. In one embodiment the second heterodimeric precursor polypeptide is trivalent.

In one embodiment the heterodimeric product polypeptide is bivalent. In one embodiment the heterodimeric product polypeptide is trivalent. In one embodiment the heterodimeric product polypeptide is tetravalent.

E) Further Components of Precursor Polypeptides

In one embodiment of the invention, either

i) within the first heterodimeric precursor polypeptide the polypeptide chain comprising the CH3 domain with the knob mutation and within the second heterodimeric precursor polypeptide the polypeptide chain comprising the CH3 domain with the hole mutation; or

ii) within the first heterodimeric precursor polypeptide the polypeptide chain comprising the CH3 domain with the hole mutation and within the second heterodimeric precursor polypeptide the polypeptide chain comprising the CH3 domain with the knob mutation

comprise a tagging moiety. In one embodiment the tagging moiety is fused to the C-terminus of the CH3 domains. In one embodiment the tagging moiety is an affinity tag. In one embodiment the tagging moiety is a poly(his) tag, a C-tag or a Strep-tag. In one embodiment the tagging moiety is a poly(his) tag or a C-tag.

F) Method of Generating a Product Polypeptide

In one aspect the invention provides a method of generating a heterodimeric product polypeptide, the method comprising the steps of
a) contacting a first heterodimeric precursor polypeptide and a second heterodimeric precursor polypeptide according to the invention to form a third heterodimeric polypeptide comprising at least one polypeptide chain comprising a CH3 domain from the first heterodimeric precursor polypeptide and at least one polypeptide chain comprising a CH3 domain from the second heterodimeric polypeptide, and
b) recovering the third heterodimeric polypeptide.

In one embodiment the first heterodimeric precursor polypeptide and the second heterodimeric precursor polypeptide according to the invention are contacted to form a third heterodimeric polypeptide comprising at least one polypeptide chain comprising a CH3 domain from the first heterodimeric precursor polypeptide and at least one polypeptide chain comprising a CH3 domain from the second heterodimeric polypeptide, and a fourth heterodimeric polypeptide comprising the other polypeptide comprising a CH3 domain from the first heterodimeric precursor polypeptide and the other polypeptide comprising a CH3 domain from the second heterodimeric precursor polypeptide. In one embodiment the method includes the step of recovering the fourth heterodimeric product polypeptide.

In one embodiment of the invention the method includes the formation of a third heterodimeric product polypeptide and a fourth heterodimeric product polypeptide, wherein one of the product polypeptides does not comprise an antigen binding site specifically binding to an antigen.

In one embodiment of the invention the first heterodimeric precursor polypeptide comprises an antigen binding moiety specifically binding to a first antigen, wherein the second heterodimeric precursor polypeptide comprises an antigen binding moiety specifically binding to a second antigen, and wherein the third heterodimeric polypeptide comprises the antigen binding moiety specifically binding to the first antigen and the antigen binding moiety specifically binding to the second antigen.

In one embodiment the step of contacting is carried out in a liquid solution. In one embodiment the step of contacting is carried out in a buffer solution.

In one embodiment the step of recovering is carried out by chromatography.

Reduction of Hinge Disulfides

In one embodiment of the invention the first heterodimeric precursor polypeptide and the second heterodimeric precursor polypeptide comprise a hinge region comprising an interchain disulfide bond, and the first heterodimeric precursor polypeptide and the second heterodimeric precursor polypeptide are contacted in presence of a reducing agent. In case the precursor polypeptides comprise hinge regions with interchain disulfides the polypeptide chain exchange occurs only after reduction of the interchain disulfide by a reducing agent.

In one embodiment of the invention the first heterodimeric precursor polypeptide and the second heterodimeric precursor polypeptide a hinge region that does not comprise an interchain disulfide bond. In this case, the polypeptide chain exchange may occur in absence of a reducing agent. Thus, in one embodiment the first heterodimeric precursor polypeptide and the second heterodimeric precursor polypeptide a hinge region that does not comprise an interchain disulfide bond, and the first heterodimeric precursor polypeptide and the second heterodimeric precursor polypeptide are contacted in absence of a reducing agent.

Purification

In one embodiment either i) within the first heterodimeric precursor polypeptide the polypeptide chain comprising the CH3 domain with the knob mutation and within the second heterodimeric precursor polypeptide the polypeptide chain comprising the CH3 domain with the hole mutation; or ii) within the first heterodimeric precursor polypeptide the polypeptide chain comprising the CH3 domain with the hole mutation and within the second heterodimeric precursor polypeptide the polypeptide chain comprising the CH3 domain with the knob mutation comprise a tagging moiety as defined above, and the method comprises the step of recovering the third heterodimeric polypeptide via a tag-specific affinity chromatography. In one alternative embodiment the method comprises the step of recovering the fourth heterodimeric polypeptide via a tag-specific affinity chromatography.

In one embodiment the tagging moiety is a poly(his) tag and the method includes the step of purifying the polypeptide via metal chelate affinity chromatography. In one embodiment the tagging moiety is a poly(his) tag and the method includes the step of purifying the polypeptide via nickel chelate affinity chromatography.

In one embodiment the tagging moiety is a C-tag and the method includes the step of purifying the polypeptide via C-tag affinity chromatography.

In one embodiment of the invention no interchain disulfide bond is formed between the two polypeptide chains comprising the CH3 domains of the first and second heterodimeric polypeptide, and the contacting is performed in absence of a reducing agent.

G) Heterodimeric Product Polypeptide

One aspect of the invention is a heterodimeric product polypeptide obtained by a method of generating a heterodimeric product polypeptide of the invention.

One aspect of the invention is a heterodimeric polypeptide, in one embodiment a heterodimeric product polypeptide, comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation; wherein the heterodimeric polypeptide comprises a first antigen binding moiety, wherein at least a part of the first antigen binding moiety is arranged on one of the two polypeptide chains comprising the CH3 domain; and wherein the heterodimeric polypeptide comprises a second antigen binding moiety, wherein at least a part of the second antigen binding moiety is arranged on the other one of the two polypeptide chains comprising the CH3 domain; and

wherein the CH3 domain with the hole mutation comprises at least one amino acid substitution selected from the group of:
replacement of S354 with a hydrophobic amino acid;
replacement of D356 with a positively charged amino acid;
replacement of E357 with a positively charged amino acid or with a hydrophobic amino acid;
replacement of D356 with a positively charged amino acid, and replacement of E357 with a positively charged amino acid or with a hydrophobic amino acid;
replacement of S364 with a hydrophobic amino acid;
replacement of A368 with a hydrophobic amino acid;
replacement of E392 with a negatively charged amino acid;
replacement of T394 with a hydrophobic amino acid;
replacement of D399 with a hydrophobic amino acid and replacement of S400 with a positively charged amino acid;
replacement of D399 with a hydrophobic amino acid and replacement of F405 with a positively charged amino acid;
replacement of V407 with a hydrophobic amino acid; and
replacement of K409 with a negatively charged amino acid; and
replacement of K439 with a negatively charged amino acid; and wherein

the CH3 domain with the knob mutation comprises at least one amino acid substitution selected from the group of
replacement of Q347 with a positively charged amino acid, and replacement of K360 with a negatively charged amino acid;
replacement of Y349 with a negatively charged amino acid;
replacement of L351 with a hydrophobic amino acid, and replacement of E357 with a hydrophobic amino acid;
replacement of S364 with a hydrophobic amino acid;
replacement of W366 with a hydrophobic amino acid, and replacement of K409 with a negatively charged amino acid;
replacement of L368 with a hydrophobic amino acid;
replacement of K370 with a negatively charged amino acid;
replacement of K370 with a negatively charged amino acid, and replacement of K439 with a negatively charged amino acid;
replacement of K392 with a negatively charged amino acid;
replacement of T394 with a hydrophobic amino acid;
replacement of V397 with a hydrophobic amino acid;
replacement of D399 with a positively charged amino acid, and replacement of K409 with a negatively charged amino acid;
replacement of S400 with a positively charged amino acid;
F405W;
Y407W; and
replacement of K439 with a negatively charged amino acid.

The heterodimeric (product) polypeptide according to the invention comprises two polypeptide chains comprising a CH3 domain, wherein both CH3 domains comprise the destabilizing mutations defined above. All embodiments listed above for the destabilizing mutations in the heterodimeric precursor polypeptides of the invention apply to the heterodimeric product polypeptide, with the difference that the heterodimeric product polypeptide comprises the destabilizing mutations in both CH3 domains.

Another product of the method of generating a heterodimeric product polypeptide, and therefore another aspect of the invention, is a heterodimeric product polypeptide, preferably obtained by the method of the invention, comprising two polypeptide chains comprising a CH3 domain, wherein both of the CH3 domains do not comprise a destabilizing mutation.

In one embodiment of the invention the heterodimeric product polypeptide comprises two polypeptide chains comprising a CH3 domain, wherein both CH3 domains comprise the cysteine mutations defined above. In one embodiment of the invention the heterodimeric product polypeptide comprises two polypeptide chains comprising a CH3 domain, wherein both CH3 domains do not comprise cysteine mutations as defined above.

H) Method for Generating a Multispecific Polypeptide

In one aspect the invention provides a method for identifying a multispecific heterodimeric polypeptide comprising the steps of
a) generating a plurality of multispecific heterodimeric polypeptides by subjecting each combination of
   a first heterodimeric precursor polypeptide from a plurality of first heterodimeric precursor polypeptides comprising an antigen binding moiety specifically binding to a first antigen, and
   a second heterodimeric precursor polypeptide from a plurality of second heterodimeric precursor polypeptides comprising an antigen binding moiety specifically binding to a second antigen,
   to a method of generating a heterodimeric product polypeptide according to the invention; and
b) individually detecting a desired characteristic of each multispecific heterodimeric polypeptide from the plurality of multispecific heterodimeric polypeptide generated in step a), and
c) selecting a multispecific heterodimeric polypeptide.

In one embodiment of the invention, the multispecific heterodimeric polypeptide specifically binds to the first antigen and specifically binds to the second antigen.

In one embodiment multispecific heterodimeric polypeptide is selected based on the desired characteristic detected in step b).

In one embodiment the desired characteristic that is detected in step b) is the affinity of binding of the multispecific heterodimeric polypeptide to the first antigen. In one embodiment the desired characteristic that is detected in step b) is the affinity of binding of the multispecific heterodimeric polypeptide to the second antigen. In one embodiment the desired characteristic that is detected in step b) is the thermostability of the multispecific heterodimeric polypeptide.

Another aspect of the invention is a multispecific heterodimeric polypeptide obtained by a method for identifying a multispecific heterodimeric polypeptide of the invention.

I) Recombinant Methods

Precursor polypeptides according to the invention are prepared by recombinant methods. Thus, the invention also relates to a method for the preparation of a heterodimeric precursor polypeptide according to the invention, comprising culturing a host cell comprising a nucleic acid encoding for the heterodimeric precursor polypeptide under conditions suitable for the expression of the precursor polypeptide.

In one aspect, a method of making a heterodimeric precursor polypeptide of the invention is provided, wherein the method comprises culturing a host cell comprising nucleic acid(s) encoding the heterodimeric precursor polypeptide, as provided above, under conditions suitable for expression of the heterodimeric precursor polypeptide, and optionally recovering the heterodimeric precursor polypeptide from the host cell (or host cell culture medium).

In one embodiment the method comprises the steps of transforming a host cell with expression vectors comprising nucleic acids encoding the heterodimeric precursor polypeptide, culturing said host cell under conditions that allow synthesis of said heterodimeric precursor polypeptide, and recovering said heterodimeric precursor polypeptide from said host cell culture.

For recombinant production of a heterodimeric precursor polypeptide, nucleic acids encoding the heterodimeric precursor polypeptide, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the polypeptide chains of the heterodimeric precursor polypeptide) or produced by recombinant methods or obtained by chemical synthesis.

Suitable host cells for cloning or expression of antibody-encoding vectors include prokaryotic or eukaryotic cells described herein. For example, heterodimeric precursor polypeptides may be produced in bacteria. For expression of polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, N.J. (2003), pp. 245-254, describing expression of antibody fragments in E. coli.) After expression, the heterodimeric precursor polypeptide may be isolated from the bacterial cell paste in a soluble fraction and can be further purified.

In addition to prokaryotes, eukaryotic microbes such as filamentous fungi or yeast are suitable cloning or expression hosts for vectors encoding for heterodimeric precursor polypeptides of the invention, including fungi and yeast strains whose glycosylation pathways have been “humanized”, resulting in the production of a polypeptide with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) heterodimeric precursor polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004), pp. 255-268.

In one aspect, the host cell is eukaryotic, e.g., a Chinese Hamster Ovary (CHO) cell or lymphoid cell (e.g., Y0, NS0, Sp20 cell).

In one aspect the invention provides an isolated nucleic acid encoding for a heterodimeric precursor polypeptide of the invention. In one aspect the invention provides an expression vector comprising a nucleic acid according to the invention. In another aspect the invention provides a host cell comprising the nucleic acid of the invention.

J) Therapeutic Application

The set of heterodimeric precursor polypeptides of the invention may be used in therapy. Thus, one aspect of the invention is the set of heterodimeric precursor polypeptides according to the invention for use as a medicament. Another aspect of the invention is a pharmaceutical composition comprising the set of heterodimeric precursor polypeptides of the invention and a pharmaceutically acceptable carrier. Another aspect of the invention is a method of treating an individual having a disease comprising administering to the individual an effective amount of the first and second heterodimeric precursor polypeptide of the invention or the pharmaceutical composition of the invention.

In one embodiment the heterodimeric precursor polypeptides used in therapy comprise a hinge region as defined above, wherein the first heterodimeric precursor polypeptide and the second heterodimeric precursor polypeptide do not comprise an interchain disulfide bond in the hinge region. In absence of interchain disulfide bonds in the hinge region the polypeptide chain exchange occurs in absence of a reducing agent and thus, may occur spontaneously; e.g. when both heterodimeric precursor polypeptides have bound to a target antigen or target cell.

In one embodiment heterodimeric precursor polypeptides used in therapy comprise an activatable antigen binding site as defend above.

Hence, in one embodiment the heterodimeric precursor polypeptides used in therapy comprise a hinge region, which does not comprise an interchain disulfide bond; and an activatable antigen binding site as defined above.

### 3. Specific Embodiments of the Invention

In the following specific embodiments of the invention are listed.
1. A set of heterodimeric precursor polypeptides comprising:
   a) a first heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation,
      wherein the first heterodimeric precursor polypeptide comprises a first antigen binding moiety, wherein at least a part of the first antigen binding moiety is arranged on one of the two polypeptide chains comprising the CH3 domain, and
   b) a second heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation,
      wherein the second heterodimeric precursor polypeptide comprises a second antigen binding moiety, wherein at least a part of the second antigen binding moiety is arranged on one of the two polypeptide chains comprising the CH3 domain;
   wherein
   A) either i) within the first heterodimeric precursor polypeptide the polypeptide chain comprising the CH3 domain comprising the knob mutation comprises at least a part of the first antigen binding moiety and within the second heterodimeric precursor polypeptide the polypeptide chain comprising the CH3 domain with the hole mutation comprises at least a part of the second antigen binding moiety, or ii) within the first heterodimeric precursor polypeptide the polypeptide chain comprising the CH3 domain comprising the hole mutation comprises at least a part of the first antigen binding moiety and within the second heterodimeric precursor polypeptide the polypeptide chain comprising the CH3 domain with the knob mutation comprises at least a part of the second antigen binding moiety; and wherein
   B) either
      i) the CH3 domain of the first heterodimeric precursor polypeptide comprising the knob mutation and the CH3 domain of the second heterodimeric precursor polypeptide comprising the hole mutation, or
      ii) the CH3 domain of the first heterodimeric precursor polypeptide comprising the hole mutation and the CH3 domain of the second heterodimeric precursor polypeptide comprising the knob mutation comprise the following amino acid substitutions, wherein the numbering is according to the Kabat numbering system:
      the CH3 domain with the hole mutation comprises at least one amino acid substitution selected from the group of:
         replacement of S354 with a hydrophobic amino acid;
         replacement of D356 with a positively charged amino acid;
         replacement of E357 with a positively charged amino acid or with a hydrophobic amino acid;
         replacement of D356 with a positively charged amino acid, and replacement of E357 with a positively charged amino acid or with a hydrophobic amino acid;
         replacement of S364 with a hydrophobic amino acid;
         replacement of A368 with a hydrophobic amino acid;
         replacement of E392 with a negatively charged amino acid;
         replacement of T394 with a hydrophobic amino acid;
         replacement of D399 with a hydrophobic amino acid and replacement of S400 with a positively charged amino acid;
         replacement of D399 with a hydrophobic amino acid and replacement of F405 with a positively charged amino acid;
         replacement of V407 with a hydrophobic amino acid; and
         replacement of K409 with a negatively charged amino acid; and
         replacement of K439 with a negatively charged amino acid;
      the CH3 domain with the knob mutation comprises at least one amino acid substitution selected from the group of:
         replacement of Q347 with a positively charged amino acid, and replacement of K360 with a negatively charged amino acid;
         replacement of Y349 with a negatively charged amino acid;
         replacement of L351 with a hydrophobic amino acid, and replacement of E357 with a hydrophobic amino acid;
         replacement of S364 with a hydrophobic amino acid;
         replacement of W366 with a hydrophobic amino acid, and replacement of K409 with a negatively charged amino acid;
         replacement of L368 with a hydrophobic amino acid;
         replacement of K370 with a negatively charged amino acid;
         replacement of K370 with a negatively charged amino acid, and replacement of K439 with a negatively charged amino acid;
         replacement of K392 with a negatively charged amino acid;
         replacement of T394 with a hydrophobic amino acid;
         replacement of V397 with a hydrophobic amino acid;
         replacement of D399 with a positively charged amino acid, and replacement of K409 with a negatively charged amino acid;
         replacement of S400 with a positively charged amino acid;
         F405W;
         Y407W; and
         replacement of K439 with a negatively charged amino acid.
2. The set of heterodimeric polypeptides according to embodiment 1, wherein the CH3 domains indicated in B) comprise the following amino acid substitutions:
   the CH3 domain with the hole mutation comprises at least one amino acid substitution selected from the group of S354V, S354I, S354L, D356K, D356R, E357K, E357R, E357F, S364L, S364I, A368F, K392D, K392E, T394L, T394I, V407Y, K409E, K409D, K439D, K439E and a double mutation D399A S400K, D399A S400R, D399A F405W; and
   the CH3 domain with the knob mutation comprises at least one amino acid substitution selected from the group of Y349E, Y349D, S364V, S364I, S364L, L368F, K370E, K370D, K392E, K392D, T394L, T394I, V397Y, S400K, S400R, F405W, Y407W, K349E, K439D and double mutations Q347K K360E, Q347R K360E, Q347K K360D, Q347R K360D, L351F E357F, W366I K409E, W366L K409E, W366K K409D, W366L K409D, D399K K409E, D399R K409E, D399K K409D, and D399K K409E.
3. The set of heterodimeric polypeptides according to embodiment 1 or 2, wherein the CH3 domains indicated in B) comprise the following amino acid substitutions:
   the CH3 domain with the hole mutation comprises at least one amino acid substitution selected from the group of S354V, D356K, E357K, E357F, S364L, A368F, K392E, T394I, V407Y, K409E, K439E and a double mutation D399A S400K; and
   the CH3 domain with the knob mutation comprises at least one amino acid substitution selected from the group of Y349E, S364V, L368F, K370E, K392D, T394I, V397Y, S400K, F405W, Y407W, K349E, and double mutations Q347K K360E, L351F E357F, W366I K409E, and D399K K409E.
4. The set of heterodimeric polypeptides according to one of embodiments 1 to 3, wherein the CH3 domains indicated in B) comprise the following amino acid substitutions:
   the CH3 domain with the hole mutation comprises at least one amino acid substitution selected from the group of D356K, D356R, E357K, E357R, E357F, S364L, S364I, V407Y, K409E, K409D and a double mutation D399A S400K, D399A S400R; and
   the CH3 domain with the knob mutation comprises at least one amino acid substitution selected from the group of Y349E, Y349D, K370E, K370D, K392E, K392D, T394L, T394I, V397Y, F405W, Y407W, K349E, K439D and double mutations Q347K K360E, Q347R K360E, Q347K K360D, Q347R K360D, W366I K409E, W366L K409E, W366K K409D, W366L K409D, D399K K409E, D399R K409E, D399K K409D, and D399K K409E.
5. The set of heterodimeric polypeptides according to one of embodiments 1 to 4, wherein the CH3 domains indicated in B) comprise the following amino acid substitutions:
   the CH3 domain with the hole mutation comprises at least one amino acid substitution selected from the group of D356K, E357K, E357F, S364L, V407Y, K409E, and a double mutation D399A S400K; and
   the CH3 domain with the knob mutation comprises at least one amino acid substitution selected from the group of Y349E, K370E, K392D, T394I, V397Y, F405W, Y407W, K349E, and double mutations Q347K K360E, W366I K409E, and D399K K409E.
6. The set of heterodimeric polypeptides according to one of the preceding embodiments, wherein the CH3 domain comprising the knob mutation and the CH3 domain comprising the hole mutation indicated in B) comprise one of the amino acid substitutions selected from the group indicated in the following table:

| CH3 domain comprising | CH3 domain comprising |
|---|---|
| hole mutation | knob mutation |
| D356K | K370E K439E |
| D356K E357K | Y349E |
| D356K E357K | W366I K409E |
| D356K E357K | K370E K439E |
| D356K E357K | K392D |
| D356K E357K | T394I |
| D356K E357K | D399K K409E |
| D356K E357K | F405W |
| D356K E357K | Y407W |
| E357K | Y349E |
| E357K | W366I K409E |
| E357K | K370E K439E |
| E357K | T394I |
| E357K | D399K K409E |
| E357K | F405W |
| E357K | Y407W |
| E357F | Q347K K360E |
| E357F | Y349E |
| E357F | W366I K409E |
| E357F | K370E K439E |
| E357F | T394I |
| E357F | D399K K409E |
| E357F | Y407W |
| S364L | Y349E |
| S364L | K392D |
| S364L | F405W |
| S364L | W366I K409E |
| S364L | K370E K439E |
| S364L | T394I |
| S364L | D399K K409E |
| S364L | Y407W |
| A368F | T394I |
| D399A S400K | W366I K409E |
| D399A S400K | T394I |
| V407Y | Q347K K360E |
| V407Y | Y349E |
| V407Y | W366I K409E |
| V407Y | T394I |
| V407Y | D399K K409E |
| V407Y | F405W |
| V407Y | Y407W |
| V407Y | K392D |
| K409E | Y349E |
| K409E | W366I K409E |
| K409E | K392D |
| K409E | T394I |
| K409E | D399K K409E |
| K409E | F405W |
| K409E | Y407W |

7. The set of heterodimeric polypeptides according to one of the preceding embodiments, wherein the CH3 domain comprising the knob mutation and the CH3 domain comprising the hole mutation indicated in B) comprise one of the amino acid substitutions selected from the group indicated in the following table:

| hole | knob |
|---|---|
| V407Y | K370E |
| V407Y | K370E K439E |
| V407Y | V397Y |
| S364L | K439E |
| S364L | W366I K409E |
| S364L | K370E K439E |
| S364L | D399K K409E |
| S364L | T394I |
| S364L | W366I K409E |
| S364L | K370E K439E |
| S364L | D399K K409E |
| S364L | T394I |

8. The set of heterodimeric polypeptides according to one of the preceding embodiments, wherein in case the CH3 domain with the knob mutation indicated in B) comprises a mutation E357K, the CH3 domain with the hole mutation indicated in B) does not comprise a mutation K370E.
9. The set of heterodimeric polypeptides according to one of the preceding embodiments, wherein in case the CH3 domain with the knob mutation indicated in B) comprises a mutation D356K, the CH3 domain with the hole mutation indicated in B) does not comprise a mutation K439E.
10. The set of heterodimeric polypeptides according to one of the preceding embodiments, wherein either i) the CH3 domain comprising the knob mutation of the first heterodimeric precursor polypeptide comprises a cysteine mutation and the CH3 domain comprising the hole mutation of the second heterodimeric precursor polypeptide comprises a cysteine mutation, or ii) the CH3 domain comprising the hole mutation of the first heterodimeric precursor polypeptide comprises a cysteine mutation and the CH3 domain comprising the knob mutation of the second heterodimeric precursor polypeptide comprises a cysteine mutation.
11. The set of heterodimeric polypeptides according to embodiment 10, wherein i) the CH3 domain comprising the knob mutation of the first heterodimeric precursor polypeptide comprises a substitution S354C and the CH3 domain comprising the hole mutation of the second heterodimeric precursor polypeptide comprises a substitution Y349C, or ii) the CH3 domain comprising the hole mutation of the first heterodimeric precursor polypeptide comprises a substitution Y349C and the CH3 domain comprising the knob mutation of the second heterodimeric precursor polypeptide comprises a substitution S354C.
12. The set of heterodimeric polypeptides according to embodiment 10 or 11, wherein within the first heterodimeric precursor polypeptide the CH3 domain comprising the knob mutation comprises a substitution S354C and the CH3 domain comprising the hole mutation comprises Y at position 349; and wherein within the second heterodimeric precursor polypeptide the CH3 domain comprising the hole mutation comprises a substitution Y349C and the CH3 domain comprising the knob mutation comprises S at position 354.
13. The set of heterodimeric polypeptides according to one of the preceding embodiments, wherein the first antigen binding moiety and/or the second antigen binding moiety comprise a pair of a VH domain and a VL domain, which form an antigen binding site specifically binding to a target antigen.
14. The set of heterodimeric polypeptides according to one of the preceding embodiments, wherein the first antigen binding moiety and/or the second antigen binding moiety is an antibody fragment.
15. The set of heterodimeric polypeptides according to one of the preceding embodiments, wherein
   a) the first heterodimeric precursor polypeptide comprises:
      a first heavy chain polypeptide comprising a CH3 domain and a first antibody variable domain,
      a second heavy chain polypeptide comprising a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
      a light chain polypeptide comprising a second antibody variable domain, wherein the first and second antibody variable domain together form a first antigen binding site specifically binding to a target antigen; and wherein
   b) the second heterodimeric precursor polypeptide comprises:
      a third heavy chain polypeptide comprising a CH3 domain and a third antibody variable domain,
      a fourth heavy chain polypeptide comprising a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
      a light chain polypeptide comprising a fourth antibody variable domain, wherein the third and fourth antibody variable domain together form a second antigen binding site specifically binding to a target antigen; and wherein
   c) either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation.
16. The set of heterodimeric polypeptides according to one of the preceding embodiments, wherein the first heterodimeric precursor polypeptide and the second heterodimeric precursor polypeptide comprise at least two polypeptide chains comprising a CH2 domain and the CH3 domain.
17. The set of heterodimeric polypeptides according to one of the preceding embodiments, wherein the first heterodimeric precursor polypeptide and the second heterodimeric precursor polypeptide comprise at least two polypeptide chains comprising from N- to C-terminal direction a CH2 domain and the CH3 domain.
18. The set of heterodimeric polypeptides according to one of the preceding embodiments, wherein the first heterodimeric precursor polypeptide and the second heterodimeric precursor polypeptide comprise at least two polypeptide chains comprising from N- to C-terminal direction a hinge region, a CH2 domain and the CH3 domain.
19. The set of heterodimeric polypeptides according to any one of embodiments 15 to 18, wherein
   a) the first heterodimeric precursor polypeptide comprises:
      a first heavy chain polypeptide comprising from N- to C-terminal direction a first VH domain, and constant antibody domains CH1, CH2 and CH3,
      a second heavy chain polypeptide comprising from N- to C-terminal direction constant antibody domains CH2 and CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
      a light chain polypeptide comprising from N- to C-terminal direction a first VL domain and a CL domain, wherein the first VH domain and the first VL domain are associated with each other and form an antigen binding site specifically binding to a target antigen; and wherein
   b) the second heterodimeric precursor polypeptide comprises:
      a third heavy chain polypeptide comprising from N- to C-terminal direction a second VH domain, and constant antibody domains CH1, CH2 and CH3,
      a fourth heavy chain polypeptide comprising from N- to C-terminal direction constant antibody domains CH2 and CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
      a light chain polypeptide comprising from N- to C-terminal direction a second VL domain and a CL domain, wherein the second VH domain and the second VL domain are associated with each other and form an antigen binding site specifically binding to a target antigen; and wherein
   either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation.
20. The set of heterodimeric polypeptides according to embodiment 19, wherein the first heterodimeric precursor polypeptide and the second heterodimeric precursor polypeptide comprise an interchain disulfide bond in the hinge region.
21. The set of heterodimeric polypeptides according to one of the preceding embodiments, wherein the antigen binding site is an antibody fragment.
22. The set of heterodimeric polypeptides according to one of the preceding embodiments, wherein either
   i) within the first heterodimeric precursor polypeptide the polypeptide chain comprising the CH3 domain with the knob mutation and within the second heterodimeric precursor polypeptide the polypeptide chain comprising the CH3 domain with the hole mutation; or
   ii) within the first heterodimeric precursor polypeptide the polypeptide chain comprising the CH3 domain with the hole mutation and within the second heterodimeric precursor polypeptide the polypeptide chain comprising the CH3 domain with the knob mutation comprise a tagging moiety.
23. The set of heterodimeric polypeptides according to embodiment 22, wherein the tagging moiety is fused to the C-terminus of the CH3 domains.
24. The set of heterodimeric polypeptides according to embodiment 22 or 23, wherein the tagging moiety is a histidine tag or a C-tag.
25. The set of heterodimeric polypeptides according to one of the preceding embodiments, wherein the first heterodimeric precursor polypeptide comprises one polypeptide chain comprising a VL domain and the CH3 domain, and wherein the second heterodimeric precursor polypeptide comprises one polypeptide chain comprising a VH domain and the CH3 domain, wherein said VL domain and said VH domain specifically bind to an antigen when associated to a pair of a VH domain and a VL domain.
26. The set of heterodimeric polypeptides according to one of the preceding embodiments, wherein
   a) the first heterodimeric precursor polypeptide comprises:
      a first heavy chain polypeptide comprising from N- to C-terminal direction a first VH domain, a CH1 domain, a second antibody variable domain selected from a VH domain and a VL domain, and a CH3 domain,
      a second heavy chain polypeptide comprising from N- to C-terminal direction an antibody variable domain capable of associating with the second antibody variable domain of the first heavy chain polypeptide, and a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
      a light chain polypeptide comprising from N- to C-terminal direction a first VL domain and a CL domain, wherein the first VH domain and the first VL domain are associated with each other and form an antigen binding site specifically binding to a target antigen; and wherein
   b) the second heterodimeric precursor polypeptide comprises:
      a third heavy chain polypeptide comprising from N- to C-terminal direction a second VH domain, a CH1 domain, a third antibody variable domain selected from a VH domain and a VL domain, and a CH3 domain,
      a fourth heavy chain polypeptide comprising from N- to C-terminal direction an antibody variable domain capable of associating with the third antibody variable domain of the third heavy chain polypeptide, and a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
      a light chain polypeptide comprising from N- to C-terminal direction a second VL domain and a CL domain, wherein the second VH domain and the second VL domain are associated with each other and form an antigen binding site specifically binding to a target antigen; and wherein
   c) either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation; and wherein
   d) the variable domains of the first heavy chain polypeptide and the third heavy chain polypeptide are capable of forming an antigen binding site specifically binding to a target antigen.
27. The set of heterodimeric polypeptides according to one of the preceding embodiments,
   wherein the first heterodimeric precursor polypeptide and the second heterodimeric precursor polypeptide comprise at least two polypeptide chains comprising from N- to C-terminal direction a CH2 domain and the CH3 domain, wherein the first heterodimeric precursor polypeptide comprises one polypeptide chain comprising from N- to C-terminal direction a VL domain, a CH2 domain and the CH3 domain, and wherein the second heterodimeric precursor polypeptide comprises one polypeptide chain comprising from N- to C-terminal direction a VH domain, a CH2 domain and the CH3 domain, wherein the VL domain and the VH domain are capable of forming an antigen binding site specifically binding to a target antigen.
28. The set of heterodimeric polypeptides according to one of the preceding embodiments, wherein
   a) the first heterodimeric precursor polypeptide comprises:
      a first heavy chain polypeptide comprising from N- to C-terminal direction a first VH domain, a CH1 domain, a second antibody variable domain selected from a VH domain and a VL domain, a CH2 domain and a CH3 domain,
      a second heavy chain polypeptide comprising from N- to C-terminal direction an antibody variable domain capable of associating with the second antibody variable domain of the first heavy chain polypeptide, a CH2 domain and a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
      a light chain polypeptide comprising from N- to C-terminal direction a first VL domain and a CL domain, wherein the first VH domain and the first VL domain are associated with each other and form an antigen binding site specifically binding to a target antigen; and wherein
   b) the second heterodimeric precursor polypeptide comprises:
      a third heavy chain polypeptide comprising from N- to C-terminal direction a second VH domain, a CH1 domain, a third antibody variable domain selected from a VH domain and a VL domain, a CH2 domain and a CH3 domain,
      a fourth heavy chain polypeptide comprising from N- to C-terminal direction an antibody variable domain capable of associating with the third antibody variable domain of the third heavy chain polypeptide, a CH2 domain and a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
      a light chain polypeptide comprising from N- to C-terminal direction a second VL domain and a CL domain, wherein the second VH domain and the second VL domain are associated with each other and form an antigen binding site specifically binding to a target antigen; and wherein
   c) either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation; and wherein
   d) the variable domains of the first heavy chain polypeptide and the third heavy chain polypeptide are capable of forming an antigen binding site specifically binding to a target antigen.
29. The set of heterodimeric precursor polypeptides according to one of the preceding embodiments, wherein the antigen binding moiety of the first heterodimeric precursor polypeptide and the antigen binding moiety of the second heterodimeric precursor polypeptide bind to the same antigen.
30. The set of heterodimeric precursor polypeptides according to one of the preceding embodiments, wherein the antigen binding moiety of the first heterodimeric precursor polypeptide and the antigen binding moiety of the second heterodimeric precursor polypeptide bind to different antigens.
31. A heterodimeric polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation; wherein the heterodimeric polypeptide comprises a first antigen binding moiety, wherein at least a part of the first antigen binding moiety is arranged on one of the two polypeptide chains comprising the CH3 domain; and wherein the heterodimeric polypeptide comprises a second antigen binding moiety, wherein at least a part of the second antigen binding moiety is arranged on the other one of the two polypeptide chains comprising the CH3 domain; and
   wherein the CH3 domain with the hole mutation comprises at least one amino acid substitution selected from the group of:
      replacement of S354 with a hydrophobic amino acid;
      replacement of D356 with a positively charged amino acid;
      replacement of E357 with a positively charged amino acid or with a hydrophobic amino acid;
      replacement of D356 with a positively charged amino acid, and replacement of E357 with a positively charged amino acid or with a hydrophobic amino acid;
      replacement of S364 with a hydrophobic amino acid;
      replacement of A368 with a hydrophobic amino acid;
      replacement of E392 with a negatively charged amino acid;
      replacement of T394 with a hydrophobic amino acid;
      replacement of D399 with a hydrophobic amino acid and replacement of S400 with a positively charged amino acid;
      replacement of D399 with a hydrophobic amino acid and replacement of F405 with a positively charged amino acid;
      replacement of V407 with a hydrophobic amino acid; and
      replacement of K409 with a negatively charged amino acid; and
      replacement of K439 with a negatively charged amino acid; and wherein
   the CH3 domain with the knob mutation comprises at least one amino acid substitution selected from the group of
      replacement of Q347 with a positively charged amino acid, and replacement of K360 with a negatively charged amino acid;
      replacement of Y349 with a negatively charged amino acid;
      replacement of L351 with a hydrophobic amino acid, and replacement of E357 with a hydrophobic amino acid;
      replacement of S364 with a hydrophobic amino acid;
      replacement of W366 with a hydrophobic amino acid, and replacement of K409 with a negatively charged amino acid;
      replacement of L368 with a hydrophobic amino acid;
      replacement of K370 with a negatively charged amino acid;
      replacement of K370 with a negatively charged amino acid, and replacement of K439 with a negatively charged amino acid;
      replacement of K392 with a negatively charged amino acid;
      replacement of T394 with a hydrophobic amino acid;
      replacement of V397 with a hydrophobic amino acid;
      replacement of D399 with a positively charged amino acid, and replacement of K409 with a negatively charged amino acid;
      replacement of S400 with a positively charged amino acid;
      F405W;
      Y407W; and
      replacement of K439 with a negatively charged amino acid.
32. A method for generating a heterodimeric polypeptide comprising the steps of:
   a) contacting a first heterodimeric precursor polypeptide and a second heterodimeric precursor polypeptide, as defined in one of embodiments 1 to 30 to form a third heterodimeric polypeptide comprising at least one polypeptide chain comprising a CH3 domain from the first heterodimeric precursor polypeptide and at least one polypeptide chain comprising a CH3 domain from the second heterodimeric polypeptide, and
   b) recovering the third heterodimeric polypeptide.
33. The method according to embodiment 32, wherein the first heterodimeric precursor polypeptide comprises an antigen binding moiety specifically binding to a first antigen, wherein the second heterodimeric precursor polypeptide comprises an antigen binding moiety specifically binding to a second antigen, and wherein the third heterodimeric polypeptide comprises the antigen binding moiety specifically binding to the first antigen and the antigen binding moiety specifically binding to the second antigen.
34. The method according to one of embodiments 32 or 33, wherein
   i) within the first heterodimeric precursor polypeptide the polypeptide chain comprising the CH3 domain with the knob mutation and within the second heterodimeric precursor polypeptide the polypeptide chain comprising the CH3 domain with the hole mutation; or
   ii) within the first heterodimeric precursor polypeptide the polypeptide chain comprising the CH3 domain with the hole mutation and within the second heterodimeric precursor polypeptide the polypeptide chain comprising the CH3 domain with the knob mutation
   comprise a tagging moiety, and wherein the method comprises the step of recovering the third heterodimeric polypeptide via a tag-specific affinity chromatography.
35. The method according to one of embodiments 32 to 34, wherein the first heterodimeric precursor polypeptide and the second heterodimeric precursor polypeptide comprise an interchain disulfide bond in the hinge region, and the first heterodimeric precursor polypeptide and the second heterodimeric precursor polypeptide are contacted in presence of a reducing agent.
36. A heterodimeric polypeptide obtained by a method according to any one of embodiments 32 to 35.
37. A method for identifying a multispecific heterodimeric polypeptide comprising the steps of
   a) generating a plurality of multispecific heterodimeric polypeptides by subjecting each combination of
      a first heterodimeric precursor polypeptide from a plurality of first heterodimeric precursor polypeptides comprising an antigen binding moiety specifically binding to a first antigen, and
      a second heterodimeric precursor polypeptide from a plurality of second heterodimeric precursor polypeptides comprising an antigen binding moiety specifically binding to a second antigen, to a method according to one of embodiments 32 to 35; and
   b) individually detecting a desired characteristic of each multispecific heterodimeric polypeptide from the plurality of multispecific heterodimeric polypeptide generated in step a), and
   c) selecting a multispecific heterodimeric polypeptide.
38. The method according to embodiment 37, wherein the desired characteristic is selected from affinity and thermostability.
39. A multispecific heterodimeric polypeptide obtained by a method according to any one of embodiments 37 to 38.
40. The set of heterodimeric precursor polypeptides according to any one of embodiments 1 to 30 for use as a medicament.
41. The set of heterodimeric precursor polypeptides according to any one of embodiments 26 to 30 for use as a medicament.
42. The set of heterodimeric precursor polypeptides according to any one of embodiments 26 to 30 for use as a medicament, wherein the heterodimeric precursor polypeptides comprise a hinge region, wherein the hinge region does not comprise interchain disulfide bonds.
43. A pharmaceutical composition comprising the set of heterodimeric precursor polypeptides according to one of embodiments 1 to 30 and a pharmaceutically acceptable carrier.
44. A first heterodimeric precursor polypeptide as defined in any one of embodiments 1 to 30.
45. A second heterodimeric precursor polypeptide as defined in any one of embodiments 1 to 30.
46. Use of a heterodimeric precursor polypeptide according to one of embodiments 44 or 45 in a method according to one of embodiments 32 to 36.

DESCRIPTION OF AMINO ACID SEQUENCES

| | |
|---|---|
| SEQ ID | anti-biocytinamid VL |
| NO: 1 | DIVMTQTPLSLSVTPGQPASISCKSSQSLVHSNGNTYLRWYL |
| | QKPGQSPKVLIYKVSNRVSGVPDRFSGSGSGTDFTLKISRVE |
| | AEDVGVYYCSQSTHVPWTFGQGTKLEIK |
| SEQ ID | anti-biocytinamid VH |
| NO: 2 | GVKLDETGGGLVQPGGAMKLSCVTSGFTFGHYWMNWVRQSPE |
| | KGLEWVAQFRNKPYNYETYYSDSVKGRFTISRDDSKSSVYLQ |
| | MNNLRVEDTGIYYCTGASYGMEYLGQGTSVTVSS |
| SEQ ID | anti-biocytinamid light chain polypeptide |
| NO: 3 | DIVMTQTPLSLSVTPGQPASISCKSSQSLVHSNGNTYLRWYL |
| | QKPGQSPKVLIYKVSNRVSGVPDRFSGSGSGTDFTLKISRVE |
| | AEDVGVYYCSQSTHVPWTFGQGTKLEIKRTVAAPSVFIFPPS |
| | DEQLKSGTASVVCLLNNFYPREAKVQWKVDNALQSGNSQESV |
| | TEQDSKDSTYSLSSTLTLSKADYEKHKVYACEVTHQGLSSPV |
| | TKSFNRGEC |
| SEQ ID | anti-biocytinamid heavy chain polypeptide |
| NO: 4 | with knob |
| | GVKLDETGGGLVQPGGAMKLSCVTSGFTFGHYWMNWVRQSPE |
| | KGLEWVAQFRNKPYNYETYYSDSVKGRFTISRDDSKSSVYLQ |
| | MNNLRVEDTGIYYCTGASYGMEYLGQGTSVIVSSASTKGPSV |
| | FPLAPSSKSTSGGTAALGCLVKDYFPEPVTVSWNSGALTSGV |
| | HTFPAVLQSSGLYSLSSVVTVPSSSLGTQTYICNVNHKPSNT |
| | KVDKKVEPKSCDKTHTCPPCPAPELLGGPSVFLFPPKPKDTL |
| | MISRTPEVTCVVVDVSHEDPEVKFNWYVDGVEVHNAKTKPRE |
| | EQYNSTYRVVSVLTVLHQDWLNGKEYKCKVSNKALPAPIEKT |
| | ISKAKGQPREPQVYTLPPCRDELTKNQVSLWCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| SEQ ID | heavy chain hinge-CH2-CH3 polypeptide |
| NO: 5 | with hole and tag |
| | DKTHTCPPCPAPELLGGPSVFLFPPKPKDTLMISRTPEVTCV |
| | VVDVSHEDPEVKFNWYVDGVEVHNAKTKPREEQYNSTYRVVS |
| | VLTVLHQDWLNGKEYKCKVSNKALPAPIEKTISKAKGQPREP |
| | QVCTLPPSRDELTKNQVSLSCAVKGFYPSDIAVEWESNGQPE |
| | NNYKTTPPVLDSDGSFFLVSKLTVDKSRWQQGNVFSCSVMHE |
| | ALHNHYTQKSLSLSPGGGGGSHHHHHHHH |
| SEQ ID | anti-fluorescein VL |
| NO: 6 | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIK |
| SEQ ID | anti-fluorescein VH |
| NO: 7 | GVKLDETGGGLVQPGGAMKLSCVTSGFTFGHYWMNWVRQSPE |
| | KGLEWVAQFRNKPYNYETYYSDSVKGRFTISRDDSKSSVYLQ |
| | MNNLRVEDTGIYYCTGASYGMEYLGQGTSVTVSS |
| SEQ ID | anti-fluorescein light chain polypeptide |
| NO: 8 | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKRTVAAPSVFIFPPSDEQLK |
| | SGTASVVCLLNNFYPREAKVQWKVDNALQSGNSQESVTEQDS |
| | KDSTYSLSSILTLSKADYEKHKVYACEVTHQGLSSPVTKSFN |
| | RGEC |
| SEQ ID | anti-fluorescein heavy chain polypeptide |
| NO: 9 | with hole |
| | GVKLDETGGGLVQPGGAMKLSCVTSGFTFGHYWMNWVRQSPE |
| | KGLEWVAQFRNKPYNYETYYSDSVKGRFTISRDDSKSSVYLQ |
| | MNNLRVEDTGIYYCTGASYGMEYLGQGTSVTVSSASTKGPSV |
| | FPLAPSSKSTSGGTAALGCLVKDYFPEPVTVSWNSGALTSGV |
| | HTFPAVLQSSGLYSLSSVVTVPSSSLGTQTYICNVNHKPSNT |
| | KVDKKVEPKSCDKTHTCPPCPAPELLGGPSVFLFPPKPKDTL |
| | MISRTPEVTCVVVDVSHEDPEVKFNWYVDGVEVHNAKTKPRE |
| | EQYNSTYRVVSVLTVLHQDWLNGKEYKCKVSNKALPAPIEKT |
| | ISKAKGQPREPQVYTLPPCRDELTKNQVSLWCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| SEQ ID | heavy chain hinge-CH2-CH3 polypeptide |
| NO: 10 | with knob and tag |
| | DKTHTCPPCPAPELLGGPSVFLFPPKPKDTLMISRTPEVTCV |
| | VVDVSHEDPEVKFNWYVDGVEVHNAKTKPREEQYNSTYRVVS |
| | VLTVLHQDWLNGKEYKCKVSNKALPAPIEKTISKAKGQPREP |
| | QVYTLPPCRDELTKNQVSLWCLVKGFYPSDIAVEWESNGQPE |
| | NNYKTTPPVLDSDGSFFLYSKLTVDKSRWQQGNVFSCSVMHE |
| | ALHNHYTQKSLSLSPGGGGGSHHHHHHHH |
| SEQ ID | anti-LeY light chain polypeptide |
| NO: 11 | DVLMTQSPLSLPVSLGDQASISCRSSQIIVHSNGNTYLEWYL |
| | QKPGQSPKLLIYKVSNRFSGVPDRFSGSGSGTDFTLKISRVE |
| | AEDLGVYYCFQGSHVPFTFGSGTKLEIKRTVAAPSVFIFPPS |
| | DEQLKSGTASVVCLLNNFYPREAKVQWKVDNALQSGNSQESV |
| | TEQDSKDSTYSLSSTLTLSKADYEKHKVYACEVTHQGLSSPV |
| | TKSFNRGEC |
| SEQ ID | anti-LeY heavy chain polypeptide with |
| NO: 12 | knob and VH anti-CD3 |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGGGSEVQLVQSGAE |
| | VKKPGASVKVSCKASGYTFTNYYIHWVRQAPGQGLEWIGWIY |
| | PGDGNTKYNEKFKGRATLTADTSTSTAYLELSSLRSEDTAVY |
| | YCARDSYSNYYFDYWGQGTLVTVSSGQPREPQVYTLPPSRDE |
| | LTKNQVSLSCAVKGFYPSDIAVEWESNGQPENNYKTTPPVLD |
| | SDGSFFLVSKLTVDKSRWQQGNVFSCSVMHEALHNHYTQKSL |
| | SLSPGK |
| SEQ ID | heavy chain polypeptide with hole and |
| NO: 13 | VL and tag |
| | DIQMTQSPSSLSASVGDRVTITCRASQDIKNYLNWYQQKPGK |
| | APKLLIYYSSTLLSGVPSRFSGSGSGTDFTLTISSLQPEDFA |
| | TYYCQQSITLPPTFGGGTKVEIKGQPREPQVYTLPPSRDELT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSHHHHHHHH |
| SEQ ID | anti-LeY heavy chain polypeptide with |
| NO: 14 | hole and VL anti-CD3 |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGGGSDIVMTQSPDS |
| | LAVSLGERATINCKSSQSLLNSRTRKNYLAWYQQKPGQPPKL |
| | LIYWASTRESGVPDRFSGSGSGTDFTLTISSLQAEDVAVYYC |
| | TQSFILRTFGQGTKVEIKGQPREPQVYTLPPSRDELTKNQVS |
| | LSCAVKGFYPSDIAVEWESNGQPENNYKTTPPVLDSDGSFFL |
| | VSKLTVDKSRWQQGNVFSCSVMHEALHNHYTQKSLSLSPGK |
| SEQ ID | heavy chain polypeptide with knob and |
| NO: 15 | VH and tag |
| | EVQLVESGGGLVQPGGSLRLSCAASGFSIAGTAIHWVRQAPG |
| | KGLEWVASISPGGGSTAYADSVKGRFTISADTSKNTAYLQMN |
| | SLRAEDTAVYYCSRSGGSGASAMDYWGQGTLVTVSSGQPREP |
| | QVYTLPPSRDELTKNQVSLWCLVKGFYPSDIAVEWESNGQPE |
| | NNYKTTPPVLDSDGSFFLYSKLTVDKSRWQQGNVFSCSVMHE |
| | ALHNHYTQKSLSLSPGKGGGGSHHHHHHHH |
| SEQ ID | anti-LeY heavy chain polypeptide with |
| NO: 16 | knob and VL anti-CD3 |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGGGSDIVMTQSPDS |
| | LAVSLGERATINCKSSQSLLNSRTRKNYLAWYQQKPGQPPKL |
| | LIYWASTRESGVPDRFSGSGSGTDFTLTISSLQAEDVAVYYC |
| | TQSFILRTFGQGTKVEIKGQPREPQVYTLPPSRDELTKNQVS |
| | LSCAVKGFYPSDIAVEWESNGQPENNYKTTPPVLDSDGSFFL |
| | VSKLTVDKSRWQQGNVFSCSVMHEALHNHYTQKSLSLSPGK |
| SEQ ID | heavy chain polypeptide with hole and |
| NO: 17 | VH and tag |
| | EVQLVESGGGLVQPGGSLRLSCAASGFSIAGTAIHWVRQAPG |
| | KGLEWVASISPGGGSTAYADSVKGRFTISADTSKNTAYLQMN |
| | SLRAEDTAVYYCSRSGGSGASAMDYWGQGTLVTVSSGQPREP |
| | QVYTLPPSRDELTKNQVSLSCAVKGFYPSDIAVEWESNGQPE |
| | NNYKTTPPVLDSDGSFFLVSKLTVDKSRWQQGNVFSCSVMHE |
| | ALHNHYTQKSLSLSPGKGGGGSHHHHHHHH |
| SEQ ID | anti-LeY heavy chain polypeptide with |
| NO: 18 | hole and VH anti-CD3 |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGGGSEVQLVQSGAE |
| | VKKPGASVKVSCKASGYTFTNYYIHWVRQAPGQGLEWIGWIY |
| | PGDGNTKYNEKFKGRATLTADTSTSTAYLELSSLRSEDTAVY |
| | YCARDSYSNYYFDYWGQGTLVTVSSGQPREPQVYTLPPSRDE |
| | LTKNQVSLSCAVKGFYPSDIAVEWESNGQPENNYKTTPPVLD |
| | SDGSFFLVSKLTVDKSRWQQGNVFSCSVMHEALHNHYTQKSL |
| | SLSPGK |
| SEQ ID | heavy chain polypeptide with knob |
| NO: 19 | and VL and tag |
| | DIQMTQSPSSLSASVGDRVTITCRASQDIKNYLNWYQQKPGK |
| | APKLLIYYSSTLLSGVPSRFSGSGSGTDFTLTISSLQPEDFA |
| | TYYCQQSITLPPTFGGGTKVEIKGQPREPQVYTLPPSRDELT |
| | KNQVSLWCLVKGFYPSDIAVEWESNGQPENNYKTTPPVLDSD |
| | GSFFLYSKLTVDKSRWQQGNVFSCSVMHEALHNHYTQKSLSL |
| | SPGKGGGGSHHHHHHHH |
| SEQ ID | anti-LeY heavy chain polypeptide with |
| NO: 20 | knob and VH anti-CD3 and CH2 |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGSGGEVQLVQSGAE |
| | VKKPGASVKVSCKASGYTFTNYYIHWVRQAPGQGLEWIGWIY |
| | PGDGNTKYNEKFKGRATLTADTSTSTAYLELSSLRSEDTAVY |
| | YCARDSYSNYYFDYWGQGTLVTVSSASGGGGSGGGGSGGSGG |
| | GEAAGGPSVFLFPPKPKDTLMISRTPEVTCVVVDVSHEDPEV |
| | KFNWYVDGVEVHNAKTKPREEQYNSTYRVVSVLTVLHQDWLN |
| | GKEYKCKVSNKALGAPIEKTISKAKGQPREPQVYTLPPSRDE |
| | LTKNQVSLWCLVKGFYPSDIAVEWESNGQPENNYKTTPPVLD |
| | SDGSFFLYSKLTVDKSRWQQGNVFSCSVMHEALHNHYTQKSL |
| | SLSPGK |
| SEQ ID | heavy chain polypeptide with hole and |
| NO: 21 | VL and CH2 and tag |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKSSGGGGSGGGGSGGSGGGE |
| | AAGGPSVFLFPPKPKDTLMISRTPEVTCVVVDVSHEDPEVKF |
| | NWYVDGVEVHNAKTKPREEQYNSTYRVVSVLTVLHQDWLNGK |
| | EYKCKVSNKALGAPIEKTISKAKGQPREPQVYTLPPSRDELT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGGGGGSHHHHHHHH |
| SEQ ID | anti-LeY heavy chain polypeptide with |
| NO: 22 | hole and VL anti-CD3 and CH2 |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGSGGDIVMTQSPDS |
| | LAVSLGERATINCKSSQSLLNSRTRKNYLAWYQQKPGQPPKL |
| | LIYWASTRESGVPDRFSGSGSGTDFTLTISSLQAEDVAVYYC |
| | TQSFILRTFGQGTKVEIKSSGGGGSGGGGSGGSGGGEAAGGP |
| | SVFLFPPKPKDTLMISRTPEVTCVVVDVSHEDPEVKFNWYVD |
| | GVEVHNAKTKPREEQYNSTYRVVSVLTVLHQDWLNGKEYKCK |
| | VSNKALGAPIEKTISKAKGQPREPQVYTLPPSRDELTKNQVS |
| | LSCAVKGFYPSDIAVEWESNGQPENNYKTTPPVLDSDGSFFL |
| | VSKLTVDKSRWQQGNVFSCSVMHEALHNHYTQKSLSLSPGK |
| SEQ ID | heavy chain polypeptide with knob and |
| NO: 23 | VH and CH2 domain and tag |
| | QVQLVESGGGLVKPGGSLRLSCAASGFTFSDYAMSWIRQAPG |
| | KGLEWVSSINIGATYIYYADSVKGRFTISRDNAKNSLYLQMN |
| | SLRAEDTAVYYCARPGSPYEYDKAYYSMAYWGQGTTVTVSSA |
| | SGGGGSGGGGSGGSGGGEAAGGPSVFLFPPKPKDTLMISRTP |
| | EVTCVVVDVSHEDPEVKFNWYVDGVEVHNAKTKPREEQYNST |
| | YRVVSVLTVLHQDWLNGKEYKCKVSNKALGAPIEKTISKAKG |
| | QPREPQVYTLPPSRDELTKNQVSLWCLVKGFYPSDIAVEWES |
| | NGQPENNYKTTPPVLDSDGSFFLYSKLTVDKSRWQQGNVFSC |
| | SVMHEALHNHYTQKSLSLSPGGGGGSHHHHHHHH |
| SEQ ID | anti-LeY heavy chain polypeptide with |
| NO: 24 | knob and VL anti-CD3 and CH2 |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGSGGDIVMTQSPDS |
| | LAVSLGERATINCKSSQSLLNSRTRKNYLAWYQQKPGQPPKL |
| | LIYWASTRESGVPDRFSGSGSGTDFTLTISSLQAEDVAVYYC |
| | TQSFILRTFGQGTKVEIKSSGGGGSGGGGSGGSGGGEAAGGP |
| | SVFLFPPKPKDTLMISRTPEVTCVVVDVSHEDPEVKFNWYVD |
| | GVEVHNAKTKPREEQYNSTYRVVSVLTVLHQDWLNGKEYKCK |
| | VSNKALGAPIEKTISKAKGQPREPQVYTLPPSRDELTKNQVS |
| | LWCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID | heavy chain polypeptide with hole and |
| NO: 25 | VH and CH2 and tag |
| | QVQLVESGGGLVKPGGSLRLSCAASGFTFSDYAMSWIRQAPG |
| | KGLEWVSSINIGATYIYYADSVKGRFTISRDNAKNSLYLQMN |
| | SLRAEDTAVYYCARPGSPYEYDKAYYSMAYWGQGTIVIVSSA |
| | SGGGGSGGGGSGGSGGGEAAGGPSVFLFPPKPKDTLMISRTP |
| | EVTCVVVDVSHEDPEVKFNWYVDGVEVHNAKTKPREEQYNST |
| | YRVVSVLTVLHQDWLNGKEYKCKVSNKALGAPIEKTISKAKG |
| | QPREPQVYTLPPSRDELTKNQVSLSCAVKGFYPSDIAVEWES |
| | NGQPENNYKTTPPVLDSDGSFFLVSKLTVDKSRWQQGNVFSC |
| | SVMHEALHNHYTQKSLSLSPGGGGGSHHHHHHHH |
| SEQ ID | anti-LeY heavy chain polypeptide with |
| NO: 26 | hole and VH anti-CD3 and CH2 |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGSGGEVQLVQSGAE |
| | VKKPGASVKVSCKASGYTFTNYYIHWVRQAPGQGLEWIGWIY |
| | PGDGNTKYNEKFKGRATLTADTSTSTAYLELSSLRSEDTAVY |
| | YCARDSYSNYYFDYWGQGTLVTVSSASGGGGSGGGGSGGSGG |
| | GEAAGGPSVFLFPPKPKDTLMISRTPEVTCVVVDVSHEDPEV |
| | KFNWYVDGVEVHNAKTKPREEQYNSTYRVVSVLTVLHQDWLN |
| | GKEYKCKVSNKALGAPIEKTISKAKGQPREPQVYTLPPSRDE |
| | LTKNQVSLSCAVKGFYPSDIAVEWESNGQPENNYKTTPPVLD |
| | SDGSFFLVSKLTVDKSRWQQGNVFSCSVMHEALHNHYTQKSL |
| | SLSPGK |
| SEQ ID | heavy chain polypeptide with knob and |
| NO: 27 | VL and CH2 and tag |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKSSGGGGSGGGGSGGSGGGE |
| | AAGGPSVFLFPPKPKDTLMISRTPEVTCVVVDVSHEDPEVKF |
| | NWYVDGVEVHNAKTKPREEQYNSTYRVVSVLTVLHQDWLNGK |
| | EYKCKVSNKALGAPIEKTISKAKGQPREPQVYTLPPSRDELT |
| | KNQVSLWCLVKGFYPSDIAVEWESNGQPENNYKTTPPVLDSD |
| | GSFFLYSKLTVDKSRWQQGNVFSCSVMHEALHNHYTQKSLSL |
| | SPGGGGGSHHHHHHHH |
| SEQ ID | anti-biocytinamid heavy chain |
| NO: 28 | polypeptide with hole |
| | QVQLVQSGAEVKKPGSSVKVSCKSSGFNNKDTFFQWVRQAPG |
| | QGLEWMGRIDPANGFTKYAQKFQGRVTITADTSTSTAYMELS |
| | SLRSEDTAVYYCARWDTYGAAWFAYWGQGTLVTVSSASTKGP |
| | SVFPLAPSSKSTSGGTAALGCLVKDYFPEPVTVSWNSGALTS |
| | GVHTFPAVLQSSGLYSLSSVVTVPSSSLGTQTYICNVNHKPS |
| | NTKVDKKVEPKSCDKTHTCPPCPAPELLGGPSVFLFPPKPKD |
| | TLMISRIPEVTCVVVDVSHEDPEVKFNWYVDGVEVHNAKTKP |
| | REEQYNSTYRVVSVLTVLHQDWLNGKEYKCKVSNKALPAPIE |
| | KTISKAKGQPREPQVCTLPPSRDELTKNQVSLSCAVKGFYPS |
| | DIAVEWESNGQPENNYKTTPPVLDSDGSFFLVSKLTVDKSRW |
| | QQGNVFSCSVMHEALHNHYTQKSLSLSPGK |
| SEQ ID | anti-fluorescein heavy chain |
| NO: 29 | polypeptide with knob |
| | GVKLDETGGGLVQPGGAMKLSCVTSGFTFGHYWMNWVRQSPE |
| | KGLEWVAQFRNKPYNYETYYSDSVKGRFTISRDDSKSSVYLQ |
| | MNNLRVEDTGIYYCTGASYGMEYLGQGTSVTVSSASTKGPSV |
| | FPLAPSSKSTSGGTAALGCLVKDYFPEPVTVSWNSGALTSGV |
| | HTFPAVLQSSGLYSLSSVVTVPSSSLGTQTYICNVNHKPSNT |
| | KVDKKVEPKSCDKTHTCPPCPAPELLGGPSVFLFPPKPKDTL |
| | MISRTPEVTCVVVDVSHEDPEVKFNWYVDGVEVHNAKTKPRE |
| | EQYNSTYRVVSVLTVLHQDWLNGKEYKCKVSNKALPAPIEKT |
| | ISKAKGQPREPQVYTLPPCRDELTKNQVSLWCLVKGFYPSDI |
| | AVEWESNGQPENNYKTTPPVLDSDGSFFLYSKLTVDKSRWQQ |
| | GNVFSCSVMHEALHNHYTQKSLSLSPGK |

### EXAMPLES

The following examples are provided to aid the understanding of the present invention, the true scope of which is set forth in the appended claims. It is understood that modifications can be made in the procedures set forth without departing from the spirit of the invention.

### Example 1

### Generation of Monospecific Precursor Polypeptides Comprising a Full Fe Domain

For assessing formation of bispecific anti-biocytinamid/anti-fluorescein antibodies from monospecific precursor polypeptides, monospecific precursor polypeptides of a domain arrangement as depicted for the first and second heterodimeric precursor polypeptides indicated in FIG. 1 were generated.

The first heterodimeric precursor polypeptide (also referred to as “anti-bio precursor”) comprised a Fab fragment specifically binding to biocytinamid (“bio”), a biotin derivative, with a VL domain of SEQ ID NO:01 and a VH domain of SEQ ID NO:02 (described in Dengl S, et al. Hapten-directed spontaneous disulfide shuffling: a universal technology for site directed covalent coupling of payloads to antibodies. FASEB J 2015; 29:1763-1779). The first precursor polypeptide comprised a light chain polypeptide of SEQ ID NO:03 (also referred to as “bio LC”), a first heavy chain polypeptide of SEQ ID NO:04 (also referred to as “bio HC”) and a second heavy chain polypeptide based on SEQ ID NO:05 (which represents the basic amino acid sequence without destabilizing mutation), with the destabilizing mutations as indicated below and a histidine tag. The second heavy chain polypeptide (also referred to as “dummy hole” polypeptide) comprised von N- to C-terminal direction a hinge region, a CH2 domain and a CH3 domain.

The second heterodimeric precursor polypeptide (also referred to as “anti-fluo precursor”) comprised a Fab fragment specifically binding to fluorescein (“fluo”) with a VL domain of SEQ ID NO:06 and a VH domain of SEQ ID NO:07. The second precursor polypeptide comprised a light chain polypeptide of SEQ ID NO:08 (also referred to as “fluo LC”), a first heavy chain polypeptide of SEQ ID NO:09 (also referred to as “fluo HC”) and a second heavy chain polypeptide based on SEQ ID NO:10 (which represents the basic amino acid sequence without destabilizing mutation) with the destabilizing mutations as indicated below and a histidine tag. The second heavy chain polypeptide (also referred to as “dummy knob” polypeptide) comprised von N- to C-terminal direction a hinge region, a CH2 domain and a CH3 domain.

The CH3 domains of the indicated polypeptide chains comprise the following mutations:

TABLE 1
Amino acid substitutions in CH3 domains of precursor polypeptides

| mutation | bio HC | dummy hole | fluo HC | dummy knob |
|---|---|---|---|---|
| knob/hole | T366W | T366S, | T366S, | T366W |
| | | L368A, | L368A, | |
| | | Y407W | Y407W | |
| destabilizing | no | yes | no | yes |
| mutation | | | | |
| cysteine | S354C | no | Y349C | no |
| mutation | | | | |

Anti-bio precursors were generated comprising dummy hole polypeptides having an amino acid sequence of SEQ ID NO:05, wherein one of the following amino acid substitutions was made: E357K, D356K, C349Y, C349A, C349W, E357F, A368F, F405W, V407Y, D399A F405W, L441Y, K409E, T394I, D356K E357K, L351Y, Q347K, S354V, K370E, S364L, K392E, K439E, or D399A S400K.

Anti-fluo precursors were generated comprising dummy knob polypeptides having an amino acid sequence of SEQ ID NO:10, wherein one of the following amino acid substitutions was made: K370E, K439E, C354S, C354S N297Q, S354E, S364L, Y407W, F405W, W366I K409E, K370E K439E, D399K K409E, Y349E, S364V, L368F, K392D, T394I, Q347K K360E, E357F, S400K, or L351F E357F.

Expression Plasmids for the Precursor Polypeptides were Generated as Follows: For the expression of anti-bio and anti-fluo precursors as reported herein a transcription unit comprising the following functional elements was used:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a nucleic acid encoding the respective precursor polypeptide, and
the bovine growth hormone polyadenylation sequence (BGH pA).
Beside the expression unit/cassette including the desired gene to be expressed the basic/standard mammalian expression plasmid contains
an origin of replication from the vector pUC18 which allows replication of this plasmid in E. coli, and
a beta-lactamase gene which confers ampicillin resistance in E. coli.

### Recombinant Production of Precursor Polypeptides

Transient expression of anti-bio and anti-fluo precursors as reported herein was performed in suspension-adapted Expi293F™ cells (A14527; Life Technologies™) in Expi293F™ Expression Medium (A1435101; Life Technologies™) with the transfection reagent mix ExpiFectamine™ 293 Transfection Kit (A14524; Life Technologies™).

Cells were passaged, by dilution, at least four times (volume 30 ml) after thawing in a 125 ml shake flask (Incubate/Shake at 37° C., 7% CO₂, 85% humidity, 135 rpm). The cells were expanded to 3×10⁵ cells/ml in 250 ml volume. Three days later, cells were split and newly seeded with a density of 1.3*10⁶ cells/ml in a 250 ml volume in a 1-liter shake flask. Transfection was performed 24 hours later at a cell density around 2.2-2.8×10⁶ cells/ml.

Before transfection 30 μg plasmid DNA were diluted in a final volume of 1.5 ml with pre-heated (water bath; 37° C.) Opti-MEM (Gibco). The solution was gently mixed and incubated at room temperature for not longer than 5 min. Then 1.5 ml of a pre-incubated solution of ExpiFectamine™ reagent in Opti-MEM was added to the DNA-OptiMEM solution. The resulting solution was gently mixed and incubated at room temperature for 20-30 minutes. The whole volume of mixture was added to a 100 ml shake flask, 50 ml falcon tube or deep-well in a 48 well deep-well plate with 30 ml Expi293F™ culture.

Transfected cells were incubated at 37° C., 7% CO₂, 85% humidity, for 7 days and shaken at 110 rpm for shake flasks and 205 rpm for falcon tubes.

16-24 h after transfection, 20 μl ExpiFectamine™ Enhancer 1 and 200 μl ExpiFectamine™ Enhancer 2 were added to the 30 ml cell culture.

The supernatant was harvested by centrifugation at 4,000 rpm, 4° C., for 20 minutes. Thereafter the cell-free-supernatant was filtered through a 0.22 μm bottle-top-filter and stored in a freezer (−20° C.).

The antibodies were purified from cell culture supernatants by affinity chromatography using MabSelectSure-Sepharose™ (GE Healthcare, Sweden).

Briefly, sterile filtered cell culture supernatants were captured on a MabSelectSuRe resin equilibrated with PBS buffer (10 mM Na₂HPO₄, 1 mM KH₂PO₄, 137 mM NaCl and 2.7 mM KCl, pH 7.4), washed with equilibration buffer and eluted with 25 mM sodium citrate at pH 3.0. The eluted fractions of the respective precursor polypeptides were pooled and neutralized with 2 M Tris, pH 9.0.

Alternatively, the precursor polypeptides were purified from cell culture supernatants by affinity chromatography using ani-Ckappa resin (KappaSelect, GE Healthcare, Sweden).

Briefly, sterile filtered cell culture supernatants were captured on a KappaSelect resin equilibrated with PBS buffer (10 mM Na2HPO4, 1 mM KH2PO4, 137 mM NaCl and 2.7 mM KCl, pH 7.4), washed with equilibration buffer and eluted with 25 mM sodium citrate at pH 3.0. The eluted precursor polypeptide fractions were pooled and neutralized with 2 M Tris, pH 9.0.

The identity of the precursor polypeptides was confirmed by mass spectrometry. For each individual sample, the conserved Fc N-glycosylation was removed enzymatically (using N-glycosidase F), the protein denatured (guanidine hydrochloride) and the disulfide bonds reduced (using DTT or TCEP). The samples were desalted by liquid chromatography (by size exclusion or reversed phase chromatography) and analyzed by mass spectrometry (Bruker Maxis Q-ToF). The identity of each molecule was confirmed by exact mass measurement and comparison with the theoretically expected molecule mass.

Analytical Size Exclusion Chromatography was carried out via a BioSuite High Resolution SEC Column (250 Å, Waters, USA) using a 200 mM K₂HPO₄/KH₂PO₄, 250 mM KCl, pH 7.0 running buffer at a flow rate of 1 mg/ml. Monomer content of all individual precursor polypeptides was assessed before reaction setup

TABLE 2
Monomer content of anti-bio precursor polypeptides with
a dummy hole chain having the indicated destabilizing
mutation in the CH3 domain (LMW . . . large molecular
weight side products; monomer = desired heterodimeric
precursor polypeptide; HMW . . . high molecular weight side products)

| | % SEC | % SEC | % SEC |
|---|---|---|---|
| destabilizing mutation | LMW | monomer | HMW |
| E357K | 12 | 88 | 0 |
| D356K | 23.5 | 73.3 | 3.3 |
| C349Y | 22.8 | 77.2 | 0 |
| C349A | 13.4 | 86.6 | 0 |
| C349W | 16.5 | 83.5 | 0 |
| E357F | 11.7 | 88.3 | 0 |
| A368F | 9.7 | 77.8 | 12.5 |
| F405W | 9 | 76.6 | 14.4 |
| V407Y | 13.4 | 68.7 | 17.9 |
| D399A F405W | 9.6 | 75.1 | 15.2 |
| L441Y | 12.3 | 72.9 | 14.8 |
| K409E | 8.1 | 73.5 | 18.3 |
| T394I | 19 | 62 | 19 |
| D356K E357K | 10.6 | 71.2 | 18.2 |
| L351Y | 8.5 | 74.4 | 17.1 |
| Q347K | 4.2 | 76.2 | 19.6 |
| S354V | 4.9 | 81.8 | 13.3 |
| K370E | 7 | 75.7 | 17.3 |
| S364L | 12.2 | 68.3 | 19.5 |
| K392E | 3.3 | 86.6 | 10.1 |
| K439E | 4.6 | 84.7 | 10.7 |
| D399A S400K | 12.4 | 78.2 | 9.4 |

TABLE 3
Monomer content of anti-fluo precursor polypeptides with a
dummy knob chain having the indicated destabilizing mutation in the
CH3 domain (LMW . . . large molecular weight side products; monomer = desired
heterodimeric precursor polypeptide; HMW . . . high molecular weight side products)

| | % SEC | % SEC | % SEC |
|---|---|---|---|
| destabilizing mutation | LMW | monomer | HMW |
| K370E | 6.6 | 90.5 | 2.9 |
| K439E | 2.6 | 95 | 2.4 |
| C354S | 7.9 | 84 | 8.1 |
| C354S N297Q | 7.2 | 76.6 | 16.2 |
| S354E | 7.9 | 83.3 | 8.8 |
| S364L | 7.8 | 83.4 | 8.8 |
| Y407W | 12.8 | 73.3 | 14 |
| F405W | 11.7 | 75.3 | 13 |
| W366I K409D | 16.7 | 77.5 | 5.8 |
| K370E K439E | 8.6 | 86 | 5.4 |
| D399K K409E | 3.6 | 94 | 2.4 |
| Y349E | 0 | 96.3 | 3.7 |
| S364V | 0 | 97.1 | 2.9 |
| L368F | 0.7 | 95.9 | 3.3 |
| K392D | 2.9 | 94.5 | 2.6 |
| T394I | 1.1 | 91.3 | 7.5 |
| Q347K K360E | 1.8 | 85.5 | 2.7 |
| E357F | 2.7 | 93 | 4.3 |
| S400K | 0 | 88.1 | 11.9 |
| L351F E357F | 3.9 | 83.8 | 12.4 |

### Example 2

### Analysis of Polypeptide Chain Exchange Efficiency by Direct Detection of Bispecific Product Polypeptide Formation by ELISA

To assess the impact of different destabilizing mutations on the polypeptide chain exchange, 460 exchange reactions were set up using the precursor polypeptides as generated in Example 1. The structure of the expected product polypeptides is depicted in FIG. 1. Presence of the bispecific anti-biocytinamid/anti-fluorescein product polypeptide was assessed by ELISA.

In order to start the exchange reaction anti-bio precursor polypeptides and anti-fluo precursor polypeptides were mixed in equimolar amounts (normalized to the % monomer SEC value to assure same amounts of intact molecules in single reactions) at a protein concentration of 2 μM in a total volume of 48 μl 1×PBS+0.05% Tween 20+0.25 mM TCEP on a 384 well REMP® plate (Brooks, #1800030). Of note, addition of the reducing agent TCEP reduces the hinge disulfides thus supporting dissociation of the polypeptide chains. After centrifugation, plates were sealed and incubated for one hour at 37° C. The resulting reacted mixture was analyzed via ELISA.

### A Biotin—Fluorescein Bridging ELISA was Subsequently Used to Quantify the Bispecific Antibody:

Therefore, white Nunc® MaxiSorp™ 384 well plates were coated with 1 μg/ml albumin-fluorescein isothiocyanate conjugate (Sigma, #A9771) and incubated overnight at 4° C. After washing 3 times with 90 μl PBST-buffer (PBST, double distilled water, 10×PBS+0.05% Tween 20) blocking buffer (1×PBS, 2% gelatin, 0.10% Tween-20) was added 90 μl/well and incubated for one hour at room temperature. After washing 3 times with 90 μl PBST-buffer, 25 μl of a 1:4 dilution of each reacted mixture was added to each well. After incubation for one hour at room temperature, plates were again washed 3 times with 90 μl PBST-buffer. 25 μl/well biotin-Cy5 conjugate in 0.5% BSA, 0.025% Tween-20, 1×PBS was added to a final concentration of 0.1 μg/ml and plates were incubated for one hour at room temperature. After washing 6 times with 90 μl PBST-buffer, 25 μl 1×PBS were added to each well. Cy5 fluorescence was measured at an emission wavelength of 670 nm (excitation at 649 nm) on a Tecan Safire 2 Reader.

A preformed anti-fluorescein/anti-biocytinamid bispecific reference antibody (bio light chain of SEQ ID NO:03, bio heavy chain of SEQ ID NO:04, fluo light chain of SEQ ID NO:08 and fluo heavy chain of SEQ ID NO:09) was used as a 100% control for the reaction outcome.

The preformed bispecific reference antibody was analyzed by analytical size exclusion chromatography as indicated above:

TABLE 4
Monomer content of bispecific reference antibody

| | % SEC | % SEC | % SEC |
|---|---|---|---|
| | LMW | monomer | HMW |
| anti-fluorescein/anti-biocytinamid | 5.8 | 94 | 0.2 |
| bispecific reference antibody | | | |

Absorbance signals from the reference antibody in the bridging ELISA setup was averaged from 23 reactions. This mean value was used as 100% bridging signal for normalization of all polypeptide chain exchange reactions. Assay variability of the reference antibody in the bridging ELISA is 100+/−15.2%. Polypeptide chain exchange reactions above 100% may lie within this variability. Additionally, potential aggregates that might occur in reaction mixtures might lead to increased bridging signals.

Results are indicated in Table 5. Relative absorbance values from the pairs of CH3 mutations considered to support polypeptide chain exchange are underlined.

TABLE 5
Formation of bispecific product polypeptide by polypeptide chain reaction
from anti-bio and anti-fluo precursor polypeptides comprising the indicated
destabilizing mutation(s) in the CH3 domain of the dummy chain. Columns indicate
destabilizing mutations in dummy hole polypeptide of the anti-bio precursor; lines
indicate destabilizing mutations in dummy knob polypeptide of anti-fluo precursor.
Relative absorbance as detected via bridging ELISA is indicated.

| | | | | | | | | | | D399A | |
|---|---|---|---|---|---|---|---|---|---|---|---|
| mutation | E357K | D356K | C349Y | C349A | C349W | E357F | A368F | F405W | V407Y | F405W | L441Y |
| K370E | 58 | 16 | 5 | 6 | 8 | 38 | 14 | 3 | 20 | 15 | 9 |
| K439E | 52 | 37 | 5 | 8 | 10 | 35 | 21 | 4 | 21 | 16 | 9 |
| C354S | 15 | 13 | 4 | 5 | 7 | 12 | 12 | 3 | 9 | 11 | 5 |
| C354S | 28 | 16 | 5 | 7 | 7 | 20 | 16 | 5 | 18 | 15 | 8 |
| N297Q | | | | | | | | | | | |
| S354E | 37 | 18 | 4 | 7 | 9 | 17 | 16 | 3 | 15 | 16 | 7 |
| S364L | 21 | 13 | 4 | 5 | 7 | 11 | 13 | 3 | 16 | 8 | 6 |
| Y407W | 135 | 33 | 6 | 7 | 13 | 63 | 23 | 4 | 90 | 24 | 8 |
| F405W | 93 | 34 | 9 | 9 | 13 | 38 | 25 | 4 | 64 | 21 | 11 |
| W366I | 130 | 36 | 8 | 10 | 13 | 61 | 45 | 4 | 101 | 43 | 8 |
| K409D | | | | | | | | | | | |
| K370E | 138 | 68 | 6 | 8 | 12 | 74 | 17 | 3 | 52 | 17 | 9 |
| K439E | | | | | | | | | | | |
| D399K | 144 | 34 | 6 | 8 | 10 | 62 | 32 | 4 | 87 | 29 | 9 |
| K409E | | | | | | | | | | | |
| Y349E | 175 | 38 | 7 | 9 | 15 | 109 | 36 | 7 | 73 | 26 | 14 |
| S364V | 13 | 7 | 4 | 5 | 6 | 15 | 17 | 4 | 13 | 10 | 7 |
| L368F | 34 | 17 | 5 | 8 | 11 | 45 | 39 | 5 | 31 | 19 | 13 |
| K392D | 44 | 13 | 4 | 7 | 9 | 54 | 27 | 4 | 60 | 41 | 12 |
| T394I | 82 | 29 | 10 | 16 | 16 | 81 | 64 | 9 | 100 | 22 | 21 |
| Q347K | 43 | 23 | 8 | 16 | 21 | 66 | 39 | 8 | 60 | 39 | 20 |
| K360E | | | | | | | | | | | |
| E357F | 11 | 7 | 5 | 7 | 7 | 13 | 16 | 6 | 10 | 9 | 7 |
| S400K | 25 | 20 | 8 | 11 | 13 | 32 | 25 | 7 | 25 | 18 | 12 |
| L351F | 24 | 12 | 6 | 9 | 8 | 22 | 11 | 7 | 17 | 12 | 9 |
| E357F | | | | | | | | | | | |
| | | | D356K | | | | | | | | D399A |
| mutation | K409E | T394I | E357K | L351Y | Q347K | S354V | K370E | S364L | K392E | K439E | S400K |
| K370E | 32 | 22 | 37 | 8 | 4 | 18 | 2 | 25 | 14 | 14 | 27 |
| K439E | 25 | 29 | 43 | 11 | 5 | 21 | 2 | 28 | 17 | 15 | 25 |
| C354S | 14 | 16 | 12 | 7 | 4 | 13 | 2 | 12 | 14 | 9 | 13 |
| C354S | 17 | 19 | 20 | 6 | 4 | 17 | 2 | 13 | 16 | 11 | 17 |
| N297Q | | | | | | | | | | | |
| S354E | 15 | 15 | 16 | 7 | 4 | 17 | 2 | 17 | 16 | 10 | 16 |
| S364L | 13 | 14 | 16 | 7 | 4 | 11 | 2 | 11 | 10 | 12 | 11 |
| Y407W | 96 | 46 | 93 | 10 | 4 | 34 | 2 | 82 | 32 | 18 | 44 |
| F405W | 62 | 25 | 60 | 13 | 5 | 24 | 2 | 54 | 33 | 18 | 33 |
| W366I | 89 | 44 | 93 | 11 | 5 | 23 | 2 | 80 | 28 | 18 | 74 |
| K409D | | | | | | | | | | | |
| K370E | 40 | 28 | 75 | 11 | 4 | 24 | 2 | 67 | 18 | 16 | 25 |
| K439E | | | | | | | | | | | |
| D399K | 95 | 55 | 84 | 8 | 4 | 23 | 2 | 91 | 31 | 15 | 54 |
| K409E | | | | | | | | | | | |
| Y349E | 90 | 50 | 88 | 11 | 4 | 48 | 3 | 80 | 25 | 13 | 29 |
| S364V | 15 | 16 | 15 | 6 | 4 | 14 | 2 | 15 | 7 | 8 | 7 |
| L368F | 54 | 35 | 41 | 11 | 6 | 34 | 2 | 37 | 20 | 13 | 13 |
| K392D | 77 | 45 | 81 | 10 | 4 | 30 | 2 | 64 | 26 | 11 | 55 |
| T394I | 114 | 51 | 106 | 20 | 9 | 41 | 4 | 79 | 45 | 30 | 61 |
| Q347K | 51 | 45 | 48 | 18 | 8 | 38 | 4 | 43 | 39 | 23 | 38 |
| K360E | | | | | | | | | | | |
| E357F | 11 | 11 | 11 | 7 | 6 | 9 | 4 | 11 | 8 | 9 | 9 |
| S400K | 22 | 36 | 25 | 11 | 7 | 24 | 5 | 32 | 38 | 19 | 24 |
| L351F | 18 | 15 | 27 | 10 | 6 | 18 | 4 | 36 | 16 | 18 | 15 |
| E357F | | | | | | | | | | | |

### Example 3

### Analysis of Polypeptide Chain Exchange Efficiency by Biochemical Quantification of Bispecific Product Formation

A subset of anti-bio and anti-fluo precursors were reacted to form 86 bispecific product polypeptides. For the reaction, equimolar amounts of precursor polypeptides as described in Example 1 were combined. Freshly prepared TCEP (60 eq. of 0.5 mM TCEP in 1×PBS pH 7.4, 0.05% Tween20) was added as a reducing agent to the reaction mixture and the mixture was incubated for 1 h at 37° C., mildly shaking at 300 rpm.

Bispecific product polypeptides were isolated as flow through from a cOmplete His-Tag column (Roche Diagnostics GmbH) equilibrated with 50 mM Na₂HPO₄, 300 mM NaCl, pH 8.0 and a flow of 1 ml/min. Remaining unreacted precursor polypeptides and product polypeptides consisting of dummy chain heterodimers were retained via their histidine-tag and eluted for analytical purposes with 50 mM Na₂HPO₄, 300 mM NaCl, 250 mM Imidazole at pH 8.0. Samples were concentrated via Amicon Ultra centrifugation tube (Millipore) to a protein concentration of 0.2-1.5 mg/ml and analyzed by size exclusion chromatography (SE-HPLC) with BioSuite High Resolution SEC Columns (250 Å, 5 μm, Waters, USA) using a 200 mM K₂HPO₄/KH₂PO₄, 250 mM KCl, pH 7.0 running buffer at a flow rate of 0.5 ml/min to determine the purity of the bispecific product formation.

CE-SDS (LabChip GXII (Perkin Elmer) was used to determine bispecific product polypeptide quality under non-reduced (re-formation of disulfide bridges) and reduced conditions (correct amount and presence of the protein chains). Samples were prepared for CE-SDS as follows: 5 μl, c=0.1-1 mg/ml were combined with 35 μl sample buffer or sample denaturating solution in a 96 well PCR plate, and incubated at 70° C. for 10 minutes while mildly shaking. For reducing conditions Reducing Agent (NuPage) was diluted 10 fold in HT Protein Express Sample Buffer (e.g. 100 μl Reducing agent+900 μl Sample Buffer). Subsequently 70 μl of pure water were added to the samples and the sample plate placed for analysis into the LAbChip System.

Results from 86 chain exchange reactions are shown in Table 6. Results from pairs of CH3 mutations with particularly high product yield are indicated in underlined and bold. Shown is the total yield of bispecific product polypeptide after polypeptide chain exchange and purification steps in mg. Product yield in % is calculated as the relative amount of the recombined product corrected to the maximum expected product mass. For this calculation, amounts of precursor polypeptides have been corrected to monomer-content as analyzed by analytical size exclusion chromatography, as only monomers are expected to be effective for recombination. Additionally, it was taken into account that the maximum expected product mass is limited by the less abundant precursor polypeptide.

TABLE 6
Yield of bispecific product polypeptide after polypeptide chain
exchange from anti-bio and anti-fluo precursor polypeptides comprising the indicated
destabilizing mutation(s) in the CH3 domain of the dummy chain and after purification.

| | | Product | Product |
|---|---|---|---|
| Destabilizing mutation | Destabilizing mutation | Yield | yield |
| in dummy hole CH3 | in dummy knob CH3 | [mg] | [%] |
| E357K | K370E | 0.42 | 55.17 |
| E357K | K370E | 0.49 | 67.12 |
| E357K | K439E | 0.26 | 36.35 |
| D356K | K370E | 0.21 | 33.62 |
| D356K | K439E | 0.10 | 16.32 |
| D356K | C354S N297Q | 0.15 | 25.45 |
| D356K | K370E K439E | 0.19 | 45.49 |
| D356K | L368F | 0.14 | 24.35 |
| D356K | T394I | 0.11 | 19.05 |
| D356K | Q347K K360E | 0.13 | 22.47 |
| D356K | V397Y | 0.08 | 19.68 |
| D356K | S400K | 0.13 | 41.63 |
| C349Y | K370E | 0.01 | 5.49 |
| C349Y | C354S N297Q | 0.00 | 0.56 |
| C349Y | S364L | 0.00 | 0.12 |
| C349Y | K370E K439E | 0.00 | 0.86 |
| C349Y | D399K K409E | 0.00 | 0.31 |
| C349Y | L368F | 0.00 | 1.63 |
| C349Y | T394I | 0.01 | 2.79 |
| C349Y | V397Y | 0.00 | 0.41 |
| A368F | K370E | 0.14 | 27.51 |
| A368F | K439E | 0.17 | 32.89 |
| A368F | C354S N297Q | 0.13 | 25.08 |
| A368F | S364L | 0.06 | 11.67 |
| A368F | K370E K439E | 0.08 | 18.25 |
| A368F | D399K K409E | 0.14 | 27.25 |
| A368F | L368F | 0.13 | 23.76 |
| A368F | K392D | 0.10 | 18.84 |
| A368F | T394I | 0.13 | 24.85 |
| A368F | Q347K K360E | 0.13 | 25.56 |
| A368F | E357F | 0.06 | 10.45 |
| A368F | V397Y | 0.12 | 26.88 |
| A368F | S400K | 0.07 | 13.14 |
| V407Y | K370E | 0.30 | 89.70 |
| V407Y | C354S N297Q | 0.11 | 33.47 |
| V407Y | S364L | 0.10 | 30.56 |
| V407Y | K370E K439E | 0.28 | 82.55 |
| V407Y | Q347K K360E | 0.17 | 49.93 |
| V407Y | V397Y | 0.31 | 90.59 |
| D399A F405W | K370E | 0.24 | 32.37 |
| D399A F405W | K439E | 0.24 | 33.33 |
| D399A F405W | C354S N297Q | 0.13 | 20.27 |
| D399A F405W | S364L | 0.09 | 14.54 |
| D399A F405W | K370E K439E | 0.16 | 36.75 |
| D399A F405W | Q347K K360E | 0.28 | 43.69 |
| D399A F405W | E357F | 0.05 | 7.29 |
| D399A F405W | V397Y | 0.13 | 29.49 |
| D399A F405W | S400K | 0.12 | 21.48 |
| T394I | C354S N297Q | 0.09 | 14.63 |
| T394I | S364L | 0.08 | 12.90 |
| T394I | W366I K409D | 0.16 | 32.81 |
| T394I | D399K K409E | 0.25 | 48.24 |
| T394I | L368F | 0.16 | 25.63 |
| T394I | K392D | 0.16 | 25.41 |
| T394I | T394I | 0.14 | 22.56 |
| T394I | E357F | 0.04 | 7.17 |
| T394I | S400K | 0.09 | 14.98 |
| S354V | K370E | 0.12 | 19.63 |
| S354V | K439E | 0.15 | 23.60 |
| S354V | C354S N297Q | 0.04 | 6.80 |
| S354V | W366I K409D | 0.01 | 1.90 |
| S354V | K370E K439E | 0.05 | 10.61 |
| S354V | D399K K409E | 0.11 | 22.31 |
| S354V | T394I | 0.06 | 9.34 |
| S354V | Q347K K360E | 0.04 | 6.71 |
| S354V | V397Y | 0.07 | 16.24 |
| S354V | S400K | 0.05 | 8.31 |
| S364L | K439E | 0.43 | 86.59 |
| S364L | C354S N297Q | 0.15 | 30.85 |
| S364L | W366I K409D | 0.35 | 70.95 |
| S364L | K370E K439E | 0.41 | 92.99 |
| S364L | D399K K409E | 0.37 | 75.71 |
| S364L | L368F | 0.22 | 43.66 |
| S364L | K392D | 0.25 | 49.62 |
| S364L | T394I | 0.29 | 58.08 |
| S364L | Q347K K360E | 0.22 | 43.51 |
| S364L | S400K | 0.12 | 23.78 |
| K439E | C354S N297 | 0.13 | 38.34 |
| K439E | W366I K409D | 0.14 | 41.17 |
| K439E | D399K K409E | 0.15 | 42.63 |
| K439E | L368F | 0.12 | 35.36 |
| K439E | T394I | 0.12 | 33.48 |
| D399A S400K | K370E | 0.03 | 4.38 |
| D399A S400K | K439E | 0.05 | 8.15 |
| D399A S400K | K370E K439E | 0.09 | 19.41 |
| D399A S400K | Q347K K360E | 0.05 | 7.58 |
| D399A S400K | V397Y | 0.06 | 14.16 |

### Example 4

### Generation of Monospecific Precursor Polypeptides for the Generation of Activatable Binding Sites Upon Polypeptide Chain Exchange

For assessing formation of bispecific anti-LeY/anti-CD3 antibodies from monospecific precursor polypeptides, monospecific precursor polypeptides of a domain arrangement as depicted for the first and second heterodimeric precursor polypeptides indicated in FIG. 2 and FIG. 3 were generated.

### Precursor Polypeptides Devoid of CH2 Domain

In a first set of experiments, heterodimeric precursor polypeptides with a domain arrangement as depicted in FIG. 2 were provided. The precursor polypeptides are devoid of CH2 domains and comprise an antibody variable domain arranged at the N-terminal end of the CH3 domains.

In a first alternative, the following precursor polypeptides were provided:
A first heterodimeric precursor polypeptide (also referred to as “anti-LeY-CD3(VH)-knob precursor”) comprised a Fab fragment specifically binding to LeY. The anti-LeY-CD3(VH)-knob precursor comprised a light chain polypeptide of SEQ ID NO:11 (also referred to as “LeY LC”), a first heavy chain polypeptide of SEQ ID NO:12 (also referred to as “LeY-CD3(VH)-knob HC”) comprising a VH domain derived from an antibody specifically binding to CD3 (“CD3(VH)”) and a second heavy chain polypeptide based on SEQ ID NO:13 (which represents the basic amino acid sequence without destabilizing mutation), with the destabilizing mutations as indicated below and a histidine tag. The second heavy chain polypeptide (also referred to as “dummy-VL-hole” polypeptide) comprised von N- to C-terminal direction a hinge region, a VL domain derived from an antibody specifically binding to digoxigenin (“dig”) and a CH3 domain.
A second heterodimeric precursor polypeptide (also referred to as “anti-LeY-CD3(VL)-hole precursor”) comprised a Fab fragment specifically binding to LeY. The anti-LeY-CD3(VL)-hole precursor comprised the light chain polypeptide of SEQ ID NO:11, i.e. the LeY LC; a first heavy chain polypeptide of SEQ ID NO:14 (also referred to as “LeY-CD3(VL)-hole HC”) comprising a VL domain derived from an antibody specifically binding to CD3 (“CD3(VL)”) and a second heavy chain polypeptide based on SEQ ID NO: 15 (which represents the basic amino acid sequence without destabilizing mutation) with the destabilizing mutations as indicated below and a histidine tag. The second heavy chain polypeptide (also referred to as “dummy-VH-knob” polypeptide) comprised von N- to C-terminal direction a hinge region, a VH domain derived from a non-binding antibody and a CH3 domain.

In a second alternative, the following precursor polypeptides were provided:
A first heterodimeric precursor polypeptide (also referred to as “anti-LeY-CD3(VL)-knob precursor”) comprised a Fab fragment specifically binding to LeY. The anti-LeY-CD3(VL)-knob precursor comprised the light chain polypeptide of SEQ ID NO:11, i.e. the LeY LC, a first heavy chain polypeptide of SEQ ID NO:16 (also referred to as “LeY-CD3(VL)-knob HC”) comprising the CD3(VL) domain and a second heavy chain polypeptide based on SEQ ID NO:17 (which represents the basic amino acid sequence without destabilizing mutation), with the destabilizing mutations as indicated below and a histidine tag. The second heavy chain polypeptide (also referred to as “dummy-VH-hole” polypeptide) comprised von N- to C-terminal direction a hinge region, a VH domain derived from a non-binding antibody and a CH3 domain.
A second heterodimeric precursor polypeptide (also referred to as “anti-LeY-CD3(VH)-hole precursor”) comprised a Fab fragment specifically binding to LeY. The anti-LeY-CD3(VH)-hole precursor comprised the light chain polypeptide of SEQ ID NO:11, i.e. the LeY LC; a first heavy chain polypeptide of SEQ ID NO: 18 (also referred to as “LeY-CD3(VH)-hole HC”) comprising the CD3(VH) domain and a second heavy chain polypeptide based on SEQ ID NO:19 (which represents the basic amino acid sequence without destabilizing mutation) with the destabilizing mutations as indicated below and a histidine tag. The second heavy chain polypeptide (also referred to as “dummy-VL-knob” polypeptide) comprised von N- to C-terminal direction a hinge region, a VL domain derived from an anti-dig antibody and a CH3 domain.

The indicated polypeptide chains comprise the following mutations:

TABLE 7
Amino acid substitutions in CH3 domains of precursor polypeptides

| | LeY- | | LeY- | |
|---|---|---|---|---|
| | CD3(VH)- | dummy- | CD3(VL)- | dummy- |
| mutation | knob HC | VL-hole | hole HC | VH-knob |
| knob/hole | T366W | T366S, | T366S, | T366W |
| | | L368A, | L368A, | |
| | | Y407W | Y407W | |
| destabilizing | no | yes | no | yes |
| mutation | | | | |
| cysteine | no | no | no | no |
| mutation | | | | |
| | LeY- | | LeY- | |
| | CD3(VL)- | dummy- | CD3(VH)- | dummy- |
| mutation | knob HC | VH-hole | hole HC | VL-knob |
| knob/hole | T366W | T366S, | T366S, | T366W |
| | | L368A, | L368A, | |
| | | Y407W | Y407W | |
| destabilizing | no | yes | no | yes |
| mutation | | | | |
| cysteine | no | no | no | no |
| mutation | | | | |

Precursor Polypeptides with Fc Domain

In a second set of experiments, heterodimeric precursor polypeptides with a domain arrangement as depicted in FIG. 3 were provided. The precursor polypeptides comprise a full Fc domain and comprise an antibody variable domain arranged at the N-terminal end of the CH2 domains.

In a first alternative, the following precursor polypeptides were provided:
A first heterodimeric precursor polypeptide (also referred to as “anti-LeY-CD3(VH)-Fc(knob) precursor”) comprised a Fab fragment specifically binding to LeY. The anti-LeY-CD3(VH)-Fc(knob) precursor comprised the light chain polypeptide of SEQ ID NO:11, i.e. the LeY LC, a first heavy chain polypeptide of SEQ ID NO:20 (also referred to as “LeY-CD3(VH)-Fc(knob) HC”) comprising the CD3(VH) domain and a second heavy chain polypeptide based on SEQ ID NO:21 (which represents the basic amino acid sequence without destabilizing mutation), with the destabilizing mutations as indicated below and a histidine tag. The second heavy chain polypeptide (also referred to as “dummy-VL-Fc(hole)” polypeptide) comprised von N- to C-terminal direction a hinge region, a VL domain derived from an antibody specifically binding to digoxigenin (“dig”), a CH2 domain and a CH3 domain.
A second heterodimeric precursor polypeptide (also referred to as “anti-LeY-CD3(VL)-Fc(hole) precursor”) comprised a Fab fragment specifically binding to LeY. The anti-LeY-CD3(VL)-Fc(hole) precursor comprised the LeY LC; a first heavy chain polypeptide of SEQ ID NO:22 (also referred to as “LeY-CD3(VL)-Fc(hole) HC”) comprising the CD3(VL) domain and a second heavy chain polypeptide based on SEQ ID NO:23 (which represents the basic amino acid sequence without destabilizing mutation) with the destabilizing mutations as indicated below and a histidine tag. The second heavy chain polypeptide (also referred to as “dummy-VH-Fc(knob)” polypeptide) comprised von N- to C-terminal direction a hinge region, a VH domain derived from an anti-dig antibody, a CH2 domain and a CH3 domain.

In a second alternative, the following precursor polypeptides were provided:
A first heterodimeric precursor polypeptide (also referred to as “anti-LeY-CD3(VL)-Fc(knob) precursor”) comprised a Fab fragment specifically binding to LeY. The anti-LeY-CD3(VL)-Fc(knob) precursor comprised the LeY LC, a first heavy chain polypeptide of SEQ ID NO:24 (also referred to as “LeY-CD3(VL)-Fc(knob) HC”) comprising the CD3(VL) domain and a second heavy chain polypeptide based on SEQ ID NO:25 (which represents the basic amino acid sequence without destabilizing mutation), with the destabilizing mutations as indicated below and a histidine tag. The second heavy chain polypeptide (also referred to as “dummy-VH-Fc(hole)” polypeptide) comprised von N- to C-terminal direction a hinge region, a VH domain derived from an anti-dig antibody, a CH2 domain and a CH3 domain.
A second heterodimeric precursor polypeptide (also referred to as “anti-LeY-CD3(VH)-Fc(hole) precursor”) comprised a Fab fragment specifically binding to LeY. The anti-LeY-CD3(VH)-Fc(hole) precursor comprised the LeY LC; a first heavy chain polypeptide of SEQ ID NO:26 (also referred to as “LeY-CD3(VH)-Fc(hole) H) comprising the CD3(VH) domain and a second heavy chain polypeptide based on SEQ ID NO:27 (which represents the basic amino acid sequence without destabilizing mutation) with the destabilizing mutations as indicated below and a histidine tag. The second heavy chain polypeptide (also referred to as “dummy-VL-Fc(knob)” polypeptide) comprised von N- to C-terminal direction a hinge region, a VL domain derived from an anti-dig antibody, a CH2 domain and a CH3 domain.

The indicated polypeptide chains comprise the following mutations:

TABLE 8
Amino acid substitutions in CH3 domains of precursor polypeptides

| | LeY- | | | |
|---|---|---|---|---|
| | CD3(VH)- | dummy- | LeY- | dummy- |
| | Fc(knob) | VL- | CD3(VL)- | VH- |
| mutation | HC | Fc(hole) | Fc(hole) HC | Fc(knob) |
| knob/hole | T366W | T366S, | T366S, | T366W |
| | | L368A, | L368A, | |
| | | Y407W | Y407W | |
| destabilizing | no | yes | no | yes |
| mutation | | | | |
| cysteine | S354C | no | Y349C | no |
| mutation | | | | |
| | LeY- | | | |
| | CD3(VL)- | dummy- | LeY- | dummy- |
| | Fc(knob) | VH- | CD3(VH)- | VL- |
| mutation | HC | Fc(hole) | Fc(hole) HC | Fc(knob) |
| knob/hole | T366W | T366S, | T366S, | T366W |
| | | L368A, | L368A, | |
| | | Y407W | Y407W | |
| destabilizing | no | yes | no | yes |
| mutation | | | | |
| cysteine | S354C | no | Y349C | no |
| mutation | | | | |

Heterodimeric precursor polypeptides were generated comprising dummy VL-hole polypeptides of SEQ ID NO:13 and dummy VH-hole polypeptides of SEQ ID NO:17 as indicated above having the amino acid sequence of the respective dummy polypeptide as indicated above, wherein one of the following amino acid substitutions was made: E357K, A368F, D399A F405W, S364L, Y407W, or S354V.

Heterodimeric precursor polypeptides were generated comprising dummy VH-knob polypeptides of SEQ ID NO:15 and dummy VL-knob polypeptides of SEQ ID NO:19 as indicated above having the amino acid sequence of the respective dummy polypeptide as indicated above, wherein one of the following amino acid substitutions was made: K370E, no destabilizing mutation, W366I K409D, V397Y, or K392D.

Heterodimeric precursor polypeptides were generated comprising dummy VL-Fc(hole) polypeptides of SEQ ID NO:21 and dummy-VH-Fc(hole) polypeptides of SEQ ID NO:25 as indicated above having the amino acid sequence of the respective dummy polypeptide as indicated above, wherein one of the following amino acid substitutions was made: E357K, A368F, D399A F405W, S364L, D356K, or S354V.

Heterodimeric precursor polypeptides were generated comprising dummy-VH-Fc(knob) polypeptides of SEQ ID NO:23 and dummy-VL-Fc(knob) polypeptides of SEQ ID NO:27 as indicated above having the amino acid sequence of the respective dummy polypeptide as indicated above, wherein one of the following amino acid substitutions was made: K370E, no destabilizing mutation, W366I K409D, V397Y, K392D, or K370E K439E.

Recombinant Production of Precursor Polypeptides

Expression was done by co-transfection of plasmids the three polypeptide chains of each precursor polypeptide into mammalian cells (e.g. HEK293 or Expi293F™) by state of the art technologies.

For the expression of precursor polypeptides indicated above a transcription unit comprising the following functional elements was used:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a nucleic acid encoding the respective precursor polypeptide, and
a 3′-non-translated region with a polyadenylation signal sequence.

Beside the expression unit/cassette including the desired gene to be expressed the basic/standard mammalian expression plasmid contains
an origin of replication, which allows replication of this plasmid in E. coli, and
a beta-lactamase gene which confers ampicillin resistance in E. coli.

The expression cassettes encoding comprising the precursor polypeptide chains were generated by PCR and/or gene synthesis and assembled by known recombinant methods and techniques by connection of the according nucleic acid segments e.g. using unique restriction sites in the respective plasmids. The subcloned nucleic acid sequences were verified by DNA sequencing. For transient transfections larger quantities of the plasmids were prepared by plasmid preparation from transformed E. coli cultures (HiSpeed Plasmid Maxi Kit, Qiagen).

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

The precursor polypeptide derivatives were generated by transient transfection with the respective plasmid using the HEK293-F system (Invitrogen) or the Expi293F™ system (Live Technologies) according to the manufacturer's instruction. Briefly, HEK293-F cells (Invitrogen) or Expi293F™ cells (Live Technologies) growing in suspension either in a shake flask or in a stirred fermenter in serum-free FreeStyle™ 293 expression medium (Invitrogen) or Expi293F™ Expression Medium (Life Technologies) were transfected with the respective expression plasmid and 293Fectin™, fectin (Invitrogen) or PEIpro (Polyplus) or the reagent mix ExpiFectamine™ 293 Transfection Kit (Life Technologies). For 1-2 L shake flasks (Corning), HEK293-F cells or Expi293F™ cells were seeded at a density of 1-1.3*10⁶ cells/mL in 250-600 mL and incubated at 120 rpm, 8% CO₂. The day after the cells were transfected with the appropriate expression plasmids. HEK293-F cells were transfected at a cell density of approx. 1.5*10⁶ cells/mL with ca. 42 mL mix of A) 20 mL Opti-MEM (Invitrogen) with 300 μg total plasmid DNA (0.5 μg/mL) and B) 20 ml Opti-MEM+1.2 mL 293 fectin or fectin (2 μL/mL) or 750 μl PEIpro (1.25 μL/mL). Expi293F™ cells were transfected at a cell density around 2.2-2.8×10⁶ cells/ml. Before transfection 30 μg plasmid DNA were diluted in a final volume of 1.5 ml with pre-heated (water bath; 37° C.) Opti-MEM (Gibco). The solution was gently mixed and incubated at room temperature for not longer than 5 min. Then 1.5 ml of a pre-incubated solution of ExpiFectamine™ reagent in Opti-MEM was added to the DNA-OptiMEM solution. The resulting solution was gently mixed and incubated at room temperature for 20-30 minutes. The whole volume of mixture was added to a 100 ml shake flask with 30 ml Expi293F™ culture. The cultures were incubated at 37° C., 7% CO₂, 85% humidity, for 7 days at 110 rpm. For the Expi293F™ cultures, 20 μl ExpiFectamine™ Enhancer 1 and 200 μl ExpiFectamine™ Enhancer 2 were added to 30 ml cell culture 15-24 h after transfection. According to the glucose consumption glucose solution was added during the course of the fermentation. Correctly assembled split cytokine molecules were secreted into culture supernatants like standard IgGs. The supernatant containing the split cytokine molecules was harvested after 5-10 days and split cytokine molecules were either directly purified from the supernatant or the supernatant was frozen at −20° C. and stored.

Precursor polypeptides having a full Fc-region (CH2-CH3) bind to ProteinA. These precursors were purified by proteinA chromatography followed by SEC.

Precursor polypeptides devoid of CH2 domains but contained kappa light chains. Therefore, these precursors were purified by applying standard kappa light chain affinity chromatography. The precursor polypeptides were purified from cell culture supernatants by affinity chromatography using KappaSelect (GE Healthcare, Sweden) and Superdex 200 size exclusion (GE Healthcare, Sweden) chromatography or ion exchange chromatography.

Briefly, sterile filtered cell culture supernatants were captured on a KappaSelect resin equilibrated with PBS buffer (10 mM Na2HPO4, 1 mM KH₂PO₄, 137 mM NaCl and 2.7 mM KCl, pH 7.4), washed with equilibration buffer and eluted with 50 mM sodium citrate, 150 mM NaCl at pH 3.0. The eluted precursor polypeptide fractions were pooled and neutralized with 2M Tris, pH 9.0. The precursor polypeptide pools were further purified by size exclusion chromatography or ion exchange chromatography. For size exclusion chromatography a Superdex™ 200 μg HiLoad™ 16/600 (GE Healthcare, Sweden) column equilibrated with 20 mM histidine, 140 mM NaCl, pH 6.0. For ion exchange chromatography, the protein sample obtained from KappaSelect purification was diluted 1:10 in 20 mM histidine, pH 6.0 and loaded on a HiTrap™ SP HP ion exchange (GE Healthcare, Sweden) column equilibrated with buffer A (20 mM histidine, pH 6.0). A gradient of 0-100% buffer B (20 mM histidine, 1 M NaCl, pH 6.0) was applied to elute different protein species.

Purity and integrity were analyzed after purification by SDS-PAGE. Protein solution (13 μl) was mixed with 5 μl 4×NuPAGE LDS sample buffer (Invitrogen) and 2 μl 10×NuPAGE sample reducing agent (Invitrogen) and heated to 95° C. for 5 min. Samples were loaded on a NuPAGE 4-12% Bis-Tris gel (Invitrogen) and run according to the manufacturer's instructions using a Novex Mini-Cell (Invitrogen) and NuPAGE MES SDS running buffer (Life Technologies). Gels were stained using InstantBlue™ Coomassie protein stain. Furthermore, integrity and uniformity of proteins was analyzed using analytical size exclusion chromatography.

(CE-)SDS-PAGE revealed that all expected polypeptide chains were present in the preparations; analytical size exclusion confirmed >90% purity of the preparations. For review of methods for assessment of antibody purity, see, e.g., Flatman, S. et al., J. Chrom. B 848 (2007) 79-87.

### Example 5

### Determination of Polypeptide Chain Exchange Via T Cell Activation Assay

To assess the impact of different destabilizing mutations on the polypeptide chain exchange, exchange reactions were set up using the precursor polypeptides as generated in Example 4. The structure of the expected product polypeptides is depicted in FIG. 2 for the precursor polypeptides devoid of a CH2 domain and in FIG. 3 for the precursor polypeptides comprising a full Fc domain. Polypeptide chain exchange results in formation of an antigen binding site specifically binding to CD3. Presence of the bispecific anti-LeY/anti-CD3 product polypeptide was assessed by cell-based assay.

The influence of different CH3 interface mutations on the efficacy of this chain exchange reaction was evaluated in a cell based reporter assay system composed of LeY-expressing MCF7 cells and a Jurkat reporter cell line (Promega J1621) according to the following principle: Binding of the first and second heterodimeric polypeptides to MCF7 cells and polypeptide chain exchange results in formation of an antigen binding site specifically binding to CD3. Jurkat cells expressing CD3 are bound by the antigen binding site specifically binding to CD3, which results in luciferase expression from the Jurkat cells. Luminescence was detected after addition of BioGlo substrate.

In brief, the cell based assay was carried out as follows in a 384-well plate. RPMI1640 with 10% FCS was used as assay media. 6×10⁴ Jurkat effector cells were mixed with 2×10⁴ MCF7 cells in total volume of 10 μl. Precursor polypeptides were applied either alone or in combination at 200 nM and 2 nM to end up in a final volume of 30 μl. Cells were incubated for 20 hours at cell culture conditions. 24 μl of Bioglo were added to each well, incubated for 5 minutes. Luminescence was measured in an Infinite® 200 PRO reader (TECAN).

TABLE 9
Formation of bispecific product polypeptide by polypeptide chain reaction
from precursor polypeptides devoid of CH2 domains as defined above in Example 4
comprising the indicated destabilizing mutation(s) in the CH3 domain of the dummy
chain. Results are shown from the exchange reaction at precursor polypeptide concentration
of 200 nM. Luminescence efficacy is rated as follows: <10% . . . “−”, 10-29% . . . “+”,
30-50% . . . “++”, >50% . . . “+++”)

| | mutation | K370E | no mutation | W366I K409D | V397Y | K392D |
|---|---|---|---|---|---|---|
| | | dummy-VL-knob | | | | |
| dummy-VH-hole | E357K | +++ | + | +++ | ++ | ++ |
| | A368F | − | − | + | − | − |
| | D399A | ++ | − | ++ | ++ | ++ |
| | F405W | | | | | |
| | S364L | +++ | + | +++ | ++ | ++ |
| | Y407W | +++ | ++ | +++ | ++ | |
| | S354V | + | − | + | − | − |
| | | dummy-VH-knob | | | | |
| dummy-VL-hole | E357K | ++ | + | +++ | +++ | ++ |
| | A368F | − | − | + | + | − |
| | D399A | + | + | ++ | ++ | ++ |
| | F405W | | | | | |
| | S364L | ++ | ++ | +++ | +++ | ++ |
| | Y407W | ++ | ++ | +++ | +++ | ++ |
| | S354V | + | + | ++ | ++ | + |

TABLE 10
Formation of bispecific product polypeptide by polypeptide chain reaction
from precursor polypeptides devoid of CH2 domains as defined above in Example 4
comprising the indicated destabilizing mutation(s) in the CH3 domain of the dummy
chain. Results are shown from the exchange reaction at precursor polypeptide concentration
of 2 nM. Luminescence efficacy is rated as follows: <2% . . . “−”, 2-4% . . . “+”,
5-10% . . . “++”, >10% . . . “+++”)

| | mutation | K370E | no mutation | W366I K409D | V397Y | K392D |
|---|---|---|---|---|---|---|
| | | dummy-VL-knob | | | | |
| dummy-VH-hole | E357K | + | − | ++ | − | − |
| | A368F | − | − | − | − | − |
| | D399A | − | − | + | − | − |
| | F405W | | | | | |
| | S364L | + | − | ++ | − | − |
| | Y407W | + | − | ++ | − | − |
| | S354V | − | − | − | − | − |
| | | dummy-VH-knob | | | | |
| dummy-VL-hole | E357K | − | − | +++ | ++ | − |
| | A368F | − | − | − | − | − |
| | D399A | − | − | + | + | − |
| | F405W | | | | | |
| | S364L | − | − | ++ | ++ | − |
| | Y407W | − | − | +++ | +++ | − |
| | S354V | − | − | − | − | − |

TABLE 11
Formation of bispecific product polypeptide by polypeptide chain reaction
from precursor polypeptides having an Fc domain as defined above in Example 4
comprising the indicated destabilizing mutation(s) in the CH3 domain of the dummy
chain. Results are shown from the exchange reaction at precursor polypeptide
concentration of 2 nM. Luminescence efficacy is rated as follows: <10% . . . “−”,
10-19% . . . “+”, 20-50% . . . “++”, >50% . . . “+++”

| | | dummy-VH-Fc(knob) | | | | | |
|---|---|---|---|---|---|---|---|
| | | | no | W366I | | | K370E |
| | mutation | K370E | mut. | K409D | V397Y | K392D | K439E |
| dummy-VL-Fc(hole) | E357K | +++ | + | +++ | +++ | +++ | +++ |
| | A368F | ++ | − | ++ | ++ | ++ | ++ |
| | D399A | + | − | ++ | + | + | + |
| | F405W | | | | | | |
| | S364L | +++ | + | +++ | +++ | +++ | +++ |
| | D356K | + | − | + | + | + | + |
| | S354V | + | − | + | + | + | + |

### Example 6

### Generation of Further Monospecific Precursor Polypeptides Comprising a Full Fc Domain

For assessing formation of bispecific anti-biocytinamid/anti-fluorescein antibodies from monospecific precursor polypeptides, monospecific precursor polypeptides of a domain arrangement as depicted for the first and second heterodimeric precursor polypeptides indicated in FIG. 1 were generated. Note that in this experiments the knobs and holes mutations were arranged on the opposite chains.

The first heterodimeric precursor polypeptide (also referred to as “anti-fluo precursor”) comprised a Fab fragment specifically binding to fluorescein (“fluo”), a biotin derivative, with a VL domain of SEQ ID NO:06 and a VH domain of SEQ ID NO:07. The first precursor polypeptide comprised a light chain polypeptide of SEQ ID NO:08 (also referred to as “fluo LC”), a first heavy chain polypeptide of SEQ ID NO:29 (also referred to as “fluo HC”) and a second heavy chain polypeptide based on SEQ ID NO:05 (which represents the basic amino acid sequence without destabilizing mutation), with the destabilizing mutations as indicated below and a C-tag. The second heavy chain polypeptide (also referred to as “dummy hole” polypeptide) comprised von N- to C-terminal direction a hinge region, a CH2 domain and a CH3 domain.

The second heterodimeric precursor polypeptide (also referred to as “anti-bio precursor”) comprised a Fab fragment specifically binding to biocytinamid (“bio”) with a VL domain of SEQ ID NO:01 and a VH domain of SEQ ID NO:02. The second precursor polypeptide comprised a light chain polypeptide of SEQ ID NO:03 (also referred to as “bio LC”), a first heavy chain polypeptide of SEQ ID NO:28 (also referred to as “bio HC”) and a second heavy chain polypeptide based on SEQ ID NO:10 (which represents the basic amino acid sequence without destabilizing mutation) with the destabilizing mutations as indicated below and a C-tag. The second heavy chain polypeptide (also referred to as “dummy knob” polypeptide) comprised von N- to C-terminal direction a hinge region, a CH2 domain and a CH3 domain.

First and second heterodimeric precursor polypeptides were generated according to the methods as disclosed in Example 1.

The CH3 domains of the indicated polypeptide chains comprise the following mutations:

TABLE 12
Purification yield and monomer content of anti-fluo precursor polypeptides
with a dummy hole chain having the indicated destabilizing
mutation in the CH3 domain (purification yield [mg/ml] = amount
of purified antibody per liter expression volume, corrected by %
monomer peak; monomer = desired heterodimeric precursor polypeptide)

| destabilizing | purification | % SEC |
|---|---|---|
| mutation | yield [mg/L] | monomer |
| E357F | 45.6 | 96.6 |
| V407Y | 30.1 | 95.7 |
| D356K | 39.5 | 90.7 |
| K409E | 78.3 | 99.1 |
| D356K-E357K | 42.1 | 95.9 |
| S364L | 65.9 | 95.8 |
| S364A | 75.0 | 81.5 |
| S364I | 61.9 | 90.8 |
| S364Q | 34.3 | 98.5 |
| E357F-T394I | 43.2 | 98.9 |
| E357F-S364L | 56.1 | 81.2 |
| D356K-V407Y | 28.7 | 84.7 |
| D356K-S364L | 43.4 | 92.0 |
| E357K-T394I | 40.6 | 98.0 |

TABLE 13
Purification yield and monomer content of anti-bio precursor polypeptides
with a dummy knob chain having the indicated destabilizing
mutation in the CH3 domain (purification yield [mg/ml] = amount
of purified antibody per liter expression volume, corrected by %
monomer peak; monomer = desired heterodimeric precursor polypeptide)

| | purification | % SEC |
|---|---|---|
| destabilizing mutation | yield [mg/L] | monomer |
| Y407W | 104.1 | 95.3 |
| W366I-K409D | 40.2 | 91.0 |
| D399K-K409E | 88.0 | 91.3 |
| K370E-K439E | 45.7 | 98.9 |
| Y349E | 153.5 | 94.8 |
| T394I | 49.7 | 98.5 |
| Y349D | 96.2 | 98.2 |
| Y349F | 76.8 | 91.4 |
| Y349H | 75.5 | 89.9 |
| Y349E-Y407W | 75.8 | 94.6 |
| W366I-F405W-K409D | 106.8 | 93.7 |
| T394I-F405W | 54.5 | 90.8 |
| W366I-K409D-K439E | 37.9 | 93.4 |
| Y349E-K439E | 72.0 | 98.0 |

### Example 7

Analysis of Polypeptide Chain Exchange Efficiency of Precursor Polypeptides from Example 6

To assess the impact of different destabilizing mutations on the polypeptide chain exchange, exchange reactions between the precursor polypeptides generated in Example 6 were performed. The experiment was carried out according to the methodology described in Example 2. The structure of the expected product polypeptides is depicted in FIG. 1.

TABLE 14
Formation of bispecific product polypeptide by polypeptide chain
exchange reaction from anti-bio and anti-fluo precursor polypeptides comprising the
indicated destabilizing mutation(s) in the CH3 domain of the dummy chain. Columns
indicate destabilizing mutations in dummy hole polypeptide of the anti-fluo
precursor; lines indicate destabilizing mutations in dummy knob polypeptide of anti-
bio precursor. Values indicate the exchange efficiency via product yield [%]. The
experimentally obtained yield is related to the maximum possible yield of bispecific
antibody. The maximum possible yield of bispecific antibody is corrected by the
lowest % monomer peak SEC of the two respective input formats in each reaction, as
only monomers are expected to be effective for recombination.

| | | | | | D356K | | |
|---|---|---|---|---|---|---|---|
| mutation | E357F | V407Y | D356K | K409E | E357K | S364L | S364A |
| Y407W | 63.5 | 79.5 | 43.1 | 74.6 | 73.8 | 74.7 | 55.3 |
| W366I | 65.9 | 85.4 | 40.2 | 88.7 | 79.8 | 86.8 | 50.4 |
| K409D | | | | | | | |
| D399K | 78.5 | 92.8 | 45.1 | 73.6 | 77.3 | 81.7 | 73.0 |
| K409E | | | | | | | |
| K370E | 73.9 | 70.6 | 68.1 | 55.8 | 82.2 | 69.3 | 71.4 |
| K439E | | | | | | | |
| Y349E | 75.4 | 75.4 | 52.3 | 72.6 | 79.3 | 85.0 | 68.9 |
| T394I | 51.9 | 70.8 | 39.1 | 57.6 | 61.3 | 58.3 | 43.7 |
| Y349D | 71.4 | 77.0 | 51.0 | 67.2 | 79.0 | 76.9 | 66.9 |
| Y349F | 56.8 | 59.8 | 43.2 | 43.9 | 56.1 | 56.3 | 49.1 |
| Y349H | 64.5 | 70.3 | 47.1 | 57.1 | 77.0 | 63.3 | 56.2 |
| Y349E | 72.2 | 82.0 | 49.6 | 79.9 | 84.8 | 84.0 | 67.1 |
| Y407W | | | | | | | |
| Y366I | 81.4 | 76.2 | 43.0 | 73.6 | 78.4 | 77.5 | 69.7 |
| F405W | | | | | | | |
| K409D | | | | | | | |
| T394I | 68.7 | 84.3 | 48.9 | 67.6 | 68.0 | 73.5 | 53.5 |
| F405W | | | | | | | |
| W366I | 74.3 | 89.2 | 54.7 | 75.7 | 77.0 | 77.7 | 60.3 |
| K409D | | | | | | | |
| K439E | | | | | | | |
| Y349E | 75.2 | 84.1 | 63.0 | 68.7 | 70.4 | 71.4 | 70.0 |
| K439E | | | | | | | |
| Y349E | 79.8 | 80.7 | 46.1 | 74.7 | 75.8 | 82.2 | 63.1 |
| W366I | | | | | | | |
| K409D | | | | | | | |
| | | | E357F | E357F | D356K | D356K | E357K |
| mutation | S364I | S364Q | T394I | S364L | V407Y | S364L | T394I |
| Y407W | 81.4 | 70.2 | 87.4 | 90.6 | 79.3 | 78.4 | 66.3 |
| W366I | 79.6 | 77.5 | 81.0 | 86.6 | 96.4 | 85.2 | 94.9 |
| K409D | | | | | | | |
| D399K | 77.0 | 80.7 | 82.0 | 94.5 | 95.1 | 79.2 | 91.9 |
| K409E | | | | | | | |
| K370E | 87.9 | 71.7 | 72.6 | 90.6 | 95.6 | 87.9 | 76.8 |
| K439E | | | | | | | |
| Y349E | 79.3 | 73.0 | 83.0 | 84.7 | 85.0 | 82.1 | 70.6 |
| T394I | 64.8 | 52.8 | 74.8 | 77.6 | 78.2 | 69.0 | 57.5 |
| Y349D | 86.8 | 77.8 | 72.3 | 88.7 | 89.6 | 88.7 | 75.9 |
| Y349F | 58.0 | 51.3 | 61.5 | 73.3 | 60.8 | 56.7 | 63.5 |
| Y349H | 76.9 | 65.9 | 73.7 | 77.2 | 75.3 | 77.1 | 80.6 |
| Y349E | 86.3 | 79.2 | 86.8 | 99.4 | 84.8 | 86.6 | 82.7 |
| Y407W | | | | | | | |
| W366I | 78.2 | 76.2 | 79.8 | 100 | 81.4 | 79.1 | 81.9 |
| F405W | | | | | | | |
| K409D | | | | | | | |
| T394I | 84.2 | 66.6 | 77.8 | 87.9 | 96.7 | 71.2 | 79.3 |
| F405W | | | | | | | |
| W366I | 73.3 | 72.8 | 78.0 | 89.4 | 96.2 | 78.3 | 81.4 |
| K409D | | | | | | | |
| K439E | | | | | | | |
| Y349E | 91.5 | 69.1 | 78.3 | 95.4 | 97.7 | 76.6 | 75.8 |
| K439E | | | | | | | |
| Y349E | 84.7 | 76.0 | 83.8 | 98.4 | 90.1 | 76.6 | 75.8 |
| W366I | | | | | | | |
| K409D | | | | | | | |

### Example 8

### Generation of Further Monospecific Precursor Polypeptides Comprising a Full Fc Domain, Wherein the CH3 Domains of the Precursor Polypeptides Comprise Knobs-into-Hole Mutations but not Comprise Cysteine Mutations

For assessing formation of bispecific anti-biocytinamid/anti-fluorescein antibodies from monospecific precursor polypeptides, monospecific precursor polypeptides of a domain arrangement as depicted for the first and second heterodimeric precursor polypeptides indicated in FIG. 1 were generated. Note that in this experiments the knobs and holes mutations were arranged on the opposite chains.

First and second heterodimeric precursor polypeptides as described in Example 6 were generated according to the structure and methods as disclosed therein.

Furthermore, deviating from Example 1, the bio HC is based on SEQ ID NO: 28 however with a serine residue at position 354 and the fluo HC is based in SEQ ID NO: 29 however with a tyrosine residue at position 349. The mutations of the CH3 domains are therefore summarized as follows:

TABLE 15
Amino acid substitutions in CH3 domains of precursor polypeptides

| mutation | bio HC | dummy knob | fluo HC | dummy hole |
|---|---|---|---|---|
| knob / hole | T366S, | T366W | T366W | T366S, L368A, |
| | L368A, | | | Y407W |
| | Y407W | | | |
| destabilizing | no | yes | no | yes |
| mutation | | | | |
| cysteine | no | no | no | no |
| mutation | | | | |

The CH3 domains of the indicated polypeptide chains comprise the following mutations:

TABLE 16
Purification yield and monomer content of anti-bio
precursor polypeptides with a dummy
knob chain having the indicated destabilizing
mutation in the CH3 domain (purification
yield [mg/ml] = amount of purified antibody per liter
expression volume, corrected by % monomer peak;
monomer = desired heterodimeric precursor polypeptide)

| destabilizing | purification | % SEC |
|---|---|---|
| mutation | yield [mg/L] | monomer |
| E357F | 29.6 | 98.0 |
| V407Y | 16.8 | 97.4 |
| D356K | 22.9 | 91.2 |
| K409E | 27.0 | 96.7 |
| D356K-E357K | 68.4 | 99.0 |
| S364L | 23.4 | 94.9 |
| S364A | 38.3 | 95.1 |
| S364I | 69.5 | 97.9 |
| S364Q | 36.4 | 95.3 |
| E357F-T394I | 15.8 | 98.8 |
| E357F-S364L | 16.9 | 94.5 |
| D356K-V407Y | 16.7 | 93.7 |
| D356K-S364L | 30.2 | 98.2 |
| E357K-T394I | 27.9 | 96.4 |

TABLE 17
Purification yield and monomer content of anti-fluo
precursor polypeptides with a dummy hole chain having
the indicated destabilizing mutation in the CH3 domain
(purification yield [mg/ml] = amount of purified antibody
per liter expression volume, corrected by % monomer
peak; monomer = desired heterodimeric precursor polypeptide)

| | purification | % SEC |
|---|---|---|
| destabilizing mutation | yield [mg/L] | monomer |
| Y407W | 60.6 | 94.3 |
| W366I-K409D | 59.7 | 98.3 |
| K370E-K439E | 59.0 | 98.3 |
| D399K-K409E | 40.4 | 85.5 |
| Y349E | 39.8 | 99.7 |
| T394I | 8.6 | 95.7 |
| Y349D | 71.1 | 96.6 |
| Y349F | 30.2 | 98.2 |
| Y349H | 33.9 | 95.1 |
| Y349E-Y407W | 50.6 | 99.0 |
| W366I-F405W-K409D | 87.4 | 98.3 |
| T394I-F405W | 63.8 | 96.6 |
| W366I-K409D-K439E | 60.2 | 97.7 |
| Y349E-W366I-K409D | 58.1 | 98.4 |
| Y349E-K439E | 71.2 | 97.0 |

### Example 9

Analysis of Polypeptide Chain Exchange Efficiency of Precursor Polypeptides from Example 8

To assess the impact of different destabilizing mutations on the polypeptide chain exchange, exchange reactions between the precursor polypeptides generated in Example 8 were performed. The experiment was carried out according to the methodology described in Example 2.

TABLE 18
Formation of bispecific product polypeptide by polypeptide chain
exchange reaction from anti-bio and anti-fluo precursor polypeptides comprising the
indicated destabilizing mutation(s) in the CH3 domain of the dummy chain. Columns
indicate destabilizing mutations in dummy hole polypeptide of the anti-fluo
precursor; lines indicate destabilizing mutations in dummy knob polypeptide of anti-
bio precursor. Values indicate the exchange efficiency via product yield [%]. The
experimentally obtained yield is related to the maximum possible yield of bispecific
antibody. The maximum possible yield of bispecific antibody is corrected by the
lowest % monomer peak SEC of the two respective input formats in each reaction, as
only monomers are expected to be effective for recombination.

| | | | | | D356K | | |
|---|---|---|---|---|---|---|---|
| mutation | E357F | V407Y | D356K | K409E | E357K | S364L | S364A |
| Y407W | 51.5 | 67.2 | 26.4 | 56.3 | 72.9 | 66.5 | 23.6 |
| W366I K409D | 58.5 | 80.3 | 31.3 | 72.5 | 82.5 | 80.3 | 8.8 |
| D399K K409E | 97.4 | 100.1 | 48.5 | 90.8 | 91.6 | 86.2 | 24.2 |
| K370E K439E | 58.6 | 71.5 | 54.3 | 47.0 | 78.4 | 82.9 | 25.4 |
| Y349E | 67.3 | 77.2 | 38.1 | 50.0 | 80.0 | 77.8 | 24.7 |
| T394I | 41.2 | 78.8 | 26.8 | 50.6 | 60.5 | 53.9 | 15.7 |
| Y349D | 63.3 | 76.3 | 38.6 | 52.1 | 78.7 | 80.2 | 32.6 |
| Y349F | 29.5 | 55.5 | 25.6 | 23.9 | 39.4 | 36.1 | 10.6 |
| Y349H | 45.0 | 52.1 | 25.9 | 40.0 | 62.6 | 54.3 | 10.8 |
| Y349E Y407W | 71.4 | 81.8 | 44.1 | 72.8 | 75.2 | 83.8 | 21.6 |
| W366I F405W | 86.4 | 84.8 | 43.4 | 79.2 | 80.3 | 83.2 | 23.9 |
| K409D | | | | | | | |
| T394I F405W | 69.4 | 77.6 | 35.9 | 69.9 | 69.6 | 85.7 | 24.5 |
| W366I K409D | 67.8 | 86.7 | 59.0 | 73.7 | 80.3 | 78.0 | 16.3 |
| K439E | | | | | | | |
| Y349E K439E | 52.6 | 60.3 | 39.2 | 50.1 | 48.4 | 62.5 | 24.1 |
| Y349E W366I | 74.0 | 75.2 | 41.7 | 73.0 | 75.2 | 81.4 | 20.2 |
| K409D | | | | | | | |
| | | | E357F | E357F | D356K | D356K | E357K |
| mutation | S364I | S364Q | T394I | S364L | V407Y | S364L | T394I |
| Y407W | 27.8 | 60.2 | 82.7 | 64.8 | 69.6 | 72.9 | 81.6 |
| W366I K409D | 65.6 | 67.1 | 76.0 | 62.2 | 88.1 | 84.6 | 89.6 |
| D399K K409E | 50.9 | 92.7 | 88.0 | 78.3 | 99.7 | 86.6 | 83.0 |
| K370E K439E | 68.4 | 64.8 | 76.4 | 71.3 | 85.8 | 83.2 | 69.3 |
| Y349E | 65.2 | 71.3 | 81.9 | 66.6 | 80.6 | 74.7 | 65.7 |
| T394I | 51.7 | 43.2 | 68.0 | 55.9 | 70.7 | 64.2 | 69.7 |
| Y349D | 77.2 | 62.8 | 82.7 | 69.7 | 78.5 | 77.7 | 64.1 |
| Y349F | 28.5 | 29.2 | 47.8 | 42.7 | 52.8 | 39.8 | 54.7 |
| Y349H | 33.8 | 50.0 | 69.0 | 50.8 | 56.1 | 55.6 | 72.0 |
| Y349E Y407W | 78.0 | 77.0 | 75.7 | 76.0 | 89.8 | 80.9 | 88.7 |
| W366I F405W | 64.2 | 72.3 | 72.9 | 73.5 | 79.0 | 78.1 | 77.6 |
| K409D | | | | | | | |
| T394I F405W | 77.9 | 74.5 | 72.3 | 68.4 | 92.3 | 85.8 | 76.7 |
| W366I K409D | 69.5 | 76.9 | 74.8 | 69.5 | 89.6 | 76.2 | 83.0 |
| K439E | | | | | | | |
| Y349E K439E | 14.7 | 60.3 | 55.3 | 55.3 | 65.5 | 64.1 | 64.8 |
| Y349E W366I | 80.0 | 82.1 | 74.3 | 80.2 | 83.9 | 71.0 | 77.3 |
| K409D | | | | | | | |

Results demonstrate that polypeptide chain exchange is detectable for heterodimeric precursor polypeptides, wherein the knobs-into-holes mutations are not stabilized by additional cysteine mutations.

### Example 10

Generation of Further Monospecific Precursor Polypeptides Comprising a Full Fc Domain, with Different Mutations in the CH3 Domains of the Precursor Polypeptides

For assessing formation of bispecific anti-biocytinamid/anti-fluorescein antibodies from monospecific precursor polypeptides, monospecific precursor polypeptides of a domain arrangement as depicted for the first and second heterodimeric precursor polypeptides indicated in FIG. 1 were generated. Note that in this experiments the knobs and holes mutations were arranged on the opposite chains.

First and second heterodimeric precursor polypeptides as described in Example 6 were generated according to the structure and methods as disclosed therein, however with the following difference:

Deviating from Example 6, three precursor polypeptides specifically binding to fluorescein were generated, wherein the fluo HC is based on SEQ ID NO: 29 and dummy-hole polypeptide is based on SEQ ID NO: 05 with the following CH3 mutations:

| | | |
|---|---|---|
| mutation | fluo HC | dummy hole |

Precursor polypeptide #01

| | | |
|---|---|---|
| knob/hole | T366W | T366S, L368A, Y407W |
| destabilizing | No | E357K |
| mutation | | |
| cysteine | S354C | no |
| mutation | | |

Precursor polypeptide #02

| | | |
|---|---|---|
| knob/hole | T366W | T366S, L368A, Y407W |
| destabilizing | no | E357K |
| mutation | | |
| cysteine | no | Y349C |
| mutation | | |

Precursor polypeptide #03

| | | |
|---|---|---|
| knob/hole | T366W | T366S, L368A, Y407W |
| destabilizing | no | E357K |
| mutation | | |
| cysteine | S354C | Y349C |
| mutation | | |

Deviating from Example 1, three precursor polypeptides specifically binding to biocytinaminde were generated, wherein the bio HC is based on SEQ ID NO: 28 and dummy-knob polypeptide is based on SEQ ID NO: 10 with the following CH3 mutations:

| | | |
|---|---|---|
| mutation | bio HC | dummy knob |

Precursor polypeptide #04

| | | |
|---|---|---|
| knob/hole | T366S, | T366W |
| | L368A, | |
| | Y407W | |
| destabilizing | no | K370E |
| mutation | | |
| cysteine | Y349C | no |
| mutation | | |

Precursor polypeptide #05

| | | |
|---|---|---|
| knob/hole | T366S, | T366W |
| | L368A, | |
| | Y407W | |
| destabilizing | no | K370E |
| mutation | | |
| cysteine | no | S354C |
| mutation | | |

Precursor polypeptide #06

| | | |
|---|---|---|
| knob/hole | T366S, | T366W |
| | L368A, | |
| | Y407W | |
| destabilizing | no | K370E |
| mutation | | |
| cysteine | Y349C | S354C |
| mutation | | |

TABLE 19
Purification yield and monomer content of
indicated precursor polypeptides (purification yield
[mg/ml] = amount of purified antibody per liter
expression volume, corrected by % monomer peak;
monomer = desired heterodimeric precursor polypeptide)

| Mutations | purification | % SEC |
|---|---|---|
| dummy | yield [mg/L] | monomer |
| #01 | 70.0 | 97.4 |
| #02 | 17.1 | 92.1 |
| #03 | 67.4 | 99.6 |
| #04 | 114.3 | 96.1 |
| #05 | 44.7 | 96.1 |
| #06 | 142.6 | 98.8 |

### Example 11

Analysis of Polypeptide Chain Exchange Efficiency of Precursor Polypeptides from Example 10

To assess the impact of different destabilizing mutations on the polypeptide chain exchange, exchange reactions between the precursor polypeptides generated in Example 10 were performed. The experiment was carried out according to the methodology described in Example 2.

TABLE 20
Formation of bispecific product polypeptide by polypeptide
chain exchange reaction from indicated anti-bio and anti-fluo
precursor polypeptides. Values indicate the exchange
efficiency via product yield [%]. The experimentally obtained
yield is related to the maximum possible yield of bispecific
antibody. The maximum possible yield of bispecific
antibody is corrected by the lowest % monomer peak
SEC of the two respective input formats in each reaction,
as only monomers are expected to be effective for recombination.

| #Precursor | | | |
|---|---|---|---|
| polypeptide | #01 | #02 | #03 |
| #04 | 59.6 | 67.4 | <10 |
| #05 | 39.5 | 43.8 | <10 |
| #06 | <10 | 10.5 | <10 |

Results indicate that the polypeptide chain occurs independent of arranging cysteine mutations either on the dummy chain polypeptide or on the polypeptide chain comprising an antigen binding moiety.

1. A set of heterodimeric precursor polypeptides comprising:
a) a first heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation,
wherein the first heterodimeric precursor polypeptide comprises a first antigen binding moiety, wherein at least a part of the first antigen binding moiety is arranged on one of the two polypeptide chains comprising the CH3 domain, and
b) a second heterodimeric precursor polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation,
wherein the second heterodimeric precursor polypeptide comprises a second antigen binding moiety, wherein at least a part of the second antigen binding moiety is arranged on one of the two polypeptide chains comprising the CH3 domain;
wherein
A) either i) within the first heterodimeric precursor polypeptide the polypeptide chain comprising the CH3 domain comprising the knob mutation comprises at least a part of the first antigen binding moiety and within the second heterodimeric precursor polypeptide the polypeptide chain comprising the CH3 domain with the hole mutation comprises at least a part of the second antigen binding moiety, or ii) within the first heterodimeric precursor polypeptide the polypeptide chain comprising the CH3 domain comprising the hole mutation comprises at least a part of the first antigen binding moiety and within the second heterodimeric precursor polypeptide the polypeptide chain comprising the CH3 domain with the knob mutation comprises at least a part of the second antigen binding moiety; and wherein
B) either
i) the CH3 domain of the first heterodimeric precursor polypeptide comprising the knob mutation and the CH3 domain of the second heterodimeric precursor polypeptide comprising the hole mutation, or
ii) the CH3 domain of the first heterodimeric precursor polypeptide comprising the hole mutation and the CH3 domain of the second heterodimeric precursor polypeptide comprising the knob mutation comprise the following amino acid substitutions, wherein the numbering is according to the Kabat numbering system:
the CH3 domain with the hole mutation comprises at least one amino acid substitution selected from the group of
replacement of S354 with a hydrophobic amino acid;
replacement of D356 with a positively charged amino acid;
replacement of E357 with a positively charged amino acid or with a hydrophobic amino acid;
replacement of D356 with a positively charged amino acid, and replacement of E357 with a positively charged amino acid or with a hydrophobic amino acid;
replacement of S364 with a hydrophobic amino acid;
replacement of A368 with a hydrophobic amino acid;
replacement of E392 with a negatively charged amino acid;
replacement of T394 with a hydrophobic amino acid;
replacement of D399 with a hydrophobic amino acid and replacement of S400 with a positively charged amino acid;
replacement of D399 with a hydrophobic amino acid and replacement of F405 with a positively charged amino acid;
replacement of V407 with a hydrophobic amino acid; and
replacement of K409 with a negatively charged amino acid; and
replacement of K439 with a negatively charged amino acid;
the CH3 domain with the knob mutation comprises at least one amino acid substitution selected from the group of:
replacement of Q347 with a positively charged amino acid, and replacement of K360 with a negatively charged amino acid;
replacement of Y349 with a negatively charged amino acid;
replacement of L351 with a hydrophobic amino acid, and replacement of E357 with a hydrophobic amino acid;
replacement of S364 with a hydrophobic amino acid;
replacement of W366 with a hydrophobic amino acid, and replacement of K409 with a negatively charged amino acid;
replacement of L368 with a hydrophobic amino acid;
replacement of K370 with a negatively charged amino acid;
replacement of K370 with a negatively charged amino acid, and replacement of K439 with a negatively charged amino acid;
replacement of K392 with a negatively charged amino acid;
replacement of T394 with a hydrophobic amino acid;
replacement of V397 with a hydrophobic amino acid;
replacement of D399 with a positively charged amino acid, and replacement of K409 with a negatively charged amino acid;
replacement of S400 with a positively charged amino acid;
F405W;
Y407W; and
replacement of K439 with a negatively charged amino acid.

2. The set of heterodimeric polypeptides according to claim 1 or 2, wherein the CH3 domains indicated in b) comprise the following amino acid substitutions:
the CH3 domain with the hole mutation comprises at least one amino acid substitution selected from the group of S354V, D356K, E357K, E357F, S364L, A368F, K392E, T394I, V407Y, K409E, K439E and a double mutation D399A S400K; and
the CH3 domain with the knob mutation comprises at least one amino acid substitution selected from the group of Y349E, S364V, L368F, K370E, K392D, T394I, V397Y, S400K, F405W, Y407W, K349E, and double mutations Q347K K360E, L351F E357F, W366I K409E, and D399K K409E.

3. The set of heterodimeric polypeptides according to one of claims 1 to 4, wherein the CH3 domains indicated in b) comprise the following amino acid substitutions:
the CH3 domain with the hole mutation comprises at least one amino acid substitution selected from the group of D356K, E357K, E357F, S364L, V407Y, K409E, and a double mutation D399A S400K; and
the CH3 domain with the knob mutation comprises at least one amino acid substitution selected from the group of Y349E, K370E, K392D, T394I, V397Y, F405W, Y407W, K349E, and double mutations Q347K K360E, W366I K409E, and D399K K409E.

4. The set of heterodimeric polypeptides according to one of the preceding claims, wherein in case the CH3 domain with the knob mutation indicated in b) comprises a mutation E357K, the CH3 domain with the hole mutation indicated in b) does not comprise a mutation K370E; and wherein in case the CH3 domain with the knob mutation indicated in b) comprises a mutation D356K, the CH3 domain with the hole mutation indicated in b) does not comprise a mutation K439E.

5. The set of heterodimeric polypeptides according to one of the preceding claims, wherein either i) the CH3 domain comprising the knob mutation of the first heterodimeric precursor polypeptide comprises a cysteine mutation and the CH3 domain comprising the hole mutation of the second heterodimeric precursor polypeptide comprises a cysteine mutation, or ii) the CH3 domain comprising the hole mutation of the first heterodimeric precursor polypeptide comprises a cysteine mutation and the CH3 domain comprising the knob mutation of the second heterodimeric precursor polypeptide comprises a cysteine mutation.

6. The set of heterodimeric polypeptides according to claim 5, wherein within the first heterodimeric precursor polypeptide the CH3 domain comprising the knob mutation comprises a substitution S354C and the CH3 domain comprising the hole mutation comprises Y at position 349; and wherein within the second heterodimeric precursor polypeptide the CH3 domain comprising the hole mutation comprises a substitution Y349C and the CH3 domain comprising the knob mutation comprises S at position 354.

7. The set of heterodimeric polypeptides according to one of the preceding claims, wherein the first antigen binding moiety and/or the second antigen binding moiety is an antibody fragment.

8. The set of heterodimeric polypeptides according to one of the preceding claims, wherein
a) the first heterodimeric precursor polypeptide comprises:
a first heavy chain polypeptide comprising a CH3 domain and a first antibody variable domain,
a second heavy chain polypeptide comprising a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
a light chain polypeptide comprising a second antibody variable domain, wherein the first and second antibody variable domain together form a first antigen binding site specifically binding to a target antigen; and wherein
b) the second heterodimeric precursor polypeptide comprises:
a third heavy chain polypeptide comprising a CH3 domain and a third antibody variable domain,
a fourth heavy chain polypeptide comprising a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
a light chain polypeptide comprising a fourth antibody variable domain, wherein the third and fourth antibody variable domain together form a second antigen binding site specifically binding to a target antigen; and wherein
c) either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation.

9. The set of heterodimeric polypeptides according to one of the preceding claims, wherein the first heterodimeric precursor polypeptide and the second heterodimeric precursor polypeptide comprise at least two polypeptide chains comprising from N- to C-terminal direction a hinge region, a CH2 domain and the CH3 domain.

10. The set of heterodimeric polypeptides according to claim 9, wherein the first heterodimeric precursor polypeptide and the second heterodimeric precursor polypeptide comprise an interchain disulfide bond in the hinge region.

11. The set of heterodimeric polypeptides according to one of the preceding claims, wherein the first heterodimeric precursor polypeptide comprises one polypeptide chain comprising a VL domain and the CH3 domain, and wherein the second heterodimeric precursor polypeptide comprises one polypeptide chain comprising a VH domain and the CH3 domain, wherein said VL domain and said VH domain specifically bind to an antigen when associated to a pair of a VH domain and a VL domain.

12. A heterodimeric polypeptide comprising at least two polypeptide chains comprising a CH3 domain, wherein the two polypeptide chains comprising the CH3 domain are associated with each other via the CH3 domains and form a heterodimer, wherein one of the CH3 domains comprises a knob mutation and the other CH3 domain comprises a hole mutation; wherein the heterodimeric polypeptide comprises a first antigen binding moiety, wherein at least a part of the first antigen binding moiety is arranged on one of the two polypeptide chains comprising the CH3 domain; and wherein the heterodimeric polypeptide comprises a second antigen binding moiety, wherein at least a part of the second antigen binding moiety is arranged on the other one of the two polypeptide chains comprising the CH3 domain; and
wherein the CH3 domain with the hole mutation comprises at least one amino acid substitution selected from the group of:
replacement of S354 with a hydrophobic amino acid;
replacement of D356 with a positively charged amino acid;
replacement of E357 with a positively charged amino acid or with a hydrophobic amino acid;
replacement of D356 with a positively charged amino acid, and replacement of E357 with a positively charged amino acid or with a hydrophobic amino acid;
replacement of S364 with a hydrophobic amino acid;
replacement of A368 with a hydrophobic amino acid;
replacement of E392 with a negatively charged amino acid;
replacement of T394 with a hydrophobic amino acid;
replacement of D399 with a hydrophobic amino acid and replacement of S400 with a positively charged amino acid;
replacement of D399 with a hydrophobic amino acid and replacement of F405 with a positively charged amino acid;
replacement of V407 with a hydrophobic amino acid; and
replacement of K409 with a negatively charged amino acid; and
replacement of K439 with a negatively charged amino acid; and wherein
the CH3 domain with the knob mutation comprises at least one amino acid substitution selected from the group of:
replacement of Q347 with a positively charged amino acid, and replacement of K360 with a negatively charged amino acid;
replacement of Y349 with a negatively charged amino acid;
replacement of L351 with a hydrophobic amino acid, and replacement of E357 with a hydrophobic amino acid;
replacement of S364 with a hydrophobic amino acid;
replacement of W366 with a hydrophobic amino acid, and replacement of K409 with a negatively charged amino acid;
replacement of L368 with a hydrophobic amino acid;
replacement of K370 with a negatively charged amino acid;
replacement of K370 with a negatively charged amino acid, and replacement of K439 with a negatively charged amino acid;
replacement of K392 with a negatively charged amino acid;
replacement of T394 with a hydrophobic amino acid;
replacement of V397 with a hydrophobic amino acid;
replacement of D399 with a positively charged amino acid, and replacement of K409 with a negatively charged amino acid;
replacement of S400 with a positively charged amino acid;
F405W;
Y407W; and
replacement of K439 with a negatively charged amino acid.

13. A method for generating a heterodimeric polypeptide comprising the steps of:
a) contacting a first heterodimeric precursor polypeptide and a second heterodimeric precursor polypeptide, as defined in one of claims 1 to 12 to form a third heterodimeric polypeptide comprising at least one polypeptide chain comprising a CH3 domain from the first heterodimeric precursor polypeptide and at least one polypeptide chain comprising a CH3 domain from the second heterodimeric polypeptide, and
b) recovering the third heterodimeric polypeptide.

14. The method according to claim 13, wherein
i) within the first heterodimeric precursor polypeptide the polypeptide chain comprising the CH3 domain with the knob mutation and within the second heterodimeric precursor polypeptide the polypeptide chain comprising the CH3 domain with the hole mutation; or
ii) within the first heterodimeric precursor polypeptide the polypeptide chain comprising the CH3 domain with the hole mutation and within the second heterodimeric precursor polypeptide the polypeptide chain comprising the CH3 domain with the knob mutation
comprise a tagging moiety, and wherein the method comprises the step of recovering the third heterodimeric polypeptide via a tag-specific affinity chromatography.

15. A heterodimeric polypeptide obtained by a method according to any one of claims 13 or 14.

16. A method for identifying a multispecific heterodimeric polypeptide comprising the steps of
a) generating a plurality of multispecific heterodimeric polypeptides by subjecting each combination of
a first heterodimeric precursor polypeptide from a plurality of first heterodimeric precursor polypeptides comprising an antigen binding moiety specifically binding to a first antigen, and
a second heterodimeric precursor polypeptide from a plurality of second heterodimeric precursor polypeptides comprising an antigen binding moiety specifically binding to a second antigen,
to a method according to one of claims 13 to 15; and
b) individually detecting a desired characteristic of each multispecific heterodimeric polypeptide from the plurality of multispecific heterodimeric polypeptide generated in step a), and
c) selecting a multispecific heterodimeric polypeptide.

17. The method according to claim 37, wherein the desired characteristic is selected from affinity and thermostability.

18. A multispecific heterodimeric polypeptide obtained by a method according to any one of claims 16 or 17.

19. A first heterodimeric precursor polypeptide as defined in any one of claims 1 to 12.

20. A second heterodimeric precursor polypeptide as defined in any one of claims 1 to 12.


SEQUENCE LISTING (29 sequences)

SEQ ID NO: 1 - protein, 112 aa, Artificial Sequence
  anti-biocytinamid VL
DIVMTQTPLSLSVTPGQPASISCKSSQSLVHSNGNTYLRWYLQKPGQSPKVLIYKVSNRVSGVPDRFSGSGSGTDFTLKISRVEAEDVGVYYCSQSTHVPWTFGQGTKLEIK

SEQ ID NO: 2 - protein, 118 aa, Artificial Sequence
  anti-biocytinamid VH
GVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSS

SEQ ID NO: 3 - protein, 219 aa, Artificial Sequence
  anti-biocytinamid light chain polypeptide
DIVMTQTPLSLSVTPGQPASISCKSSQSLVHSNGNTYLRWYLQKPGQSPKVLIYKVSNRVSGVPDRFSGSGSGTDFTLKISRVEAEDVGVYYCSQSTHVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 448 aa, Artificial Sequence
  anti-biocytinamid heavy chain polypeptide with knob
GVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 239 aa, Artificial Sequence
  heavy chain hinge-CH2-CH3 polypeptide with hole and tag
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSHHHHHHHH

SEQ ID NO: 6 - protein, 107 aa, Artificial Sequence
  anti-fluorescein VL
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIK

SEQ ID NO: 7 - protein, 118 aa, Artificial Sequence; identical to SEQ ID NO: 2
  anti-fluorescein VH

SEQ ID NO: 8 - protein, 214 aa, Artificial Sequence
  anti-fluorescein light chain polypeptide
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 448 aa, Artificial Sequence; identical to SEQ ID NO: 4
  anti-fluorescein heavy chain polypeptide with hole

SEQ ID NO: 10 - protein, 239 aa, Artificial Sequence
  heavy chain hinge-CH2-CH3 polypeptide with knob and tag
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSHHHHHHHH

SEQ ID NO: 11 - protein, 219 aa, Artificial Sequence
  anti-LeY light chain polypeptide
DVLMTQSPLSLPVSLGDQASISCRSSQIIVHSNGNTYLEWYLQKPGQSPKLLIYKVSNRFSGVPDRFSGSGSGTDFTLKISRVEAEDLGVYYCFQGSHVPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 468 aa, Artificial Sequence
  anti-LeY heavy chain polypeptide with knob and VH anti-CD3
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSEVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 227 aa, Artificial Sequence
  heavy chain polypeptide with hole and VL and tag
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSHHHHHHHH

SEQ ID NO: 14 - protein, 461 aa, Artificial Sequence
  anti-LeY heavy chain polypeptide with hole and VL anti-CD3
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - protein, 240 aa, Artificial Sequence
  heavy chain polypeptide with knob and VH and tag
EVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSHHHHHHHH

SEQ ID NO: 16 - protein, 461 aa, Artificial Sequence; identical to SEQ ID NO: 14
  anti-LeY heavy chain polypeptide with knob and VL anti-CD3

SEQ ID NO: 17 - protein, 240 aa, Artificial Sequence
  heavy chain polypeptide with hole and VH and tag
EVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSHHHHHHHH

SEQ ID NO: 18 - protein, 468 aa, Artificial Sequence; identical to SEQ ID NO: 12
  anti-LeY heavy chain polypeptide with hole and VH anti-CD3

SEQ ID NO: 19 - protein, 227 aa, Artificial Sequence
  heavy chain polypeptide with knob and VL and tag
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSHHHHHHHH

SEQ ID NO: 20 - protein, 594 aa, Artificial Sequence
  anti-LeY heavy chain polypeptide with knob and VH anti-CD3 and CH2
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGSGGEVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASGGGGSGGGGSGGSGGGEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 352 aa, Artificial Sequence
  heavy chain polypeptide with hole and VL and CH2 and tag
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKSSGGGGSGGGGSGGSGGGEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSHHHHHHHH

SEQ ID NO: 22 - protein, 587 aa, Artificial Sequence
  anti-LeY heavy chain polypeptide with hole and VL anti-CD3 and CH2
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGSGGDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKSSGGGGSGGGGSGGSGGGEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 370 aa, Artificial Sequence
  heavy chain polypeptide with knob and VH and CH2 domain and tag
QVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSASGGGGSGGGGSGGSGGGEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSHHHHHHHH

SEQ ID NO: 24 - protein, 587 aa, Artificial Sequence
  anti-LeY heavy chain polypeptide with knob and VL anti-CD3 and CH2
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGSGGDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKSSGGGGSGGGGSGGSGGGEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 370 aa, Artificial Sequence
  heavy chain polypeptide with hole and VH and CH2 and tag
QVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSASGGGGSGGGGSGGSGGGEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSHHHHHHHH

SEQ ID NO: 26 - protein, 594 aa, Artificial Sequence
  anti-LeY heavy chain polypeptide with hole and VH anti-CD3 and CH2
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGSGGEVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASGGGGSGGGGSGGSGGGEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 352 aa, Artificial Sequence
  heavy chain polypeptide with knob and VL and CH2 and tag
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKSSGGGGSGGGGSGGSGGGEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSHHHHHHHH

SEQ ID NO: 28 - protein, 450 aa, Artificial Sequence
  anti-biocytinamid heavy chain polypeptide with hole
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 448 aa, Artificial Sequence; identical to SEQ ID NO: 4
  anti-fluorescein heavy chain polypeptide with knob